(19) United States
(12) Patent Application Publication
(10) Pub. No.: US 2024/0315956 A1
(43) Pub. Date: Sep. 26, 2024

(54) METHOD OF PRODUCTION OF THE COMPOSITION OF CYCLOOXYGENASE-2 (COX-2) INHIBITORS

(71) Applicant: US NANO FOOD & DRUG INC, Wilmington, DE (US)

(72) Inventors: Hing Sang PUI, Sydney, NSW (AU); Yip Ching PUI, Sydney, NSW (AU); Yip Shu PUI, Sydney, NSW (AU)

(21) Appl. No.: 18/574,757

(22) PCT Filed: Jul. 8, 2022
(86) PCT No.: PCT/IB2022/056309

§ 371 Date: Dec. 28, 2023

(30) Foreign Application Priority Data
Jul. 9, 2021 (AU) 2021902100

(51) Int. Cl. A61K 9/46; A61K 9/00; A61K 31/194; A61K 31/4015; A61K 31/415; A61K 47/12; A61K 47/32; A61K 47/38

(52) U.S. Cl. CPC A61K 9/0007; A61K 9/0095; A61K 31/194; A61K 31/4015; A61K 31/415; A61K 47/12; A61K 47/32; A61K 47/38

(57) ABSTRACT

The disclosure is directed to the method of production of the composition of a cyclooxygenase-2 (COX-2) inhibitor comprising a water soluble excipient comprising HPMC, an organic acid and one or more other components. The composition may be in the form of pellets, a tablet, a capsule or granules with higher dissolution of the API or fast disintegration in the aqueous medium. In some embodiments, the composition further contains a carbonate salt to form a weakly effervescent disintegration formulation.

### FIELD OF THE INVENTION

The present invention broadly relates to pharmaceutical formulations and compositions, methods of producing and methods of use thereof. More particularly, the invention relates to producing pharmaceutical formulations and compositions comprising an active ingredient, and an organic acid, having improved dissolution properties.

### CROSS REFERENCE TO RELATED APPLICATION

This application claims priority to Australian Provisional Application No. 2021902100, which is herein incorporated by cross-reference in its entirety.

### BACKGROUND OF THE INVENTION

Any drug to be absorbed must be present in the form of a solution at the site of absorption. Various techniques are used for the enhancement of the solubility of poorly soluble drugs which include physical and chemical modifications of drug and other methods like particle size reduction, crystal engineering, salt formation, solid dispersion, use of surfactant, complexation, and so forth. The oral bioavailability depends on several factors including aqueous solubility, drug permeability, dissolution rate, first-pass metabolism, presystemic metabolism. The most frequent causes of low oral bioavailability are attributed to poor solubility and low permeability. The term “soluble” is sometimes used for materials that may form colloidal suspensions of very fine solid particles in a liquid.

It is estimated that about 50% of people have difficulty swallowing tablets and capsules, which significantly impacts drug treatment compliance. In other cases, many patients do not have water or an alternative fluid readily hand when needing to swallow a tablet or capsule. Orally disintegrating tablets (also referred to herein as oral disintegration tablets) (ODTs) and dispersible tablets may be administered to a patient by placing the tablet on the tongue or below the tongue to facilitate drug delivery without the need for drinking water or other fluids to assist swallowing.

Poorly water soluble drugs often require high doses in order to reach therapeutic plasma concentrations after oral administration.

These poorly water soluble drugs having slow drug absorption leads to inadequate and variable bioavailability and gastrointestinal mucosal toxicity. For orally administered drugs, solubility is the most important one rate limiting parameter to achieve their desired concentration in systemic circulation for pharmacological response.

The solubility of drug is often related to drug particle size; as a particle becomes smaller, the surface area to volume ratio increases. The larger surface area allows greater interaction with the solvent which causes an increase in solubility.

Celecoxib (CEL) and imrecoxib are selective cyclooxygenase-2 (COX-2) inhibitor therapeutically indicated for the treatment of rheumatoid arthritis, osteoarthritis, acute pain, and inflammation. Celecoxib is a white or almost white, crystalline or amorphous powder, hydrophobic (log P is 3.0) and practically insoluble in water. However, its poor solubility and dissolution rate significantly hinders its absorption in the intestine which in turn needs to be administered in large amounts of celecoxib but having low bioavailability, this leads to large amount of unabsorbed celecoxib in the alimentary system and causes many alimentary side effects such as peptic ulcer, gastric erosion, bleeding or perforation of stomach.

The strength of celecoxib on the market is 200 mg per capsule and the dosage is twice daily as needed. If the celecoxib is in tablet form then the release of drug or dissolution of the celecoxib is smaller than the capsules and cannot maintain an appropriate blood concentration. Therefore the celecoxib of prior art is a capsule on the market but the speed of production of the capsules is lower than the tablet and the cost of production of capsule is higher than the tablet. There is no tablet of celecoxib currently on the market.

Imrecoxib is similar to celecoxib (CEL) and is a selective cyclooxygenase-2 (COX-2) inhibitor therapeutically indicated for the treatment of rheumatoid arthritis, osteoarthritis, acute pain, and inflammation. Imrecoxib is a white or almost white, crystalline or amorphous powder, hydrophobic and practically insoluble in water. It will cause similar side effect as celecoxib.

Calcium carbonate is another poor solubility drug. It appears as white, odorless powder or colorless crystals. Calcium carbonate has a very low solubility in pure water (15 mg/L at 25° C.). Calcium carbonate is a calcium supplement for preventing and treating osteoporosis. Calcium carbonate tablets on the market include, for example, USP 1250 mg per tablet. Such tables are used for calcium deficiency. The dosage recommendations for adult is 0.5-4 g daily in 1-3 divided doses.

In the body, calcium is a mineral that makes up bones, as well as a salt that dissolves in the blood and regulates bodily function. The normal range for blood calcium level is 8.5 to 10.3 mg/dL. For a person of 70 kg body weight, the amount of blood is about 5-6 litres. The total amount of calcium ions in the blood is below 630 mg. For 4 gram of calcium carbonate, the amount of calcium ions is 1600 mg. Obviously, 90% of the calcium carbonate ingested is wasted and passes through and out of the large intestine, which may cause constipation.

If we can provide a new formulation of a water insoluble API with high dissolution rate, then the amount of water insoluble drug administered will be less, and its side effects may be reduced. The need for a high dissolution rate of celecoxib, imrecoxib and calcium carbonate is not met.

For water insoluble API and large dosage drug, such as 1500 mg calcium carbonate tablets and celecoxib 400 mg tablets, the amount of alimentary fluid needed for disintegration of the drug is very high, the time of disintegration is overly long and the window of absorption in the alimentary system is low. It is preferable to make the drug disintegrate faster by adding some expander such that the formulation is in the form of a dispersible tablet or an oral disintegration tablet which significantly shortens the drug disintegration time to about one to three minutes, increases the surface area of the granules and powder to interact with water to increase drug dissolution and absorption. Orally disintegrating tablets and dispersible tablets may be administered to a patient by placing the tablet on the tongue or below the tongue to facilitate drug delivery without the need for drinking water or other fluids to assist swallowing.

The term “orodispersible tablet” is used by the European Pharmacopoeia to refer to tablets that disperse inside the mouth readily and within 3 minutes before swallowing. The term “orally disintegrating tablets” or ODTs is used by the European Pharmacopoeia and United States Pharmacopoeia (USP) to refer to tablets that disintegrate inside the oral cavity within 1 minute. The testing equipment for the time of disintegration of these two kinds of tablets is the same.

ODTs are formulated with the aim of improving the disintegration of a pharmaceutical product to facilitate administration of a pharmaceutical substance via the oral cavity. In order to achieve rapid disintegration rates, suitable tablet formulations must provide high porosity, low density and a low hardness. This type of dosage form is often chosen when a patient has difficulty in swallowing, and is also suitable for use in geriatric and pediatric patients, or for those who suffer from conditions such as dysphagia, or patients who are bedridden and may not have ready access to water or an alternative liquid to facilitate swallowing.

Dosage forms that disintegrate in the presence of very small amounts of water may offer one or more advantages, including for example, good stability, accurate dosing, ease of manufacturing, small packaging size, and they are easily handled by patients. Orally disintegrating dosage forms, including weakly effervescent orally disintegrating tablet formulations, may also advantageously minimise or prevent the risk of obstruction of the gastrointestinal tract, which may be particularly beneficial for patients who do not have ready access to water or other fluids, and may allow for easy administration to pediatric patients, geriatric patients, patients suffering mental or cognitive illness, and psychiatric patients. Other advantages of orally disintegrating dosage forms include mitigation or prevention of the risk of asphyxia caused by tablets becoming lodged or stuck in the trachea, or choking if tablets become lodged in the oesophagus. For example, in the event of an orally dispersible tablet or ODT becoming lodged in the trachea or oesophagus of a patient, an advantage of the tablet according to the present invention is that it will disintegrate partially within 1 minute or completely within 3 minutes, enabling the patient's respiration to recover. The fast dissolution and fast absorption of a drug may also provide a rapid onset of action.

Dispersible tablets typically disintegrate in water within about 3 minutes and are generally administered in two ways: (1) dispersing the dosage form in a glass of aqueous liquid or solution (such as water, juice, or the like), and then the resultant suspension or solution is taken by mouth, or (2) the dispersing dosage form is placed in the oral cavity without the need for drinking water or other fluid.

Dispersible tablets may be made by well-known techniques, including compression of granules with a large amount of a physically disintegrating excipient (expander), such as cross linked povidone, or cross linked sodium carboxymethyl cellulose. Typically, the expander will be present in an amount which is more than 50% of the total weight of the tablet.

Various processes may be employed to manufacture ODTs such as lyophilization, molding, cotton candy process, spray drying, mass extrusion, compaction, and other well-known technologies. A disadvantage of the lyophilization process is that it requires expensive equipment and is complicated compared with the standard manufacturing process. A further drawback is that ODTs prepared by the lyophilization process also require special packaging material.

There is a particular need for orally disintegrating tablets or dispersible tablets for large tablet formulations, for example, to aid in administration and prevent or mitigate the risk of asphyxia. There is also a demand for ODTs that may disintegrate in very small volumes of water and rapidly disintegrate in the mouth for use in delivering medication to pediatric patients and the elderly.

Other advantages offered by orally disintegrating tablets is that they may solve or substantially mitigate the problem of drug treatment non-compliance, by enabling a drug to be taken without water. It is also possible that the bioavailability of the drug delivered as an orally dispersible tablet or ODT may be higher than that of alternative dosage forms, and side effects caused by the first pass metabolism may be reduced.

Rapid disintegrating tablets, also known as rapid dispersing preparations, have advantages over alternative formulations for improving the compliance of patients, such as effervescent tablets, suspensions, chewing gum and chewable tablets.

Orally disintegrating tablets may be particularly useful in one or more of the following exemplary scenarios:
1. First aid drugs that may be absorbed through oral mucosa, such as nitroglycerin and nifedipine.
2. Medication for patients with dysphagia (such as oesophageal cancer patients).
3. Antiemetic drugs, such as ondansetron, ramosetron, granisetron, metoclopramide, etc.
4. Patients who do have impaired cognitive function or who are non-compliant in taking medications, such as antidepressant and antipsychotic medications.
5. Medication for children, the elderly or bedridden patients who are not always readily able to access water or other fluids.
6. Emergency conditions such as epilepsy seizure, angina pectoris, myocardial infarction.

A challenge in developing rapid disintegrating tablets is the requirement for good physical properties and disintegrating properties. At present, disintegration tests for oral disintegrating tablets are not specified in the USP and European Pharmacopoeia and the results of tests of dispersible tablets may only approximate the actual disintegration time of ODTs in the mouth.

Methods for the production of fast disintegration tablets include freeze drying, spray drying, wet granulation, dry granulation and direct compression. Each of these general processes suffer from processing drawbacks. For example, ODTs made by lyophilization comply with the Chinese Pharmacopoeia (CP), however lyophilization machinery is expensive, and the production process is time consuming and complicated. ODTs produced by the freeze-drying method also have unqualified fragility (higher than 1%) and low tablet weight. In addition, a particular drawback is that the freeze drying method cannot produce an ODT having an active pharmaceutical ingredient (API) in an amount of more than 50 mg.

The evaluation method of oral disintegration tablets is specified in Chinese Pharmacopoeia (CP) and limits the moving up and down of the stainless tube containing the ODT of no more than 10 mm±1 mm. Many oral disintegration tablets on the market in the USA do not comply with the Chinese Pharmacopoeia, other than ODTs produced by lyophilization.

Examples of ODTs manufactured using ordinary excipients include APIs selected from acetaminophen, rofecoxib, tramadol, diphenhydramine, domperidone, zolmitriptan, cetirizine, zolpidem tartrate, and ibuprofen. Examples of ODTs known in the field that are made by the lyophilization method include APIs selected from ondansetron, ramosetron, granisetron, loratadine, piroxicam, benzoic acid, famotidine, olanzapine, risperidone, tiposaline, selegiline, clonazepam, and loperamide hydrochloride.

The typical composition of a lyophilized ODT includes the API, matrix and other excipients. After lyophilization, the preparation has a certain shape and strength. In order to make the lyophilized tablets loose and porous as required for an ODT, the key step is rapid freezing. In order to ensure the API is evenly distributed in the suspension prior to lyophilization, long-chain polymer substances, such as polypeptide (with gelatin or dehydrated gelatin), polysaccharide (dextran, mannitol, starch, etc.), gum (with Arabic gum, xanthan gum), fibrinogen, alginate, PVP, polyvinyl alcohol, etc., may be added. Other excipients may be added according to the specific composition desired, such as a wetting agent (ethanol), colorant (iron oxide), preservative, antioxidant and perfume.

The principle of preparing ODTs by the freeze drying method is that the API is made into a suspension, then quickly frozen into a solid, and then sublimated directly from the frozen state to remove water under vacuum. Accordingly, the API must be insoluble in water. Therefore, this method is unsuitable for water soluble APIs as after sublimation of the water, the solution will become an amorphous powder and no tablet may be formed. Results show that the freeze dried products are generally loose in structure, rich in small pores, and may quickly absorb water to disintegrate.

The US Federal Drug Administration (FDA) also found that although the disintegration time of these products ranged from a few seconds to more than a minute, the disintegration time of most products was about 30 seconds or less. This means that different production technologies, different size and weight of tablets, different volumes of disintegration liquid, and different mechanisms of disintegration result in different disintegration times. Other parameters that need to be considered in the development of suitable ODTs and production methods include the tablet size, tablet weight, solubility of the API and the influence of these factors on the development purpose of such products. In addition, the preferred weight of tablets recommended by the FDA is 500 mg or less.

A need remains for an ODT that may be manufactured on an industrial scale using a simple method with low cost and ordinary excipients, and which preferably complies with the Chinese Pharmacopoeia (CP).

For water soluble APIs, the faster the disintegration in the stomach, the more will be absorbed by the patient. But it is found this is not true for water insoluble APIs such as celecoxib, imrecoxib and calcium carbonate. Therefore, a new formulation with higher dissolution or dissociation is needed for the market to reduce the alimentary side effect.

Celecoxib is a weakly acidic compound which does not dissociate easily in the gastric juice as the HCl in the gastric juice will inhibit the dissociation of the weak acid in the aqueous medium due to common ion effect, which will reduce the absorption of celecoxib.

For the celecoxib capsules on the market, though the time to reach Cₘₐₓ is 3 hours, the Cₘₐₓ is very low at about 700 ng/mL. If a woman of 60 kg body weight has a blood volume of 5000 mL, then the total amount of celecoxib in the blood at the Cₘₐₓ is only 3.5 mg, which is only 1.75% of the drug ingested (200 mg capsule). Most of the celecoxib ingested is not absorbed, which acts as a toxic substance in the intestine, potentially causing side effects.

There is no micronized celecoxib tablet or capsule on the market. No products have a higher dissolution rate than the celecoxib of original producer. It is hoped that the new composition will have higher dissolution of celecoxib and therefore the composition may have less API per unit dosage such that the new composition comprises: 160 mg of celecoxib per composition, 150 mg of celecoxib per composition, 140 mg of celecoxib per composition, 130 mg of celecoxib per composition and each of these compositions is bioequivalent with the 200 mg celecoxib capsules of the original producer, such that the intestinal side effect of the invention is much lower than for the 200 mg celecoxib capsule of the original producer, while the efficacy remains substantially the same. The formulation of the composition may be in the form of granules, tablets or capsules.

### SUMMARY OF THE INVENTION

The present invention broadly relates to pharmaceutical formulations and methods of use. More particularly, the invention relates to pharmaceutical formulations comprising an active ingredient, and an organic acid, having improved dissolution properties.

Embodiments of the invention relate to a method of production of the composition of cyclooxygenase-2 (COX-2) inhibitors comprising celecoxib and imrecoxib, a water soluble excipient comprising HPMC and an organic acid such that the composition has high dissolution rate of COX-2 inhibitor or fast disintegration in an aqueous medium. In some situations, the composition further contains a carbonate salt to form the weakly effervescent disintegration formulation.

The composition of the selective cyclooxygenase-2 (COX-2) inhibitor comprises celecoxib or imrecoxib, an organic acid selected from the group including ascorbic acid, malic acid, maleic acid, succinic acid, tartaric acid and citric acid or a mixture thereof; the composition further contains cross linked povidone, cross linked sodium carboxymethyl cellulose, microcrystalline cellulose and lubricating agent of magnesium stearate, and mixtures thereof. The composition may be in the form of a tablet, granules, a capsule, a dispersible tablet or an oral disintegration tablets.

Another composition of the selective cyclooxygenase-2 (COX-2) inhibitor comprises celecoxib, hydroxypropyl methylcellulose (HPMC E5) and pellets which is made with a sugar or mannitol; wherein the formulation is in the form of a capsule or pellet.

Another composition of the selective cyclooxygenase-2 (COX-2) inhibitor comprises imrecoxib, HPMC E5 and pellets which is made with a sugar or mannitol; wherein the formulation is in the form of a capsule or pellets in a bag.

Another composition comprises celecoxib, an organic acid selected from the group consisting of malic acid, maleic acid, succinic acid, ascorbic acid, tartaric acid and citric acid or a mixture thereof and calcium carbonate; the composition further comprises cross linked povidone, cross linked sodium carboxymethyl cellulose, microcrystalline cellulose, and magnesium stearate and mixtures thereof. The composition may be in the form of a tablet, granules, a capsule, a dispersible tablet or oral disintegration tablet. This composition also increase both the dissolution of calcium ions and celecoxib in aqueous media. The combination of calcium carbonate with celecoxib and an organic acid decreases the amount of calcium carbonate needed.

Another composition of the invention contains imrecoxib and one organic acid selected from the group including malic acid, maleic acid, succinic acid, ascorbic acid, tartaric acid and citric acid or a mixture thereof; the composition further contains cross linked povidone, cross linked sodium carboxymethyl cellulose and lubricating agent of magnesium stearate, and mixtures thereof. The composition may be in the form of a tablet, granules or a capsule.

Another composition of the invention comprises imrecoxib, calcium carbonate, an organic acid selected from the group consisting of malic acid, maleic acid, succinic acid, ascorbic acid, tartaric acid and citric acid or a mixture thereof, cross linked povidone, cross linked sodium carboxymethyl cellulose, and lubricating agent of magnesium stearate and mixtures thereof. The composition may be in the form of a tablet, granules or a capsule.

Another composition of the invention comprises calcium carbonate and an organic acid selected from the group including malic acid, maleic acid, succinic acid, ascorbic acid, tartaric acid and citric acid or a mixture thereof; the composition further contains cross linked povidone, microcrystalline cellulose, lubricating agent of magnesium stearate and mixtures thereof. The range of dissolution of calcium ions of the composition of the invention comprising calcium carbonate is from 10% to 75% in 900 mL of neutral pure water under stirring at a speed of 75 RPM when measured at 30 min, and the pH of the resultant solution of disintegration is more than 4.9, such that the formulation may be in the form of a tablet, granules, a capsule, a weakly effervescent oral disintegration tablet or a dispersible tablet.

The dissociation rate of conventional tablet of calcium carbonate of 1500 mg on the market is 100% in 200 mL of gastric acid but is only about 1.3% in 900 mL of neutral pure water. In general, human males do not have excessive amounts of gastric fluid in the stomach, therefore the digestion of calcium carbonate in males is very limited.

The rate of dissociation of calcium ions of calcium carbonate tablet will be increased slightly if it contains a small amount of weak acid, such that the pH of the solution is more than 4.9 after a tablet of invention is disintegrated in 200 mL of neutral pure water, and the dissociation of calcium ions is in the range of 45% to 75% when the ratio of binary acid to calcium carbonate is from 0.35:1 to 1:1 by molar ratio. The dissociation of calcium ions is in the range of 24% to 75% when the ratio of binary acid to calcium carbonate is from 0.15:1 to 1:1 by molar ratio. The weak acid is selected from ascorbic acid, malic acid, maleic acid, succinic acid and tartaric acid, and the amount of weak acid is limited to a range such that the final pH of the solution of disintegration is above 4.9.

Another need remains for an calcium carbonate ODT, dispersible tablet, or tablet with a certain amount of organic acid together to form a high dissociation composition and a pH of more than 4.9 after disintegration in neutral pure water. Therefore the dosage of calcium carbonate may be smaller than 150 mg calcium carbonate each day, may be 100 mg calcium carbonate each day, may be 75 mg calcium carbonate each day or may be 40 mg calcium carbonate each day, but the calcium ions absorbed is much more than the conventional tablet of 1500 mg per tablet.

It was found the dissociation of calcium ions of calcium carbonate tablet on the market is only 1.4% in 900 mL of neutral pure water. We know the blood concentration of calcium ions of human beings is about 9.2 mg/dL or 92 mg per litre. A human being of 70 kg has about 5.5 litres of blood and the total calcium ions in the blood is about 500 mg.

In adults, one to two kilograms of calcium ions are stored in the body (the average being 1100 g). 99% of this store is in the bones which serve as a large reservoir. Only 1% is present in the plasma and about 0.1% in the extracellular fluid.

The dosage of calcium carbonate tablet on the market is 1500 mg. If the calcium is 100% absorbed, the amount of ion absorbed is 1200 mg, this will be 2 times the total amount of calcium ions in the blood of a human body. Obviously, over 90% of calcium is not absorbed and remains in the large intestine and causes constipation. The invention of a calcium carbonate tablet of a higher dissociation composition is needed; this special composition has a dissociation rate of 24% to 75%, which depends on the relative amount of acid to the amount of calcium carbonate contained, which will decompose to calcium ions and carbon dioxide. Therefore the dosage of calcium carbonate consumed each day may be reduced to 75 mg per day but provide enough calcium to the human without inducing the side effect of a large dosage of calcium carbonate, such as constipation, belching and difficulty in swallowing. The moderate higher dissociation of a calcium carbonate tablet of the invention may prevent the occurrence of burning of the mouth when the formulation is in the form of an oral disintegration tablet.

The composition of the selective cyclooxygenase-2 (COX-2) inhibitor comprises celecoxib, imrecoxib, an organic acid selected from the group consisting of ascorbic acid, malic acid, maleic acid, succinic acid, tartaric acid and citric acid, hydroxypropyl methylcellulose (HPMC E5); the composition further contains cross linked povidone, cross linked sodium carboxymethyl cellulose, microcrystalline cellulose, magnesium stearate and mixtures thereof. The composition may be in the form of a tablet, granules, or a capsule.

Another composition of the selective cyclooxygenase-2 (COX-2) inhibitor comprises celecoxib, HPMC E5 (hydroxypropyl methylcellulose) and pellets, which is made with sugar or mannitol. The composition is made by dissolving 1 part of celecoxib, 1 part to 2 parts of HPMC E5 in 10-15 parts of 95% ethanol; the alcoholic solution of celecoxib with HPMC E5 is sprayed into the fluid bed containing 1 part to 2 parts of sugar pellet. After many layers of coating of the sugar pellet with celecoxib with HPMC E5 ethanol solution, the pellet will become increasingly larger. The strength of celecoxib per capsules is from 65 mg to 160 mg, and has a dissolution rate of celecoxib of more than 60% in 1000 mL of neutral pure water containing 2.5 g of sodium dodecyl sulfate under 50 RPM at 180 minutes after dissolution test.

Another composition of the selective cyclooxygenase-2 (COX-2) inhibitor comprises imrecoxib, HPMC E5 (hydroxypropyl methylcellulose) and pellets, which is made with a sugar or mannitol. The composition is made by dissolving 1 part of imrecoxib, 1 part to 2 parts of HPMC E5 in 10-15 parts of 95% ethanol; the alcoholic solution of imrecoxib with HPMC E5 is sprayed into the fluid bed containing 1 part to 2 parts of the sugar pellet. After many layers of coating of the sugar pellet with imrecoxib with HPMC E5 ethanol solution, the pellet will become increasingly larger. The strength of imrecoxib per capsules is from 40 mg to 80 mg, and has a dissolution rate of imrecoxib of more than 60% in 1000 mL of neutral pure water containing 2.5 g of sodium dodecyl sulfate under 50 RPM at 180 minutes after dissolution test.

In one aspect, the invention relates to a weakly effervescent disintegration formulation comprising: a weak acid-base pair, wherein the weak acid is selected from a monosodium salt of a binary acid, monopotassium salt of a binary acid, monosodium salt of a ternary acid, a monopotassium salt of a ternary acid, ascorbic acid, and a small amount of binary acid, and the weak base is selected from calcium carbonate, magnesium carbonate, sodium bicarbonate, potassium bicarbonate and lithium bicarbonate; and an API; wherein the formulation disintegrates in neutral pure water to produce a pH greater than 4.9.

In an embodiment, the weakly effervescent disintegration formulation may be in the form of an immediate release tablet, an oral disintegration tablet, a dispersible tablet, a sublingual tablet or granules. The weight of the tablet determines the time of disintegration, which in turn determines whether it is an ODT or a dispersible tablet.

In an embodiment, the weak acid may be selected from ascorbic acid, monosodium malate, monosodium maleate, monosodium succinate, monosodium adipate, monosodium oxalate, monosodium citrate, monosodium tartrate, monosodium fumarate, monopotassium malate, monopotassium maleate, monopotassium succinate, monopotassium adipate, monopotassium oxalate, monopotassium citrate, monopotassium tartrate, monopotassium fumarate, a small amount of binary acid and mixtures thereof.

In an embodiment, the weakly effervescent disintegration formulation may comprise one weak acid only.

In an embodiment, the binary acid may be selected from tartaric acid, malic acid, maleic acid, adipic acid, fumaric acid, succinic acid and ascorbic acid.

In an embodiment, the weak acid may be a binary acid selected from malic acid, maleic acid, succinic acid, adipic acid, fumaric acid and tartaric acid, and the weak base is calcium carbonate, wherein the ratio of binary acid to calcium carbonate is less than 1 by molar ratio; and wherein the dissolution of calcium ions is in the range of 10% to 75% in 900 mL of neutral pure water under stirring at a speed of 75 RPM when measured at 30 min.

In an embodiment, the weak acid may be ascorbic acid and the weak base may be calcium carbonate only, wherein the ratio of ascorbic acid to calcium carbonate is from 1.0:1.0 to 4.0:1.0 by weight/weight ratio; and wherein the dissolution of calcium ions is in the range of 10% to 65% in 900 mL of neutral pure water under stirring at a speed of 75 RPM when measured at 30 min.

In an embodiment, the weakly effervescent disintegration formulation may further comprise one or more excipients selected from a filler, an expander, and a lubricant. The expander may be selected from cross linked povidone and cross linked carboxymethyl sodium cellulose. The lubricant may be selected from magnesium stearate, PEG 6000, colloidal silicon dioxide, and mixtures thereof.

In an embodiment, the API may be selected from vitamins, minerals, food nutrients, triptan drugs, 5-HT3 antagonist antiemetic drugs, dihydropyridine calcium channel blockers, antihistamines, sartans, alpha-glucosidase inhibitors, antifibrinolytic drugs, serotonin reuptake inhibitors, non-classical antidepressants, selective COX-2 inhibitors, NSAIDs, hormone contraceptives and anti-epileptics.

In preferred embodiments, the weak base is calcium carbonate.

In an embodiment, the weakly effervescent disintegration formulation comprises ascorbic acid, malic acid and calcium carbonate.

Another composition comprises calcium carbonate, glucosamine hydrochloride, cross linked povidone, cross linked carboxymethyl sodium cellulose, microcrystalline cellulose, magnesium stearate and mixtures thereof, the dissolution of calcium ions of the composition is from 37% to 55% and the final pH of disintegration is from 6.6 to 7.0.

Another weakly effervescent disintegration formulation of the invention comprises sodium bicarbonate, glucosamines sulfate, cross linked povidone, magnesium stearate, PVPK30, wherein the formulation is in the form of granules or a dispersible tablet, and has a pH in the range of 6.5 to 7.5 in 200 mL of neutral water.

Another weakly effervescent disintegration formulation of the invention comprises 500 mg of glucosamine hydrochloride, 200 mg of sodium bicarbonate, 200 mg of cross linked povidone, magnesium stearate, wherein the formulation is in the form of granules or a dispersible tablet and has a pH in the range of 6.5 to 7.5 in 200 mL of neutral water.

The composition of celecoxib contains celecoxib and one organic acid selected from the group consisting of ascorbic acid, malic acid, maleic acid, succinic acid, tartaric acid and citric acid or a mixture thereof, an excipient selected from cross linked carboxymethyl sodium cellulose, cross linked povidone, microcrystalline cellulose, magnesium stearate and mixtures thereof, wherein the formulation is in the form of granules, a capsule, a tablet or a dispersible tablet.

Another composition of the selective cyclooxygenase-2 (COX-2) inhibitor comprises celecoxib, HPMC E5 and pellets which is made with a sugar or mannitol. The composition is made by dissolving 1 part of celecoxib and 1 part to 2 parts of HPMC E5 in 10-15 parts of dehydrated ethanol; the alcoholic solution of celecoxib with HPMC E5 is sprayed into the fluid bed containing 1 part to 2 parts of sugar pellet. The coating of the pellet with celecoxib and HPMC E5 increases the diameter of the pellet until a suitable size is reached. The dissolution of the composition of the invention of 160 mg celecoxib pellet containing an HPMC E5 has a dissolution rate of more than 60% of celecoxib in 1000 mL of 0.25% sodium dodecyl sulfate of pH 7 sodium phosphate buffer solution under 50 RPM at 180 minutes after dissolution test; wherein the formulation is in the form of a capsule or pellet.

Another composition of the selective cyclooxygenase-2 (COX-2) inhibitor comprises imrecoxib, HPMC E5 and pellets which is made with a sugar or mannitol. The composition is made by dissolving 1 part of imrecoxib and 1 part to 2 parts of HPMC E5 in 10-15 parts of dehydrated ethanol; the alcoholic solution of imrecoxib with HPMC E5 is sprayed into the fluid bed containing 1 part to 2 parts of the sugar pellet. The dissolution of the composition of the invention of 80 mg of imrecoxib pellet containing an HPMC E5 has a dissolution rate of more than 60% of imrecoxib in 1000 mL of 0.25% sodium dodecyl sulfate of pH 7 sodium phosphate buffer solution under 50 RPM at 180 minutes after dissolution test; wherein the formulation is in the form of a capsule or pellet.

Another composition of imrecoxib comprises one organic acid selected from the group consisting of ascorbic acid, malic acid, maleic acid, succinic acid, tartaric acid and citric acid or a mixture thereof, an excipient selected from cross linked carboxymethyl sodium cellulose, cross linked povidone, microcrystalline cellulose, magnesium stearate, and mixtures thereof, wherein the formulation is in the form of granules, a capsule, a tablet or a dispersible tablet.

Another composition of the invention contains celecoxib, ascorbic acid, cross linked povidone, cross linked carboxymethyl sodium cellulose, microcrystalline cellulose, magnesium stearate, and mixtures thereof, wherein the formulation is in the form of granules, a capsule, a tablet or a dispersible tablet; wherein the ratio of ascorbic acid to celecoxib is from 1:1 to 6:1 by weight.

Another composition of the invention contains celecoxib, malic acid, cross linked povidone, cross linked carboxymethyl sodium cellulose, microcrystalline cellulose, magnesium stearate, and mixtures thereof, wherein the formulation is in the form of granules, a capsule, a tablet or a dispersible tablet; wherein the ratio of malic acid to celecoxib is from 1:1 to 6:1 by weight.

Another composition of the invention contains celecoxib, maleic acid, cross linked povidone, cross linked carboxymethyl sodium cellulose, microcrystalline cellulose, magnesium stearate and mixtures thereof, wherein the formulation is in the form of granules, a capsule, a tablet or a dispersible tablet; wherein the ratio of maleic acid to celecoxib is from 1:1 to 6:1 by weight.

Another composition of the invention contains celecoxib, succinic acid, cross linked povidone, cross linked carboxymethyl sodium cellulose, magnesium stearate and mixtures thereof, wherein the formulation is in the form of granules, a capsule, a tablet or a dispersible tablet; wherein the ratio of succinic acid to celecoxib is from 1:1 to 6:1 by weight.

Another composition of the invention contains celecoxib, tartaric acid, cross linked povidone, cross linked carboxymethyl sodium cellulose, magnesium stearate and mixtures thereof, wherein the formulation is in the form of granules, a capsule, a tablet or a dispersible tablet; wherein the ratio of tartaric acid to celecoxib is from 1:1 to 6:1 by weight.

Another composition of the invention contains celecoxib, citric acid, cross linked povidone, cross linked carboxymethyl sodium cellulose, magnesium stearate and mixtures thereof, wherein the formulation is in the form of granules, a capsule, a tablet or a dispersible tablet; wherein the ratio of citric acid to celecoxib is from 1:1 to 6:1 by weight.

The composition of the invention of 160 mg celecoxib tablet containing an organic acid has a dissolution of about 125 mg celecoxib in 1000 mL of 0.25% sodium dodecyl sulfate of pH 7 sodium phosphate buffer solution under 50 RPM at 120 minutes after dissolution test, which is almost the same as the amount of dissolution of a CELEBREX (celecoxib) capsule of 200 mg strength in the same dissociation environment and conditions. This means 1 tablet of the invention of 160 mg celecoxib is bioequivalent with a 200 mg CELEBREX capsule of the original producer in vitro.

The celecoxib or imrecoxib composition of the invention is produced by alcoholic solution as follows:
1. One part of celecoxib powder is placed in the tank with 12-20 parts of ethanol or benzyl alcohol.
2. One part to six parts of an organic acid selecting from malic acid, ascorbic acid, maleic acid, succinic acid, tartaric acid and citric acid or a mixture thereof is dissolved in the tank containing alcohol and celecoxib.
3. Some of the excipients including about 1 part of cross linked carboxymethyl sodium cellulose (croscarmellose sodium), 1 part of cross povidone, 0.1 part of sodium lauryl sulfate and microcrystalline cellulose is placed in a fluid bed, then the alcoholic solution of the celecoxib and organic acid is sprayed into the fluid bed. Then an aqueous solution of povidone acting as the binder is spayed into the fluid bed. The granules obtained are mixed with magnesium stearate and are then compressed into a tablet, or filled into a capsule or a granule bag.

The ratio of organic acid to COX-2 inhibitor including celecoxib or imrecoxib is from 1:1 to 6:1 by weight. The ratio of organic acid to celecoxib or imrecoxib is 1:1 by weight in one formula. The ratio of organic acid to celecoxib or imrecoxib is 2:1 by weight in another formula. The ratio of organic acid to celecoxib or imrecoxib is 3:1 by weight in another formula. The ratio of organic acid to celecoxib or imrecoxib is 4:1 by weight in another formula. The ratio of organic acid to celecoxib or imrecoxib is 5:1 by weight in another formula. The ratio of organic acid to celecoxib or imrecoxib is 6:1 by weight in another formula. The organic acid is selected from the group consisting of ascorbic acid, malic acid, maleic acid, succinic acid, tartaric acid and citric acid or a mixture thereof.

After the composition of the invention of celecoxib is disintegrated in aqueous solution, the dissolution of celecoxib tablet is much higher than the dissolution of celecoxib capsules of the same strength on the market. This will increase its absorption in the alimentary system and reduce its side effects compared with the celecoxib capsules of the same strength on the market.

Celecoxib may be used to relieve arthralgia. Many cases of arthralgia in patients is due to calcium deficiency. A combination formulation of celecoxib and calcium carbonate may reduce the amount of celecoxib needed to relieve the joint pain, but the amount of calcium carbonate consumed is very high and may be more than 1500 mg twice daily because the dissociation of calcium carbonate is low and is as little as about 1.4% in neutral pure water.

The composition containing celecoxib, calcium carbonate and one organic acid has a better therapeutic effect than a combination of celecoxib and calcium carbonate, because the organic acid may decompose the calcium carbonate in water to CO₂ and calcium ions which is easily absorbed than by using calcium carbonate tablets only. The organic acid is selected from the group including ascorbic acid, malic acid, maleic acid, succinic acid, tartaric acid and citric acid or a mixture thereof.

In the embodiment of the composition of celecoxib with calcium carbonate and an organic acid, the acid of the composition may increase the dissociation of celecoxib and its absorption. This may reduce the amount of celecoxib needed to be consumed to relieve the joint pain and thus reduce the alimentary side effect compared with the celecoxib capsules on the market. After the composition of the invention of celecoxib, calcium carbonate and an organic acid dissolves in the alimentary system, the acid will react with the calcium carbonate to relieve the calcium ions and speed up the disintegration of the tablet; this leads to higher dissociation of calcium ions and fast disintegration of the formulation, such formulation will:
1. Reduce the amount of celecoxib to be consumed and reduce the alimentary side effect caused by celecoxib,
2. Reduce the amount of calcium carbonate needed to be consumed to treat calcium deficiency, the supplement of calcium ions to treat calcium deficiency further reduce the amount of celecoxib needed to treat the joint pain and further reduce the side effect of celecoxib.
3. The higher dissolution of calcium carbonate and celecoxib will reduce both the strength of celecoxib and calcium carbonate needed to treat the joint pain.

The composition of the invention comprising celecoxib contains 65-200 mg celecoxib, 65-960 mg organic acid selected from the group consisting of ascorbic acid, malic acid, maleic acid, succinic acid, tartaric acid and citric acid or a mixture thereof, 50-160 mg cross povidone, 50-160 mg cross linked sodium carboxymethyl cellulose, povidone or polyvinylpyrrolidone (PVP), a small amount of magnesium stearate per composition such that the composition is in the form of granules, a capsule, a tablet or a dispersible tablet.

The composition of the invention of celecoxib contains 65-200 mg celecoxib, 65-960 mg of ascorbic acid, 50-160 mg cross linked povidone, 50-160 mg cross linked sodium carboxymethyl cellulose, a small amount of magnesium stearate and PVPK30 per composition such that the formulation is in the form of granules, a capsule, a tablet or a dispersible tablet.

The composition of the invention comprising celecoxib contains 65-200 mg celecoxib, 65-960 mg malic acid, 50-160 mg cross povidone, 50-160 mg cross linked sodium carboxymethyl cellulose, a small amount of magnesium stearate and PVPK30 per formulation such that the formulation is in the form of granules, a capsule, a tablet or a dispersible tablet.

The composition of the invention comprising celecoxib contains 65-200 mg celecoxib, 65-960 mg maleic acid, 50-160 mg cross povidone, 50-160 mg cross linked sodium carboxymethyl cellulose, a small amount of magnesium stearate and PVPK30 per formulation such that the formulation is in the form of granules, a capsule, a tablet or a dispersible tablet.

The composition of the invention comprising celecoxib contains 65-200 mg celecoxib, 65-960 mg succinic acid, 50-160 mg cross povidone, 50-160 mg cross linked sodium carboxymethyl cellulose, a small amount of magnesium stearate and PVPK30 per formulation such that the formulation is in the form of granules, a capsule, a tablet or a dispersible tablet.

The composition of the invention comprising celecoxib contains 65-200 mg celecoxib, 65-960 mg tartaric acid, 50-160 mg cross povidone, 50-160 mg cross linked sodium carboxymethyl cellulose, a small amount of magnesium stearate and PVPK30 per formulation such that the formulation is in the form of granules, a capsule, a tablet or a dispersible tablet.

The composition of the invention comprising celecoxib contains 65-200 mg celecoxib, 65-960 mg citric acid, 50-160 mg cross povidone, 50-160 mg cross linked sodium carboxymethyl cellulose, a small amount of magnesium stearate and PVPK30 per formulation, such that the formulation is in the form of granules, a capsule, a tablet or a dispersible tablet.

The composition of the invention comprising calcium carbonate contains both calcium carbonate and an organic acid; the ratio of binary acid to calcium carbonate is from 0.15:1 mole to 1:1 mole and the rate of dissolution of calcium ions is from about 20% to 75% under 75 RPM and at 30 minutes after the starting of the dissolution test in 900 mL of neutral pure water; wherein the composition disintegrates in 900 mL of neutral pure water to produce a pH of greater than 4.9, such that the composition is in the form of a tablet, a capsule, granules, an oral disintegration tablet or a dispersible tablet.

A composition of the invention contains 25 mg to 250 mg (0.25-2.5 mmol) of calcium carbonate and 0.0375 mmol to 2.5 mmol of binary acid, and the rate of dissolution of calcium ions is about 20% to 75% under 75 RPM and at 30 minutes after dissolution test in 900 mL of neutral pure water; wherein the composition disintegrates in neutral pure water to produce a pH of greater than 4.9, such that the composition is in the form of a tablet, a capsule, granules, an oral disintegration tablet or a dispersible tablet.

In an embodiment, the weakly effervescent disintegration formulation of the composition of the invention may comprise: calcium carbonate, a small amount of a binary weak acid selected from malic acid, maleic acid, succinic acid, adipic acid, fumaric acid and tartaric acid, an excipient selected from cross linked povidone and cross linked carboxymethyl sodium cellulose, microcrystalline cellulose, magnesium stearate, and mixtures thereof, wherein the formulation is in the form of an oral disintegration tablet, or a dispersible tablet complying with USP in the time of disintegration, or granules; and wherein the formulation has a dissolution of calcium of more than 20% in 900 mL of neutral pure water under stirring at a speed of 75 RPM when measured at 30 min. The composition further has a pH of more than 4.9 and a dissolution of calcium ions of from 20% to 75% in 900 mL of neutral pure water.

In an embodiment, the weakly effervescent disintegration formulation may comprise calcium carbonate as the sole weak base, and ascorbic acid as the weak acid. The weight ratio of ascorbic acid to calcium carbonate may be from 1.0:1.0 to 3.0:1.0. The formulation may further comprise one or more excipients selected from cross linked povidone, cross linked carboxymethyl cellulose sodium, microcrystalline cellulose, magnesium stearate and mixtures thereof. The formulation may have a dissolution of calcium of about 20% to 65% in 900 mL of neutral pure water under stirring at a speed of 75 RPM when measured at 30 min.

The weakly effervescent disintegration formulation may produce a pH of more than 4.9 after disintegration in 900 mL of neutral pure water and has a dissolution of calcium of from about 20% to 75% in 900 mL of neutral pure water containing a binary acid selecting from malic acid, maleic acid, succinic acid and tartaric acid.

Another embodiment of the composition of the invention contains calcium carbonate and malic acid, the mole ratio of malic acid to calcium carbonate is from 0.15:1 to 1:1 and the rate of dissolution of calcium ions is about 20% to 75% under 75 RPM and at 30 minutes after dissolution test in 900 mL of neutral pure water to produce a pH of greater than 4.9, such that the formulation is in the form of a tablet, a capsule, granules, an oral disintegration tablet or a dispersible tablet.

Another embodiment of the composition of the invention contains calcium carbonate and maleic acid, the mole ratio of maleic acid to calcium carbonate is from 0.15:1 to 1:1 and the rate of dissolution of calcium ions is about 20% to 75% under 75 RPM and at 30 minutes after dissolution test in 900 mL of neutral pure water to produce a pH of greater than 4.9, such that the formulation is in the form of a tablet, a capsule, granules, an oral disintegration tablet or a dispersible tablet.

Another embodiment of the composition of the invention contains calcium carbonate and succinic acid, the mole ratio of succinic acid to calcium carbonate is from 0.15:1 mole to 1:1 mole and the rate of dissolution of calcium ions is about 20% to 75% under 75 RPM and at 30 minutes after dissolution test in 900 mL of neutral pure water to produce a pH of greater than 4.9, such that the composition is in the form of a tablet, a capsule, granules, an oral disintegration tablet or a dispersible tablet.

Another embodiment of the composition of the invention contains calcium carbonate and tartaric acid, the mole ratio of tartaric acid to calcium carbonate is from 0.15:1 mole to 1:1 mole and the rate of dissolution of calcium ions is about 20% to 75% under 75 RPM and at 30 minutes after dissolution test in 900 mL of neutral pure water to produce a pH of greater than 4.9, such that the composition is in the form of a tablet, a capsule, granules, an oral disintegration tablet or a dispersible tablet.

Another embodiment of the composition of the invention contains calcium carbonate and citric acid, the mole ratio of citric acid to calcium carbonate is from 0.25:1 to 0.8:1 and the rate of dissolution of calcium ions is about 45% to 75% under 75 RPM and at 30 minutes after dissociation test in 900 mL of neutral pure water to produce a pH of greater than 4.9, such that the composition is in the form of a tablet, a capsule, granules, an oral disintegration tablet or a dispersible tablet.

Another embodiment of the composition of the invention contains 25 mg to 250 mg calcium carbonate and 25 mg to 750 mg ascorbic acid and the rate of dissolution of calcium ions is about 20% to 65% under 75 RPM and at 30 minutes after dissolution test in 900 mL of neutral pure water to produce a pH of greater than 4.9, such that the composition is in the form of a tablet, a capsule, granules, an oral disintegration tablet or a dispersible tablet.

Another embodiment of the composition of the invention comprises celecoxib, malic acid, cross linked povidone, cross linked sodium carboxymethyl cellulose and magnesium stearate, such that the composition is in the form of a tablet, a capsule, or granules; wherein the weight ratio of malic acid to celecoxib is from 1:1 to 6:1 by weight.

Another embodiment of the composition of the invention comprises celecoxib, malic acid, calcium carbonate, cross linked povidone, cross linked sodium carboxymethyl cellulose and magnesium stearate, such that the formulation is in the form of a tablet, or granules; wherein the weight ratio of malic acid to celecoxib is from 1:1 to 6:1 by weight.

Another embodiment of the composition of the invention comprises celecoxib, maleic acid, cross linked povidone, cross linked sodium carboxymethyl cellulose and magnesium stearate, such that the composition is in the form of a tablet, a capsule, or granules; wherein the weight ratio of maleic acid to celecoxib is from 1:1 to 6:1 by weight.

Another embodiment of the composition of the invention comprises celecoxib, maleic acid, calcium carbonate, cross linked povidone, cross linked sodium carboxymethyl cellulose, magnesium stearate, such that the composition is in the form of a tablet, granules, or a capsule; wherein the weight ratio of maleic acid to celecoxib is from 1:1 to 6:1 by weight.

Another embodiment of the composition of the invention comprises celecoxib, succinic acid, cross linked povidone, cross linked sodium carboxymethyl cellulose and magnesium stearate, such that the composition is in the form of a tablet, a capsule, or granules; wherein the weight ratio of succinic acid to celecoxib is from 1:1 to 6:1 by weight.

Another embodiment of the composition of the invention comprises celecoxib, succinic acid, calcium carbonate, cross linked povidone, cross linked sodium carboxymethyl cellulose and magnesium stearate, such that the formulation is in the form of a tablet, or granules; wherein the weight ratio of succinic acid to celecoxib is from 1:1 to 6:1 by weight.

Another embodiment of the composition of the invention comprises celecoxib, ascorbic acid, cross linked povidone, cross linked sodium carboxymethyl cellulose and magnesium stearate, such that the composition is in the form of a tablet, a capsule, or granules; wherein the weight ratio of ascorbic acid to celecoxib is from 1:1 to 6:1 by weight.

Another embodiment of the composition of the invention comprises celecoxib, ascorbic acid, calcium carbonate, cross linked povidone, cross linked sodium carboxymethyl cellulose and magnesium stearate, such that the composition is in the form of a tablet, or granules; wherein the weight ratio of ascorbic acid to celecoxib is from 1:1 to 6:1 by weight.

Another embodiment of the composition of the invention comprises celecoxib, tartaric acid, cross linked povidone, cross linked sodium carboxymethyl cellulose and magnesium stearate, such that the composition is in the form of a tablet, a capsule, or granules; wherein the ratio of tartaric acid to celecoxib is from 1:1 to 6:1 by weight.

Another embodiment of the composition of the invention comprises celecoxib, tartaric acid, calcium carbonate, cross linked povidone, cross linked sodium carboxymethyl cellulose and magnesium stearate, such that the formulation is in the form of a capsule, a tablet, or granules; wherein the ratio of tartaric acid to celecoxib is from 1:1 to 6:1 by weight.

Another embodiment of the composition of the invention comprises celecoxib, citric acid, cross linked povidone, cross linked sodium carboxymethyl cellulose and magnesium stearate, such that the composition is in the form of a tablet, a capsule, or granules; wherein the ratio of citric acid to celecoxib is from 1:1 to 6:1 by weight.

Another embodiment of the composition of the invention comprises celecoxib, citric acid, calcium carbonate, cross linked povidone, cross linked sodium carboxymethyl cellulose and magnesium stearate, such that the composition is in the form of a tablet, or granules; wherein the ratio of citric acid to celecoxib is from 1:1 to 6:1 by weight.

Another embodiment of the composition of the invention comprises imrecoxib, malic acid, cross linked povidone, cross linked sodium carboxymethyl cellulose and magnesium stearate, such that the composition is in the form of a tablet, a capsule, or granules; wherein the weight ratio of malic acid to imrecoxib is from 1:1 to 6:1 by weight.

Another embodiment of the composition of the invention comprises imrecoxib, maleic acid, cross linked povidone, cross linked sodium carboxymethyl cellulose and magnesium stearate, such that the composition is in the form of a tablet, a capsule, or granules; wherein the weight ratio of maleic acid to imrecoxib is from 1:1 to 6:1 by weight.

Another embodiment of the composition of the invention comprises imrecoxib, succinic acid, cross linked povidone, cross linked sodium carboxymethyl cellulose and magnesium stearate, such that the composition is in the form of a tablet, a capsule, or granules; wherein the weight ratio of succinic acid to imrecoxib is from 1:1 to 6:1 by weight.

Another embodiment of the composition of the invention comprises imrecoxib, ascorbic acid, cross linked povidone, cross linked sodium carboxymethyl cellulose and magnesium stearate, such that the composition is in the form of a tablet, a capsule, or granules; wherein the weight ratio of ascorbic acid to imrecoxib is from 1:1 to 6:1 by weight.

Another embodiment of the composition of the invention comprises imrecoxib, tartaric acid, cross linked povidone, cross linked sodium carboxymethyl cellulose and magnesium stearate, such that the composition is in the form of a tablet, a capsule, or granules; wherein the ratio of tartaric acid to imrecoxib is from 1:1 to 6:1 by weight.

Another embodiment of the composition of the invention comprises imrecoxib, citric acid, cross linked povidone, cross linked sodium carboxymethyl cellulose and magnesium stearate, such that the composition is in the form of a tablet, a capsule, or granules; wherein the ratio of citric acid to imrecoxib is from 1:1 to 6:1 by weight.

In an embodiment of the weakly effervescent disintegration formulation, the weak base may be calcium carbonate and the weight of the calcium carbonate per tablet may be less than 150 mg. The binary acid may be selected from tartaric acid, malic acid, maleic acid, adipic acid, fumaric acid and succinic acid and the ratio of binary acid to calcium carbonate may be in the range of 0.35:1.0 to 1.0:1.0 molar ratio.

Advantageously, in an embodiment of the weakly effervescent disintegration formulation the dissolution of calcium ions may be in the range of about 10% to about 75% in 900 mL of neutral pure water under stirring at a speed of 75 RPM when measured at 30 min. In alternative embodiments, the weakly effervescent disintegration formulation may have a dissolution of calcium ions of from about 10% to 70%, or about 10% to 65%, or about 10% to 60%, or about 15% to 75%, or about 15% to 70%, or about 15% to 65%, or about 15% to 60%, or about 20% to 75%, or about 20% to 70%, or about 20% to 65%, or about 20% to 60%, or about 30% to 65%, or about 40% to 60% in 900 mL of neutral pure water under stirring at a speed of 75 RPM when measured at 30 min.

In an embodiment of the composition of the invention of calcium carbonate, the weight of the calcium carbonate per tablet may be less than or equal to 120 mg, 100 mg, 90 mg, 75 mg, 60 mg and 40 mg. The weak acid may be selected from ascorbic acid, citric acid, malic acid, tartaric acid, maleic acid and succinic acid. The amount of the acid in the formulation is limited by the final pH of calcium carbonate disintegration in the solution and produces pH of more than 4.9 after disintegration in 900 mL of neutral pure water, and has a dissolution of calcium ions of from about 20% to 75% in 900 mL of neutral pure water. The dosage of the composition of the invention of calcium carbonate is 120 mg, 100 mg, 90 mg, 75 mg, 60 mg and 40 mg once daily for adult and 60 mg, 40 mg or 20 mg once daily for children such that the formulation is in the form of granules, a capsule, a tablet, an oral disintegration tablet or a dispersible tablet.

In an embodiment, the weakly effervescent disintegration formulation may comprise calcium carbonate; ascorbic acid; and an API selected from food nutrients selected from vitamin A, vitamin B1, vitamin B2, vitamin B6, vitamin B12, vitamin D or D3, vitamin E, vitamin K, nicotinic acid, folic acid, pantothenic acid, choline, biotin, glucosamine salt and mixtures thereof. The formulation may be in the form of a dispersible tablet having a dissolution of calcium from 20% to 65% in 900 mL of neutral pure water and under stirring at a speed of 75 RPM when measured at 30 min.

In a preferred embodiment, the calcium carbonate may be the sole source of calcium in the tablet.

In a preferred embodiment, the amount of calcium carbonate contained per tablet is less than 75 mg per tablet per day consumed, or less than 50 mg per tablet per day consumed or 25 mg per tablet per day consumed. In an embodiment, the rate of dissolution of the calcium per tablets is from 20% to 76% when the pH of the resultant solution of the disintegration is greater than 5. In an embodiment, the weakly effervescent disintegration formulation comprises ascorbic acid, calcium carbonate, vitamin D3 and a glucosamine salt selected from glucosamine HCl and glucosamine sulfate; wherein the formulation further comprises one or more excipients selected from cross linked povidone, cross linked carboxymethyl sodium cellulose, microcrystalline cellulose and magnesium stearate, wherein the formulation is in the form of a dispersible tablet and produces a pH of more than 4.9 after disintegration in neutral pure water, and a dissolution of calcium of more than 10%, preferably more than 20% and is in the range of 20-80% in 900 mL of neutral pure water under stirring at a speed of 75 RPM when measured at 30 min.

In an embodiment, the weakly effervescent disintegration formulation comprises calcium carbonate, ascorbic acid, and a food nutrient selected from vitamin A, vitamin B1, vitamin B2, vitamin B6, vitamin B12, vitamin D3, vitamin E, vitamin K, nicotinic acid, folic acid, pantothenic acid, choline, biotin, iron, copper, zinc, magnesium, potassium, chloride, iodide, manganese, salts thereof, and mixtures thereof, wherein the formulation further comprises one or more excipients selected from cross linked povidone, cross linked carboxymethyl sodium cellulose, microcrystalline cellulose, magnesium stearate, and wherein the formulation is in the form of an oral disintegration tablet or a dispersible tablet which produces a pH of more than 4.9 after disintegration in neutral pure water.

In an embodiment, the weakly effervescent disintegration formulation comprises an API which is an alpha-glucosidase inhibitor selected from miglibose (miglitol), acarbose and voglibose, calcium carbonate, a citric acid or a binary acid selected from malic acid, maleic acid, adipic acid, fumaric acid, tartaric acid, succinic acid, ascorbic acid, and mixtures thereof. The formulation may additionally comprise one or more excipients selected from cross linked povidone, cross linked carboxymethyl sodium cellulose, microcrystalline cellulose, and magnesium stearate.

In an embodiment, the weakly effervescent disintegration formulation comprises acarbose, calcium carbonate, malic acid, cross linked povidone, microcrystalline cellulose, menthol, aspartame, and magnesium stearate, wherein the formulation produces a pH of more than 4.9 after disintegration in 900 mL of neutral pure water.

Another composition of the invention comprises acarbose, calcium carbonate, citric acid, cross linked povidone, microcrystalline cellulose, magnesium stearate, wherein the formulation produces a pH of more than 4.9 after disintegration in 900 mL of neutral water and the formulation is in the form of an oral disintegration tablet.

Another composition of the invention comprises acarbose, calcium carbonate, an acid selected from a group consisting of citric acid, malic acid, maleic acid, tartaric acid, succinic acid, ascorbic acid, and mixtures thereof. The formulation may additionally comprise one or more excipients selected from cross linked povidone, cross linked carboxymethyl sodium cellulose, microcrystalline cellulose, and magnesium stearate, wherein the formulation produces a pH of more than 4.9 after disintegration in 900 mL of neutral water and the formulation is in the form of an oral disintegration tablet.

Another composition of the invention comprises miglitol, calcium carbonate, an organic acid selected from a group consisting of a citric acid, malic acid, maleic acid, tartaric acid, succinic acid, ascorbic acid, cross linked povidone, microcrystalline cellulose, and magnesium stearate, wherein the formulation produces a pH of more than 4.9 after disintegration in 900 mL of neutral water and the formulation is in the form of an oral disintegration tablet.

In an embodiment, the weakly effervescent disintegration formulation comprises calcium carbonate; an acid selected from malic acid, maleic acid, succinic acid, adipic acid, fumaric acid, tartaric acid, ascorbic acid, and mixtures thereof; cross linked povidone, cross linked carboxymethyl sodium cellulose; and a hormonal contraceptive selected from desogestrel, norgestrel, levonorgestrel, megestrol, norethisterone, norgestimate, norethisterone oxime, ethinylestradiol, desethinylestradiol, quinestrol, gestestrone, megestrol acetate, progesterone, and mifepristone, wherein the formulation is in the form of an oral disintegration tablet or a dispersible tablet which produces a pH of more than 4.9 after disintegration in neutral pure water.

In an embodiment, the weakly effervescent disintegration formulation comprises calcium carbonate, malic acid, cross linked povidone, microcrystalline cellulose, menthol, aspartame, magnesium stearate, and levonorgestrel, wherein the formulation is in the form of an oral disintegration tablet which produces a pH of more than 4.9 after disintegration in neutral pure water.

In an embodiment, the weakly effervescent disintegration formulation comprises calcium carbonate, malic acid, cross linked povidone, microcrystalline cellulose, menthol, aspartame, magnesium stearate, and mifepristone, wherein the formulation is in the form of an oral disintegration tablet or a dispersible tablet which produces a pH of more than 4.9 after disintegration in neutral pure water.

In an embodiment, the weakly effervescent disintegration formulation comprises calcium carbonate, malic acid, cross linked povidone, microcrystalline cellulose, menthol, aspartame, magnesium stearate, and quinestrol, wherein the formulation is in the form of an oral disintegration tablet which has a pH of more than 4.9 after disintegration in neutral pure water.

A composition of the invention comprises progesterone, calcium carbonate, an acid selected from a group consisting of malic acid, maleic acid, succinic acid, tartaric acid, ascorbic acid, citric acid, and cross linked povidone, wherein the formulation is in the form of an oral disintegration tablet or a dispersible tablet which produces a pH of more than 4.9 after disintegration in 900 mL of neutral pure water.

In an embodiment, the weakly effervescent disintegration formulation comprises calcium carbonate, malic acid, cross linked povidone, microcrystalline cellulose, menthol, aspartame, magnesium stearate, and progesterone, wherein the formulation is in the form of an oral disintegration tablet or a dispersible tablet which produces a pH of more than 4.9 after disintegration in 900 mL of neutral pure water.

A composition of the invention comprises megestrol acetate, calcium carbonate, an acid selected from malic acid, maleic acid, succinic acid, tartaric acid, ascorbic acid, citric acid and mixtures thereof, cross linked povidone, wherein the formulation is in the form of an oral disintegration tablet or a dispersible tablet which produces a pH of more than 4.9 after disintegration in 900 mL of neutral pure water.

In an embodiment, the weakly effervescent disintegration formulation comprises calcium carbonate, malic acid, cross linked povidone, microcrystalline cellulose, menthol, aspartame, magnesium stearate, and megestrol acetate, wherein the formulation is in the form of an oral disintegration tablet or a dispersible tablet which produces a pH of more than 4.9 after disintegration in 900 mL of neutral pure water.

In an embodiment, the weakly effervescent disintegration formulation comprises a weak base selected from sodium bicarbonate and potassium bicarbonate; a weak acid selected from monosodium malate, monosodium maleate, monosodium succinate, monosodium adipate, monosodium oxalate, monosodium citrate, monosodium tartrate, monopotassium malate, monopotassium maleate, monopotassium succinate, monopotassium adipate, monopotassium oxalate, monopotassium citrate, monopotassium tartrate, and mixtures thereof; cross linked povidone, cross linked carboxymethyl sodium cellulose; and a dihydropyridine calcium channel blocker selected from amlodipine, L-amlodipine, felodipine, nitrendipine, benidipine, nifedipine, nimodipine and lacidipine, wherein the formulation is in the form of an oral disintegration tablet which produces a pH of more than 4.9 after disintegration in neutral pure water.

In an embodiment, the weakly effervescent disintegration formulation comprises sodium bicarbonate, monosodium tartrate, cross linked povidone, microcrystalline cellulose, menthol, aspartame, magnesium stearate, and amlodipine, wherein the formulation is in the form of an oral disintegration tablet which produces a pH of more than 4.9 after disintegration in neutral pure water.

In an embodiment, the weakly effervescent disintegration formulation comprises sodium bicarbonate, monosodium tartrate, cross linked povidone, microcrystalline cellulose, menthol, aspartame, magnesium stearate, and nitrendipine, wherein the formulation is in the form of an oral disintegration tablet which produces a pH of more than 4.9 after disintegration in neutral pure water.

In an embodiment, the weakly effervescent disintegration formulation comprises sodium bicarbonate, monosodium tartrate, cross linked povidone, microcrystalline cellulose, menthol, aspartame, magnesium stearate, and nimodipine, wherein the formulation is in the form of an oral disintegration tablet which produces a pH of more than 4.9 after disintegration in neutral pure water.

In an embodiment, the weakly effervescent disintegration formulation comprises sodium bicarbonate, monosodium tartrate, cross linked povidone, microcrystalline cellulose, menthol, aspartame, magnesium stearate, and nifedipine, wherein the formulation is in the form of an oral disintegration tablet which produces a pH of more than 4.9 after disintegration in neutral pure water.

In an embodiment, the weakly effervescent disintegration formulation comprises sodium bicarbonate, potassium bicarbonate, a weak acid selected from monosodium malate, monosodium maleate, monosodium succinate, monosodium adipate, monosodium oxalate, monosodium citrate, monosodium tartrate, monopotassium malate, monopotassium maleate, monopotassium succinate, monopotassium adipate, monopotassium oxalate, monopotassium citrate, monopotassium tartrate, and mixtures thereof; cross linked povidone, cross linked carboxymethyl sodium cellulose, and a sartan API selected from losartan, candesartan, valsartan, telmisartan, fimasartan, irbesartan and salts thereof; wherein the formulation is in the form of an oral disintegration tablet or a dispersible tablet which produces a pH of more than 4.9 after disintegration in neutral pure water.

In an embodiment, the weakly effervescent disintegration formulation comprises sodium bicarbonate, monosodium tartrate, cross linked carboxymethyl sodium cellulose, microcrystalline cellulose, menthol, aspartame, magnesium stearate, and valsartan, wherein the formulation produces a pH of more than 4.9 after disintegration in neutral pure water.

A composition of the invention comprises sodium bicarbonate, monosodium tartrate, cross linked povidone, microcrystalline cellulose, magnesium stearate and losartan potassium; wherein the formulation is in the form of an ODT or a dispersible tablet producing a pH of more than 4.9 after disintegration in 900 mL of neutral pure water.

Another composition of the invention comprises sodium bicarbonate, monosodium tartrate, cross linked carboxymethyl sodium cellulose, microcrystalline cellulose, magnesium stearate and irbesartan wherein the formulation is in the form of an ODT producing a pH of more than 4.9 after disintegration in 900 mL of neutral pure water.

Another composition of the invention comprises calcium carbonate, an acid selected from tartaric acid, malic acid, maleic acid, succinic acid, citric acid, cross linked carboxymethyl sodium cellulose, microcrystalline cellulose, magnesium stearate and irbesartan wherein the formulation is in the form of an ODT producing a pH of more than 4.9 after disintegration in neutral pure water.

In an embodiment, the weakly effervescent disintegration formulation comprises sodium bicarbonate, monosodium tartrate, cross linked povidone, microcrystalline cellulose, menthol, aspartame, magnesium stearate, and candesartan, wherein the formulation is in the form of an ODT which produces a pH of more than 4.9 after disintegration in neutral pure water.

In an embodiment, the weakly effervescent disintegration formulation comprises sodium bicarbonate, monosodium tartrate, cross linked povidone, cross linked carboxymethyl sodium cellulose, microcrystalline cellulose, menthol, aspartame, magnesium stearate, and telmisartan, wherein the formulation is in the form of an ODT which produces a pH of more than 4.9 after disintegration in neutral pure water.

In an embodiment, the weakly effervescent disintegration formulation comprises calcium carbonate; one acid selected from malic acid, maleic acid, succinic acid, adipic acid, fumaric acid, tartaric acid, and ascorbic acid; and a selective COX-2 inhibitor selected from celecoxib and imrecoxib, wherein the formulation is in the form of an ODT or a dispersible tablet which produces a pH of more than 4.9 after disintegration in neutral pure water.

Another composition of the invention comprises a selective COX-2 inhibitor selected from a group consisting of celecoxib and imrecoxib, one acid selected from a group consisting of malic acid, maleic acid, succinic acid, tartaric acid, ascorbic acid and citric acid and wherein the ratio of the weight of the acid to celecoxib or imrecoxib is from 1:1 to 6:1 and the formulation is in the form of a tablet, granules, or a capsule which has a dissolution rate of celecoxib or imrecoxib of more than 60% in 1000 mL of 0.25% sodium dodecyl sulfate of pH 7 sodium phosphate buffer solution under 50 RPM at 120 minutes after dissolution test; wherein the pH of the solution of disintegration is below 3.5.

In an embodiment, the weakly effervescent disintegration formulation comprises calcium carbonate, malic acid, cross linked carboxymethyl sodium cellulose, microcrystalline cellulose, menthol, aspartame, magnesium stearate, and celecoxib, wherein the formulation produces a pH of more than 4.9 after disintegration in neutral pure water.

In an embodiment, the weakly effervescent disintegration formulation comprises calcium carbonate, malic acid, cross linked carboxymethyl sodium cellulose, microcrystalline cellulose, menthol, aspartame, magnesium stearate, and imrecoxib, wherein the formulation produces a pH of more than 4.9 after disintegration in neutral pure water.

In another composition of the celecoxib, each formulation comprises 65 mg to 200 mg celecoxib, 65 mg to 960 mg of an organic acid selected from the group consisting of ascorbic acid, malic acid, maleic acid, succinic acid, tartaric acid and citric acid or a mixture thereof, 50 mg to 160 mg cross linked povidone, 50 mg to 160 mg cross linked carboxymethyl sodium cellulose, magnesium stearate and a small amount of PVPK30; wherein the composition is in the form of granules, capsule or tablets which has a dissolution rate of more than 60% of celecoxib in 1000 mL of pH 7 sodium phosphate buffer solution containing 2.5 g of sodium dodecyl sulfate under 50 RPM at 120 minutes after dissolution test.

In another composition of the invention of celecoxib, each dosage comprises 65 mg to 200 mg celecoxib, 65 mg to 960 mg of an acid selected from ascorbic acid, malic acid, maleic acid, succinic acid, tartaric acid and citric acid or a mixture thereof, calcium carbonate, cross linked povidone, cross linked carboxymethyl sodium cellulose, magnesium stearate and a small amount of PVPK30; wherein the formulation is in the form of a granule or tablets which has a dissolution rate of more than 60% of celecoxib and calcium ions in a 1000 mL of pH 7 sodium phosphate buffer solution containing 2.5 g of sodium dodecyl sulfate under 50 RPM at 120 minutes after dissolution test;

In the composition of the invention of imrecoxib, each dosage comprises 40 mg to 80 mg imrecoxib, 40 mg to 480 mg of an acid selected from ascorbic acid, malic acid, maleic acid, succinic acid, tartaric acid and citric acid or a mixture thereof, cross linked povidone, cross linked carboxymethyl sodium cellulose, wherein the formulation is in the form of granules, a capsule, or a tablet.

In an embodiment, the weakly effervescent disintegration formulation comprises a bicarbonate salt, a monosodium salt of a binary acid, cross linked carboxymethyl sodium cellulose, microcrystalline cellulose, menthol, aspartame, magnesium stearate, and etoricoxib, wherein the formulation produces a pH of more than 4.9 after disintegration in neutral pure water.

In an embodiment, the weakly effervescent disintegration formulation comprises calcium carbonate; an acid selected from malic acid, maleic acid, adipic acid, fumaric acid, tartaric acid, succinic acid, ascorbic acid, citric acid and mixtures thereof; cross linked povidone, cross linked carboxymethyl sodium cellulose, and a serotonin reuptake inhibitor selected from fluoxetine, paroxetine, sertraline, fluvoxamine, citalopram, escitalopram, and salts thereof, wherein the formulation produces a pH of more than 4.9 after disintegration in neutral pure water.

In an embodiment, the weakly effervescent disintegration formulation comprises calcium carbonate, malic acid, cross linked povidone, microcrystalline cellulose, menthol, aspartame, magnesium stearate, and fluoxetine hydrochloride, wherein the formulation is in the form of an oral disintegration tablet which produces a pH of more than 4.9 after disintegration in neutral pure water.

In an embodiment, the weakly effervescent disintegration formulation comprises calcium carbonate, malic acid, cross linked povidone, microcrystalline cellulose, menthol, aspartame, magnesium stearate and paroxetine HCl, wherein the formulation is in the form of an oral disintegration tablet which produces a pH of more than 4.9 after disintegration in neutral pure water.

In an embodiment, the weakly effervescent disintegration formulation comprises calcium carbonate, malic acid, cross linked povidone, microcrystalline cellulose, menthol, aspartame, magnesium stearate, and fluvoxamine, wherein the formulation produces a pH of more than 4.9 after disintegration in neutral pure water.

In an embodiment, the weakly effervescent disintegration formulation comprises sodium bicarbonate, potassium bicarbonate; an acid selected from monosodium malate, monosodium maleate, monosodium succinate, monosodium adipate, monosodium oxalate, monosodium citrate, monosodium tartrate, monopotassium malate, monopotassium maleate, monopotassium succinate, monopotassium adipate, monopotassium oxalate, monopotassium tartrate and mixtures thereof; cross linked povidone, cross linked carboxymethyl sodium cellulose, and an antifibrinolytic drug selected from 6-aminocaproic acid, tranexamic acid and hydroxybenzylamine, wherein the formulation produces a pH of more than 4.9 after disintegration in neutral pure water.

In an embodiment, the weakly effervescent disintegration formulation comprises sodium bicarbonate, monosodium tartrate, cross linked carboxymethyl sodium cellulose, microcrystalline cellulose, menthol, aspartame, magnesium stearate, and tranexamic acid, wherein the formulation produces a pH of more than 4.9 after disintegration in neutral pure water.

In an embodiment, the weakly effervescent disintegration formulation comprises sodium bicarbonate, monosodium tartrate, cross linked carboxymethyl sodium cellulose, microcrystalline cellulose, menthol, aspartame, magnesium stearate, and 6-aminocaproic acid, wherein the formulation produces a pH of more than 4.9 after disintegration in neutral pure water.

In an embodiment, the weakly effervescent disintegration formulation comprises sodium bicarbonate, monosodium tartrate, cross linked carboxymethyl sodium cellulose, microcrystalline cellulose, menthol, aspartame, magnesium stearate and hydroxybenzylamine, wherein the formulation produces a pH of more than 4.9 after disintegration in neutral pure water.

Another composition of the invention comprises tranexamic acid, sodium bicarbonate, potassium bicarbonate, an acid selected from monosodium malate, monosodium maleate, monosodium succinate, monosodium citrate, monosodium tartrate, monopotassium malate, monopotassium maleate, monopotassium succinate, monopotassium tartrate, cross linked carboxymethyl sodium cellulose, and cross linked povidone, wherein the formulation is in the form of an oral disintegration tablet or a dispersible tablet which produces a pH of more than 4.9 after disintegration in neutral pure water.

In an embodiment, the weakly effervescent disintegration formulation comprises calcium carbonate; an acid selected from malic acid, maleic acid, succinic acid, adipic acid, fumaric acid, tartaric acid, ascorbic acid and mixtures thereof; cross linked povidone, cross linked carboxymethyl sodium cellulose, and an antihistamine selected from cetirizine, levocetirizine, hydroxyzine, promethazine, fexofenadine, loratadine, terfenadine, desloratadine, chlorpheniramine, dexchlorpheniramine, clemastine, triprolidine and diphenhydramine or a salt thereof, wherein the formulation is in the form of an oral disintegration tablet which produces a pH of more than 4.9 after disintegration in neutral pure water.

In an embodiment, the weakly effervescent disintegration formulation comprises calcium carbonate, malic acid, cross linked povidone, microcrystalline cellulose, menthol, aspartame, magnesium stearate and loratadine, wherein the formulation is in the form of an oral disintegration tablet which produces a pH of more than 4.9 after disintegration in neutral pure water.

In an embodiment, the weakly effervescent disintegration formulation comprises calcium carbonate, malic acid, cross linked povidone, microcrystalline cellulose, menthol, aspartame, magnesium stearate and clemastine, wherein the formulation is in the form of an oral disintegration tablet which produces a pH of more than 4.9 after disintegration in neutral pure water.

In an embodiment, the weakly effervescent disintegration formulation comprises calcium carbonate, malic acid, cross linked povidone, microcrystalline cellulose, menthol, aspartame, magnesium stearate and cetirizine, wherein the formulation is in the form of an oral disintegration tablet which produces a pH of more than 4.9 after disintegration in neutral pure water.

In an embodiment, the weakly effervescent disintegration formulation comprises calcium carbonate, malic acid, cross linked povidone, microcrystalline cellulose, menthol, aspartame, magnesium stearate and olanzapine, wherein the formulation is in the form of an oral disintegration tablet which produces a pH of more than 4.9 after disintegration in neutral pure water.

In an embodiment, the weakly effervescent disintegration formulation comprises calcium carbonate, malic acid, cross linked povidone, microcrystalline cellulose, menthol, aspartame, magnesium stearate and quetiapine, wherein the formulation produces a pH of more than 4.9 after disintegration in neutral pure water.

In an embodiment, the weakly effervescent disintegration formulation comprises calcium carbonate, malic acid, cross linked povidone, microcrystalline cellulose, menthol, aspartame, magnesium stearate and paliperidone, or a salt thereof, wherein the formulation produces a pH of more than 4.9 after disintegration in neutral pure water.

In an embodiment, the weakly effervescent disintegration formulation comprises calcium carbonate; an acid selected from malic acid, maleic acid, succinic acid, adipic acid, fumaric acid, tartaric acid, ascorbic acid, and mixtures thereof; cross linked povidone, cross linked carboxymethyl sodium cellulose; and a triptan drug selected from rizatriptan, sumatriptan, zolmitriptan, naratriptan, almotriptan, eletriptan and frovatriptan, wherein the formulation is in the form of an oral disintegration tablet or a dispersible tablet and produces a pH of more than 4.9 after disintegration in neutral pure water.

In an embodiment, the weakly effervescent disintegration formulation comprises calcium carbonate, maleic acid, cross linked povidone, microcrystalline cellulose, menthol, aspartame, magnesium stearate and rizatriptan, wherein the formulation is in the form of an oral disintegration tablet which produces a pH of more than 4.9 after disintegration in neutral pure water.

In an embodiment, the weakly effervescent disintegration formulation comprises calcium carbonate, malic acid, cross linked povidone, microcrystalline cellulose, menthol, aspartame, magnesium stearate and sumatriptan, wherein the formulation produces a pH of more than 4.9 after disintegration in neutral pure water.

In an embodiment, the weakly effervescent disintegration formulation comprises calcium carbonate, malic acid, cross linked povidone, microcrystalline cellulose, menthol, aspartame, magnesium stearate and zolmitriptan, wherein the formulation produces a pH of more than 4.9 after disintegration in neutral pure water.

In another composition of the present invention, it comprises calcium carbonate, an acid selected from malic acid, maleic acid, succinic acid, tartaric acid, ascorbic acid and citric acid, cross linked povidone, cross linked carboxymethyl sodium cellulose and sildenafil citrate wherein the formulation is in the form of an oral disintegration tablet or a dispersible tablet and produces a pH of more than 4.9 after disintegration in neutral pure water.

In another aspect the invention relates to a method of preparing a weakly effervescent fast disintegration formulation comprising: preparing acidic granules by mixing a weak acid selected from monosodium malate, monosodium maleate, monosodium succinate, monosodium adipate, monosodium oxalate, monosodium citrate, monosodium tartrate, monopotassium malate, monopotassium maleate, monopotassium succinate, monopotassium adipate, monopotassium oxalate, monopotassium citrate, or monopotassium tartrate, with an acidic or neutral API, filler and optionally an expander in a fluid bed or granulator; drying the acidic granules; mixing the dried acidic granules with a weak base selected from sodium bicarbonate, potassium bicarbonate, lithium bicarbonate and calcium carbonate powder or granules, and optionally filler, lubricant, sweeting agent and peppermint; compressing the mixture to form a weakly effervescent fast disintegration formulation, which has a pH of more than 4.9 after disintegration in neutral pure water, preferably 200 mL of neutral pure water.

A method of preparing the composition of the celecoxib comprising:
1. Mixing 1 part of celecoxib with 1-6 parts of an organic acid selected from the group consisting of ascorbic acid, malic acid, maleic acid, succinic acid, tartaric acid and citric acid or a mixture thereof, with an alcohol selected from ethanol and benzyl alcohol until the celecoxib and the organic acid is fully dissolved.
2. 1 part of cross linked povidone, 1 part of cross linked sodium carboxymethyl cellulose is place in a fluid bed or granulator.
3. The alcoholic solution containing celecoxib and organic acid is sprayed into the fluid bed.
4. A small amount of aqueous 8% PVPK30 is sprayed into the fluid bed until the size of granules is about 24 mesh. The granules made are dried by hot air; and the granules are mixed with magnesium stearate and are compressed into a tablet or filled into a capsule or granule bag.

In another aspect the invention also relates to a method of preparing a weakly effervescent disintegration formulation comprising: preparing weakly basic granules by mixing a weak base selected from calcium carbonate, potassium carbonate, sodium carbonate with a basic or neutral API, filler and optionally an expander in a fluid bed or granulator; drying the granules; mixing the dried granules with a weak acid and optionally filler, lubricant, sweeting agent and peppermint; compressing the mixture to form a weakly effervescent disintegration formulation, which has a pH of more than 4.9 after disintegration in neutral pure water.

A method of preparing a weakly effervescent disintegration formulation comprising: preparing acidic granules by mixing a weak acid selected from monosodium malate, monosodium maleate, monosodium succinate, monosodium adipate, monosodium oxalate, monosodium citrate, monosodium tartrate, monopotassium malate, monopotassium maleate, monopotassium succinate, monopotassium adipate, monopotassium oxalate, monopotassium citrate, monopotassium tartrate, with or without an acidic or neutral API, filler and optionally an expander in a fluid bed or granulator; drying the acidic granules; preparing basic granules by mixing a weak base selected from sodium bicarbonate, potassium bicarbonate, lithium bicarbonate and calcium carbonate, with or without basic or neutral API, filler and optionally an expander in a fluid bed or granulator; drying the acidic granules; mixing the dried acidic granules with a weak base granules, and optionally filler, lubricant, sweeting agent and peppermint; compressing the mixture to form a weakly effervescent formulation, which further has a pH of more than 4.9 after disintegration in neutral pure water.

### OVERVIEW OF THE INVENTION

The composition of the present invention contains a water insoluble API selected from celecoxib, imrecoxib, calcium carbonate, an organic acid selected from ascorbic acid, malic acid, maleic acid, succinic acid, tartaric acid and citric acid or a mixture thereof, cross linked povidone, cross linked sodium carboxymethyl cellulose, magnesium stearate or PEG 6000. This composition may be in the form of granules, a capsule, a tablet or a dispersible tablet which has high rate of dissolution of API contained in aqueous medium.

The present invention broadly relates to a convenient drug delivery system, and methods of producing a drug delivery system, preferably using common excipients via a simple method and at low cost. More particularly, the present invention relates to rapid disintegrating dosage forms, such as weakly effervescent tablets or granules, such as orally disintegrating tablets (ODTs) and dispersible tablets, and methods for their production and administration. The weakly effervescent formulations according to the present invention are suitable for the oral delivery or administration of APIs such as a wide range of pharmaceutical drugs or food nutrients, such as vitamins or minerals.

Another composition of the present invention contains celecoxib, an organic acid selected from the group consisting of ascorbic acid, malic acid, maleic acid, succinic acid, tartaric acid and citric acid or a mixture thereof, cross linked povidone, cross linked sodium carboxymethyl cellulose, and magnesium stearate. This formulation has high rate of dissolution of celecoxib in aqueous medium. The higher dissolution of celecoxib of the present invention compared with celecoxib capsules on the market will reduce the amount of celecoxib needed to treat the joint pain; this in turn will reduce the alimentary side effects of celecoxib. The ratio of the weight of an organic acid to celecoxib is in the range from 1:1 to 6:1, the preferred range is from 2:1 to 5:1, by weight.

Another composition of the present invention contains imrecoxib, an organic acid selected from the group including ascorbic acid, malic acid, maleic acid, succinic acid, tartaric acid and citric acid or a mixture thereof, cross linked povidone, cross linked sodium carboxymethyl cellulose, and magnesium stearate. This formulation has high rate of dissolution of imrecoxib in aqueous medium. The higher dissociation of imrecoxib of the present invention will reduce the amount of imrecoxib needed to treat the pain; this in turn will reduce the alimentary side effect of imrecoxib.

The composition of the invention contains the celecoxib, calcium carbonate and an organic acid selected from the group including ascorbic acid, malic acid, maleic acid, succinic acid, tartaric acid and citric acid, the dissociation of calcium ions in the alimentary system will be increased. More particularly, the present invention containing celecoxib or imrecoib, calcium carbonate and an organic acid will increase the dissociation of calcium ions and the dissolution of celecoxib or imrecoib in aqueous medium or alimentary system.

Another composition of the present invention contains ascorbic acid, calcium carbonate, cross-linked povidone, cross linked sodium carboxymethyl cellulose, and magnesium stearate. This formulation may be in the form of granules, a capsule, a tablet or an oral disintegration tablet which has high rate of dissolution of calcium ions in neutral pure water. The higher dissociation of calcium ions of the present invention compared with a calcium carbonate tablet on the market will reduce the amount of calcium carbonate needed to treat the joint pain or hypocalcemia; this in turn will reduce the alimentary side effects of calcium carbonate such as constipation. The ratio of the ascorbic acid to calcium carbonate is in the range from 1:1 to 3:1 by weight/weight ratio. After the formulation disintegrates in 200 mL of neutral pure water, the pH range of the suspension formed is greater than 4.9. The dissolution of the calcium ions is about 20-65% in 900 mL of neutral pure water under stirring at a speed of 75 RPM when measured at 30 min.

Embodiments of the present invention relate to a weakly effervescent formulation comprising a carbonate salt and weak organic acid. In a preferred embodiment, the weakly effervescent formulation comprises a carbonate salt, and a weak organic acid, cross linked povidone, cross linked sodium carboxymethyl cellulose, microcrystalline cellulose, magnesium stearate or PEG 6000. After the weakly effervescent disintegrating formulation of carbonate disintegrates in neutral pure water, the pH of the suspension formed is greater than 4.9, such that the formulation may be in the form of an oral disintegration tablet (ODT) or a dispersible tablet.

The composition of the present invention contains an organic acid selected from the group including ascorbic acid, malic acid, maleic acid, succinic acid, tartaric acid and citric acid or a mixture thereof, calcium carbonate, cross linked povidone, and magnesium stearate. This formulation may be in the form of granules, a capsule, a tablet, an oral disintegration tablet or a dispersible tablet which has high rate of dissociation of calcium ions in neutral aqueous medium. The higher dissociation of calcium ions of the present invention compared with calcium carbonate tablet on the market will reduce the amount of calcium carbonate needed to treat the hypocalcemia; this in turn will reduce the alimentary side effects of calcium carbonate such as constipation. The ratio of the binary acid to calcium carbonate is in the range from 0.15:1 to 1:1 by molar ratio. After the formulation disintegrates in 900 mL of neutral pure water, the pH of the suspension formed is greater than 4.9. The dissolution of the calcium ions of the invention of calcium carbonate is about 20-75% in 900 mL of neutral pure water under stirring at a speed of 75 RPM when measured at 30 min.

The composition of the present invention contains calcium carbonate, a malic acid, cross linked povidone, and magnesium stearate or PEG 6000. This formulation may be in the form of granules, a capsule, a tablet, or an oral disintegration tablet which has a dissolution of calcium ions of about 20-75% in 900 mL of neutral pure water under stirring at a speed of 75 RPM when measured at 30 min; wherein the pH of the suspension formed is greater than 4.9.

Another composition of the present invention contains calcium carbonate, a maleic acid, cross linked povidone, and magnesium stearate. This formulation may be in the form of granules, a capsule, a tablet, or an oral disintegration tablet which has a dissolution of calcium ions of about 20-75% in 900 mL of neutral pure water under stirring at a speed of 75 RPM when measured at 30 min; wherein the pH of the suspension formed is greater than 4.9.

Another composition of the present invention contains calcium carbonate, a succinic acid, cross linked povidone, and magnesium stearate. This formulation may be in the form of granules, a capsule, a tablet, an oral disintegration tablet or a dispersible tablet which has a dissolution of calcium ions of about 20-75% in 900 mL of neutral pure water under stirring at a speed of 75 RPM when measured at 30 min; wherein the pH of the suspension formed is greater than 4.9.

Another composition of the present invention contains calcium carbonate, a tartaric acid, cross linked povidone, and magnesium stearate. This formulation may be in the form of granules, a capsule, a tablet, an oral disintegration tablet or a dispersible tablet which has a dissolution of calcium ions of about 20%-75% in 900 mL of neutral pure water under stirring at a speed of 75 RPM when measured at 30 min; wherein the pH of the suspension formed is greater than 4.9.

Another composition of the present invention contains calcium carbonate, a citric acid, cross linked povidone, and magnesium stearate or PEG 6000. This formulation may be in the form of granules, a capsule, a tablet, an oral disintegration tablet or a dispersible tablet which has a dissolution of calcium ions of about 20-75% in 900 mL of neutral pure water under stirring at a speed of 75 RPM when measured at 30 min; wherein the pH of the suspension formed is greater than 4.9.

Another composition of the present invention contains calcium carbonate, an ascorbic acid, cross-linked povidone, and magnesium stearate or PEG 6000. This formulation may be in the form of granules, a capsule, a tablet, an oral disintegration tablet or a dispersible tablet which has a dissolution of calcium ions of about 20-65% in 900 mL of neutral pure water under stirring at a speed of 75 RPM when measured at 30 min; wherein the pH of the suspension formed is greater than 4.9.

Another composition of the present invention contains calcium carbonate, a glucosamine sulfate, cross linked povidone, and magnesium stearate. This formulation may be in the form of granules or tablet which has a dissolution of calcium ions of about 35-55% in 900 mL of neutral pure water under stirring at a speed of 75 RPM when measured at 30 min; wherein the pH of the suspension formed is from 6.5 to 7.5.

Another composition of the present invention contains 25 mg to 150 mg calcium carbonate, 250 mg to 1500 mg of glucosamine hydrochloride, cross linked povidone, and magnesium stearate. This formulation may be in the form of granules, a capsule, or a tablet which has a dissolution of calcium ions of about 35-55% in 900 mL of neutral pure water under stirring at a speed of 75 RPM when measured at 30 min; wherein the pH of the suspension formed is from 6.5 to 7.5.

Another weakly effervescent disintegration formulation of the invention comprises sodium bicarbonate, glucosamines sulfate, cross linked povidone, and magnesium stearate, wherein the formulation is in the form of granules, a capsule or a dispersible tablet and has a pH ranging from 6.5 to 7.5 in 200 mL of neutral water.

Another weakly effervescent disintegration formulation of the invention comprises 1 part of glucosamine hydrochloride, 0.1 to 1 parts of sodium bicarbonate, 0.2-0.4 parts of cross linked povidone, and magnesium stearate, wherein the formulation is in the form of granules or a dispersible tablet and has a pH ranging from 6.5 to 7.5 in 200 mL of neutral water.

Advantageously, by achieving a pH greater than 4.9, for example a pH from 4.9 to 7.5, or a pH from 4.9 to 7.0, or a pH from 5 to 7 after dissolution, the orally disintegrating tablets or dispersible tablets according to the present invention do not burn the oral cavity.

Other embodiments of the present invention relate to a weakly effervescent formulation of calcium carbonate and an organic acid. In a preferred embodiment, the weakly effervescent formulation comprises calcium carbonate and a weak organic acid, cross linked povidone, cross linked sodium carboxymethyl cellulose, microcrystalline cellulose, and magnesium stearate or PEG600.

The weak organic acid may be a monocarboxylic acid or a dicarboxylic acid (also referred to herein as a binary acid). In preferred embodiments, the organic acid is a dicarboxylic acid. In preferred embodiments, the organic acid is selected from malic acid, maleic acid, succinic acid, adipic acid, fumaric acid, tartaric acid, ascorbic acid and citric acid. In particularly preferred embodiments, the organic acid is malic acid.

By modulating the relative amount of calcium carbonate and amount of organic acid we may modulate the final pH of the solution of disintegration to pH 5-7, burning of the mouth may be prevented, and the dissociation of calcium ions to 20-75% may be controlled. The high dissociation of calcium ions will lead to good absorption of the calcium and reduced strength and dosage per day consumed to maintain normal blood calcium level.

The weakly effervescent formulations according to the present invention comprise a pair of weakly effervescent chemicals and an API. The effervescent chemical pair comprises a weak acid/base pair. The acid/base pair is specified or restricted such that the pH of the resultant solution when the formulation is added to water, is greater than 4.9. In an embodiment, the weakly effervescent chemical pair is calcium carbonate with a binary acid. In another embodiment the weakly effervescent chemical pair is sodium or potassium bicarbonate with the monosodium salt of a binary acid, such as malate or malic acid. The formulations may also comprise an API such as a pharmaceutical drug or a food nutrient, such as one or more vitamins and minerals.

The formulations according to the present invention may comprise a range of APIs from a variety of drug classes, including for example hemostatic drugs, antihypertensive drugs, antiemetic drugs, sartans, hypoglycemic drugs, anti-allergic drugs, anti-impotence drugs, contraceptives, antidepressants, and COX-2 inhibitors

According to embodiments of the present invention the time of disintegration of the calcium carbonate is typically less than 3 minutes in a small amount of neutral (preferably pure) water, for example, volumes of about 100-300 mL, preferably about 200 mL. Due to the favourable disintegration time, such formulations advantageously may prevent or mitigate the risk of asphyxia, even for large tablets, such as tablets greater than 1 gram. After the weakly effervescent disintegrating formulation disintegrates in neutral pure water, e.g., about 200 mL neutral pure water, the pH of the suspension or solution formed is greater than 4.9, such that the formulation may be in the form of an oral disintegration tablet or a dispersible tablet.

An embodiment of the present invention relates to a weakly effervescent formulation comprising calcium carbonate and malic acid. In a preferred embodiment, the weakly effervescent formulation comprises calcium carbonate, malic acid, cross linked povidone, cross linked sodium carboxymethyl cellulose, microcrystalline cellulose, magnesium stearate and PEG 6000. In preferred embodiments, the mole ratio of malic acid to calcium carbonate is in the range from 0.15:1.0 or from 0.35:1.0 to 1.0:1.0. After the formulation disintegrates in neutral pure water, for example, a volume of neutral pure water of about 100-300 mL, preferably about 200 mL, the pH range of the suspension or solution formed is greater than 4.9, preferably from 5 to 7, such that the formulation may be in the form of an oral disintegration tablet or a dispersible tablet. The dissolution of the calcium in the weakly effervescent formulation may be more than 20% and less than 75% in 900 mL of neutral pure water under stirring at a speed of 75 RPM when measured at 30 min.

The amount of calcium carbonate contained in each weakly effervescent formulation, for example, weakly effervescent tablet formulation is less than 150 mg. In a preferred embodiment, the amount of calcium carbonate in the weakly effervescent tablet formulation is greater than 20 mg. In a preferred embodiment, the amount of calcium carbonate contained in weakly effervescent tablet formulation for adults is from about 40 mg to about 125 mg calcium carbonate per tablet, for example, about 75 mg calcium carbonate per tablet. Preferably, such weakly effervescent tablet formulations have a dissolution of calcium ions in the range of 20% to 75%. The preferred daily dosage of calcium carbonate for an adult is typically in the range of 40 mg to 80 mg per day. The daily dosage of calcium carbonate for a child is typically in the range of 20 mg to 75 mg per day.

Another embodiment of the present invention relates to a weakly effervescent formulation which comprises calcium carbonate and tartaric acid. In a preferred embodiment, the weakly effervescent formulation comprises calcium carbonate, tartaric acid, cross linked povidone, cross linked sodium carboxymethyl cellulose, microcrystalline cellulose, magnesium stearate and PEG 6000. In preferred embodiments, the mole ratio of tartaric acid to calcium carbonate is in the range from 0.35:1.0 to 1.0:1.0. After the weakly effervescent disintegrating formulation disintegrates in neutral pure water, the pH range of the suspension or solution formed is from 5 to 7, such that the formulation may be in the form of a capsule, granules, an oral disintegration tablet or a dispersible tablet. The dissolution of the calcium in the weakly effervescent formulation may be more than 20% and in the range of from 20-75% in 900 mL of neutral pure water under stirring at a speed of 75 RPM when measured at 30 min. In preferred embodiments, the amount of calcium carbonate contained in each tablet may be less than 150 mg. In a preferred embodiment, the dose for adults is from 50 mg to 125 mg calcium carbonate per tablet with a dissolution of calcium ions in the range of 20% to 75%. In preferred embodiments, the dosage for an adult is in the range of 50 mg to 125 mg per day. In preferred embodiments, the dosage for a child is in the range of 20 mg to 100 mg per day. In particularly preferred embodiments, the range of amount of calcium carbonate contained in each tablet is from 20 mg to 75 mg. In particularly preferred embodiments the dosage for an adult is in the range of 40 mg to 80 mg per day. In particularly preferred embodiments the dosage for a child is in the range of 20 mg to 75 mg per day.

Another embodiment of the present invention relates to a weakly effervescent formulation which comprises calcium carbonate and succinic acid. In a preferred embodiment, the weakly effervescent formulation comprises calcium carbonate, succinic acid, cross linked povidone, cross linked sodium carboxymethyl cellulose, microcrystalline cellulose, magnesium stearate and PEG 6000. In preferred embodiments, the mole ratio of succinic acid to calcium carbonate is in the range from 0.35:1.0 to 1.0:1.0. After the weakly effervescent disintegrating formulation disintegrates in neutral pure water, for example, a volume of neutral pure water of about 100-300 mL, preferably about 200 mL, the pH of the suspension or solution formed is more than 4.9, such that the formulation may be in the form of in the form of granules, a capsule, a tablet, an oral disintegration tablet or a dispersible tablet. The dissolution of the calcium in the weakly effervescent formulation may be more than 20% in 900 mL of neutral pure water under stirring at a speed of 75 RPM when measured at 30 min. In preferred embodiments, the amount of calcium carbonate contained in each tablet may be less than 150 mg. In a preferred embodiment, the dose for adults is from 50 mg to 125 mg calcium carbonate per tablet with a dissolution of calcium ions in the range of 20% to 75%. In preferred embodiments, the dosage for an adult is in the range of 50 mg to 125 mg per day. In preferred embodiments, the dosage for a child is in the range of 20 mg to 100 mg per day.

Another embodiment of the present invention relates to a weakly effervescent formulation which comprises calcium carbonate and adipic acid. In a preferred embodiment, the weakly effervescent formulation comprises calcium carbonate, adipic acid, cross linked povidone, cross linked sodium carboxymethyl cellulose, microcrystalline cellulose, magnesium stearate and PEG 6000. The mole ratio of adipic acid to calcium carbonate is preferably in the range from 0.35:1.0 to 1.0:1.0. After the weakly effervescent disintegrating formulation disintegrates in neutral pure water e.g., 200 mL of neutral pure water, the pH of the suspension or solution formed is more than 4.9, such that the formulation may be in the form of an oral disintegration tablet or a dispersible tablet. The dissolution of the calcium in the weakly effervescent formulation may be more than 20% in 900 mL of neutral pure water under stirring at a speed of 75 RPM when measured at 30 min. In preferred embodiments, the amount of calcium carbonate contained in each tablet may be less than 150 mg. In a preferred embodiment, the dose for adults is from 50 mg to 125 mg calcium carbonate per tablet with a dissolution of calcium ions in the range of 20% to 75%. In preferred embodiments, the dosage for an adult is in the range of 50 mg to 125 mg per day. In preferred embodiments, the dosage for a child is in the range of 20 mg to 100 mg per day.

Another embodiment of the present invention relates to a weakly effervescent formulation which comprises calcium carbonate and fumaric acid. In a preferred embodiment, the weakly effervescent formulation comprises calcium carbonate, fumaric acid, cross linked povidone, cross linked sodium carboxymethyl cellulose, microcrystalline cellulose, magnesium stearate and PEG 6000. The mole ratio of fumaric acid to calcium carbonate is preferably in the range from 0.35:1.0 to 1.0:1.0. After the weakly effervescent disintegrating formulation disintegrates in the neutral pure water, e.g., 200 mL of neutral pure water, the pH of the suspension or solution formed is greater than 4.9, such that the formulation may be in the form of an oral disintegration tablet or a dispersible tablet. The dissolution of the calcium in the weakly effervescent formulation may be more than 20% in 900 mL of neutral pure water under stirring at a speed of 75 RPM when measured at 30 min. In preferred embodiments, the amount of calcium carbonate contained in each tablet may be less than 150 mg. In a preferred embodiment, the dose for adults is from 50 mg to 125 mg calcium carbonate per tablet with a dissolution of calcium ions in the range of 20% to 75%. In preferred embodiments the dosage for an adult is in the range of 50 mg to 125 mg per day. In preferred embodiments, the dosage for a child is in the range of 20 mg to 100 mg per day.

Another embodiment of the present invention relates to a weakly effervescent formulation comprising calcium carbonate and ascorbic acid. In a preferred embodiment, the weakly effervescent formulation comprises calcium carbonate, ascorbic acid, cross linked povidone, cross linked sodium carboxymethyl cellulose, microcrystalline cellulose, magnesium stearate and PEG 6000. The ratio of calcium carbonate to ascorbic acid is preferably in a ratio of about 1.0:1.0 to about 1.0:3.0 by weight. After the weakly effervescent disintegrating formulation disintegrates in neutral pure water, e.g., 200 mL of neutral pure water, the pH of the suspension or solution formed is greater than 4.9 and less than 7, such that the formulation may be in the form of a capsule, a tablet, an oral disintegration tablet or a dispersible tablet or granules. The dissolution of the calcium in the weakly effervescent formulation may be more than 20% and in the range of 20-65% in 900 mL of neutral pure water under stirring at a speed of 75 RPM when measured at 30 min.

In accordance with a preferred embodiment, the amount of calcium carbonate contained per tablet is in the range of 20 mg to 125 mg, preferably in the range of 20 mg to 100 mg, more preferably in the range of 20 mg to 75 mg; the dissolution of the calcium ions is in the range of 20% to 75%; and the pH of the final solution or suspension resulting from the dissolution of a calcium carbonate tablet, dispersible tablet or ODT is from pH 5 to 7 in 200 mL of neutral pure water.

Vitamins and minerals are food supplements which include vitamin B1, vitamin B2, vitamin B6, vitamin B12, vitamin C, vitamin A, vitamin D, vitamin E, vitamin K, folic acid, nicotinic acid, pantothenic acid, choline, biotin, calcium carbonate, iron sulfate, copper sulfate, zinc sulfate, sodium chloride, magnesium sulfate, potassium chloride. In an embodiment, the present invention enables such food supplements to be formulated as a weakly effervescent disintegrating formulation e.g., an orodispersible or orally disintegrating tablet or granules comprising (or consisting essentially of) the food supplements, a pair of weak acid and a carbonate, and one or more excipients selected from filling agents, a diluent, lubricants and expanders. After the weakly effervescent disintegrating formulation of food supplement disintegrates in the neutral pure water, the pH of the suspension or solution formed is greater than 4.9.

Another embodiment of the present invention relates to a weakly effervescent formulation which comprises calcium carbonate, ascorbic acid and a glucosamine salt selected from glucosamine chloride and glucosamine sulfate. In a preferred embodiment, the weakly effervescent formulation comprises calcium carbonate, ascorbic acid, a glucosamine salt selected from glucosamine chloride and glucosamine sulfate, cross linked povidone, cross linked sodium carboxymethyl cellulose, microcrystalline cellulose, magnesium stearate and PEG 6000. The ratio of calcium carbonate to ascorbic acid is preferably 1:1 to 1:3 by weight. After the weakly effervescent disintegrating formulation disintegrates in neutral pure water, e.g., 200 mL of neutral pure water, the pH of the suspension or solution formed is more than 4.9, such that the formulation is in the form of a tablet or a dispersible tablet. The dissolution of the calcium in the weakly effervescent formulation may be more than 20% and is in the range of 20% to 80% in 900 mL of neutral pure water under stirring at a speed of 75 RPM when measured at 30 min.

Another embodiment relates to formulations comprising calcium carbonate, ascorbic acid and a glucosamine salt selected from glucosamine chloride and glucosamine sulfate. In a preferred embodiment, the amount of calcium carbonate in each tablet is from 15 mg to 75 mg, the amount of ascorbic in each tablet is in the range of 15 mg to 225 mg, the amount of glucosamine hydrochloride is in the range of 300 mg to 750 mg, or the amount of glucosamine sulfate is in the range of 386 mg to 965 mg, and dissolution of the calcium ions is in the range of 20% to 75% or 20% to 80% in 900 mL of neutral water.

Another embodiment of the present invention relates to a weakly effervescent formulation which comprises calcium carbonate, ascorbic acid and glucosamine hydrochloride. In a preferred embodiment, the weakly effervescent formulation comprises calcium carbonate, ascorbic acid, glucosamine hydrochloride, cross linked povidone, cross linked sodium carboxymethyl cellulose, microcrystalline cellulose, magnesium stearate and PEG 6000. The ratio of calcium carbonate to ascorbic acid is preferably from 1:1 to 1:3 by weight. After the weakly effervescent disintegrating formulation disintegrates in neutral pure water, e.g., 200 mL of neutral pure water, the pH of the suspension or solution formed is from 4.9 to 7.0, such that the formulation is in the form of a dispersible tablet or tablet. In addition, the dissolution of the calcium in the weakly effervescent formulation may be more than 20% and in the range of 20% to 80% in 900 mL of neutral pure water under stirring at a speed of 75 RPM when measured at 30 min.

Another embodiment of the present invention relates to a weakly effervescent formulation which comprises calcium carbonate, ascorbic acid and glucosamine sulfate. In a preferred embodiment, the weakly effervescent formulation comprises calcium carbonate, ascorbic acid and glucosamine sulfate, cross linked povidone, cross linked sodium carboxymethyl cellulose, microcrystalline cellulose, magnesium stearate and PEG 6000. The ratio of calcium carbonate to ascorbic acid is preferably from 1:1 to 1:3 by weight. After the weakly effervescent disintegrating formulation disintegrates in neutral pure water, e.g., 200 mL of neutral pure water, the pH of the suspension or solution formed is greater than 4.9 and the dissolution of the calcium ions is in the range of 40% to 80% under stirring at a speed of 75 RPM when measured at 30 min and the formulation may be granules, a tablet or a dispersible tablet.

Another embodiment of the present invention relates to a weakly effervescent formulation which comprises calcium carbonate, ascorbic acid, a glucosamine salt and vitamin D3. In a preferred embodiment, the weakly effervescent disintegration formulation comprises calcium carbonate, ascorbic acid, a glucosamine salt, vitamin D3, cross linked povidone, cross linked sodium carboxymethyl cellulose, microcrystalline cellulose, magnesium stearate and PEG 6000. After the weakly effervescent disintegrating formulation disintegrates in neutral pure water, e.g., 200 mL of neutral pure water, the pH of the suspension or solution formed is more than 4.9, such that the formulation is in the form of a dispersible tablet. The dissolution of the calcium in the weakly effervescent disintegration formulation may be more than 20% in 900 mL of neutral pure water under stirring at a speed of 75 RPM when measured at 30 min.

Another embodiment of the present invention relates to a weakly effervescent formulation which comprises calcium carbonate, ascorbic acid, glucosamine sulfate, vitamin D3 and chondroitin sulfate. In a preferred embodiment, the weakly effervescent formulation comprises calcium carbonate, ascorbic acid, glucosamine sulfate, vitamin D3, chondroitin sulfate, cross linked povidone, cross linked sodium carboxymethyl cellulose, microcrystalline cellulose, magnesium stearate and PEG 6000. After the weakly effervescent disintegrating formulation disintegrates in neutral pure water, e.g., 200 mL of neutral pure water, the pH of the suspension or solution formed is greater than 4.9, preferably from 4.9 to 7.0, such that the formulation is in the form of a dispersible tablet or tablet. The dissolution of the calcium in the weakly effervescent formulation may be more than 20% in 900 mL of neutral pure water under stirring at a speed of 75 RPM when measured at 30 min.

Another embodiment of the present invention relates to a weakly effervescent formulation which comprises calcium carbonate, ascorbic acid, and one or more vitamins selected from vitamin A, vitamin E, vitamin D3, vitamin K, vitamin B1, vitamin B2, vitamin B6, vitamin B12 and folic acid. In a preferred embodiment, the weakly effervescent formulation comprises calcium carbonate, ascorbic acid, cross linked povidone, cross linked sodium carboxymethyl cellulose, microcrystalline cellulose, magnesium stearate, PEG 6000, and one or more vitamins selected from vitamin A, vitamin E, vitamin D3, vitamin K, vitamin B11, vitamin B2, vitamin B6, vitamin B12 and folic acid. After the weakly effervescent disintegrating formulation disintegrates in neutral pure water, e.g., 200 mL of neutral pure water, the pH of the suspension or solution formed is greater than 4.9, preferably from 4.9 to 7.0, such that the formulation is in the form of a dispersible tablet. In addition, the dissolution of the calcium in the weakly effervescent formulation may be more than 20% in 900 mL of neutral pure water under stirring at a speed of 75 RPM when measured at 30 min.

Another embodiment of the present invention relates to a weakly effervescent formulation which comprises calcium carbonate, ascorbic acid, and one or more food supplements selected from vitamin A, vitamin D3, vitamin E, vitamin K, vitamin B1, vitamin B2, vitamin B6, vitamin B12, folic acid, nicotinic acid, pantothenic acid, choline, biotin, iron, copper, zinc, potassium, chloride, magnesium, sodium, and salts thereof, and mixtures thereof. In a preferred embodiment, the weakly effervescent formulation comprises calcium carbonate, ascorbic acid, cross linked povidone, cross linked sodium carboxymethyl cellulose, microcrystalline cellulose, magnesium stearate, PEG 6000 and one or more food supplements selected from vitamin A, vitamin D3, vitamin E, vitamin K, vitamin B1, vitamin B2, vitamin B6, vitamin B12, folic acid, nicotinic acid, pantothenic acid, choline, biotin, iron, copper, zinc, potassium, chloride, magnesium, sodium, or their salts, and mixtures thereof. After the weakly effervescent disintegrating formulation disintegrates in neutral pure water, e.g., 200 mL of neutral pure water, the pH of the suspension or solution formed is greater than 4.9, such that the formulation is in the form of a dispersible tablet. The dissolution of the calcium in the weakly effervescent formulation may be more than 20% in 900 mL of neutral pure water under stirring at a speed of 75 RPM when measured at 30 min.

Advantageously, the weakly effervescent tablet formulations according to the present invention are suitable for formulation with a wide range of APIs.

Another embodiment of the present invention relates to a weakly effervescent formulation which comprises a hydrogen carbonate salt (such as sodium hydrogen carbonate or potassium hydrogen carbonate), a weak organic acid, and an API selected from hemostatic drugs, antihypertensive drugs, 5-HT3 antagonist antiemetic drugs, sartans, hypoglycemic drugs, anti-allergic drugs, contraceptives, antidepressants, NSAIDs, COX-2 inhibitors and anti-epileptic drugs.

In a preferred embodiment, the weakly effervescent formulation comprises a hydrogen carbonate salt (such as sodium hydrogen carbonate or potassium hydrogen carbonate), a weak organic acid, an API selected from hemostatic drugs, antihypertensive drugs, 5-HT3 antagonist antiemetic drugs, sartans, hypoglycemic drugs, anti-allergic drugs, contraceptives, antidepressants, NSAIDs, COX-2 inhibitors and anti-epileptics, cross linked povidone, cross linked sodium carboxymethyl cellulose, microcrystalline cellulose, magnesium stearate and PEG 6000.

In another preferred embodiment, the weakly effervescent formulation comprises a carbonate salt, such as calcium carbonate and a weak acid selected from malic acid, maleic acid, succinic acid, adipic acid, fumaric acid, tartaric acid, ascorbic acid; an API; and one or more excipients such as filling agents, diluents, lubricants and expanders.

In another preferred embodiment, weakly effervescent formulation comprises sodium bicarbonate and a weak acid, such as sodium hydrogen tartrate or monosodium malate, an API and one or more excipients such as filling agents, diluents, lubricants, and expanders.

Advantageously, after the weakly effervescent disintegrating formulation disintegrates in neutral pure water, e.g., 200 mL of neutral pure water, the pH of the suspension or solution formed is greater than 4.9, such that the formulation is in the form of an ODT or a dispersible tablet. The weight of the tablet determines the time of disintegration, which in turn determines whether it is an ODT or a dispersible tablet.

The time of disintegration determines the classification of the formulation. Dispersible tablets typically disintegrate in water within 3 minutes and are generally administered in two ways: (1) dispersing the dosage form in a glass of aqueous liquid or solution (such as water, juice or the like), and then the resultant suspension or solution is taken by mouth, or (2) the dispersing dosage form is placed in the oral cavity without the need for drinking water or other fluid. Accordingly:
time of disintegration: less than 1 minute=ODT
less than 3 minutes, but longer than 1 minute=dispersible tablet.

The time of disintegration depends on the force of expanding in the tablet. The force may be a physical force arising, for example, from the absorption of water, as from cross linked povidone, and cross linked carboxymethyl cellulose sodium. In other embodiments the force may be due to a chemical reaction producing the explosion within the tablet due to CO₂ production. However, the chemical reaction should not be too vigorous otherwise it will burn the tongue. Accordingly, the present invention is based on weakly effervescent disintegrating tablets wherein the pH of the resultant solution or suspension after disintegration of dissolution is greater than 4.9. The pH above 4.9 inherently restricts the severity of the acid-base reaction. The higher the pH, such as from pH 6.0 to pH 7, the less severe the effervescent CO₂-producing reaction will be.

The weakly effervescent formulations according to the present invention are suitable for use with a wide range of APIs, including vitamins, minerals, food nutrients, triptan drugs, dihydropyridine calcium channel blockers, antihistamines, 5-HT3 antagonist antiemetic drugs, sartans, alpha-glucosidase inhibitors, antifibrinolytic drugs, non-classical antidepressants, selective COX-2 inhibitors, NSAIDs, hormone contraceptives and anti-epileptics.

In an embodiment, some formulations have a small amount of API per tablet (e.g., less than 25 mg API per tablet), so the final tablet weight may weigh less than 150 mg. In an embodiment, such formulations may be formulated into a dispersible tablet which disintegrates in water within about 3 minutes. In another embodiment, such formulations advantageously may be readily formulated as an ODT which disintegrates in water within about 1 minute. In a preferred embodiment, the amount of API may be less than 20 mg, less than 15 mg, less than 10 mg, less than 5 mg, or less than 3 mg per tablet. In another embodiment, the API is present in an amount less than 12 mg per tablet.

In an embodiment, some formulations have a moderate amount of API per tablet, for example, an amount of about 25-50 mg per tablet, or about 50-100 mg per tablet, or about 100-150 mg per tablet. In another embodiment, some formulations have a high dosage of API, e.g., about 150-200 mg API per tablet. Such formulations are more difficult to make into an ODT as the total tablet weight including the excipients will be more than 500 mg. In an embodiment, tablets having a moderate amount of API (e.g., 50 mg to 100 mg API) may be made into an ODT or a dispersible tablet, however tablets containing high amounts of API (e.g., 100 mg to 200 mg API) may be made into dispersible tablets, as additional time is required for disintegration.

In preferred embodiments, the ODT or dispersible tablet is a suitably stable form of ODT or a dispersible tablet. In an embodiment, the ODT or dispersible tablet may be shelf stable for at least 2 months, or at least 4 months, or at least 6 months, or at least 10 months, or at least 12 months, or at least 18 months, or at least 24 months.

The weakly effervescent formulations according to the present invention are suitable for use with a wide range of APIs including triptans, dihydropyridine calcium channel blockers, antihistamines, sartans, 5-HT3 antagonist antiemetic drugs, alpha-glucosidase inhibitors, antifibrinolytic drugs, non-classical antidepressants, selective COX-2 inhibitors, NSAIDs, hormone contraceptives and anti-epileptics.

In a preferred embodiment, the stability of a formulation comprising a basic API may be improved by using a bicarbonate salt as the weak base.

The triptan class drugs includes rizatriptan, sumatriptan, zolmitriptan, naratriptan, almotriptan, eletriptan and frovatriptan, all of which are called acute medications and are designed to treat a migraine attack or cluster headache after the attack begins. By stopping the migraine, acute medications help alleviate the symptoms of migraines such as pain, nausea and sensitivity to light and sound. Rapid dissolution of such drugs in a weakly effervescent formulation, e.g., orodispersible or orally disintegrating tablets would be highly advantageous. In accordance with the present invention, triptan drugs, including rizatriptan, sumatriptan, zolmitriptan, naratriptan, almotriptan, eletriptan and frovatriptan, may be made into a weakly effervescent disintegrating formulations (e.g., tablets or granules) comprising (or consisting essentially of) a pair of weak acid with carbonate, and one or more other excipients, such as filling agents, diluents, lubricants, and expanders. After the weakly effervescent disintegrating formulation disintegrates in neutral pure water, the pH of the suspension or solution formed is greater than 4.9.

Other embodiments of the present invention relate to a weakly effervescent formulation which comprises calcium carbonate, an acid selected from malic acid, maleic acid, succinic acid, adipic acid, fumaric acid, tartaric acid and ascorbic acid, expander, diluent, filler, lubricant, and a triptan drug selected from rizatriptan, sumatriptan, zolmitriptan, naratriptan, almotriptan, eletriptan and frovatriptan. After the weakly effervescent disintegrating formulation of triptan disintegrates in neutral pure water, the pH of the suspension formed is more than 4.9, such that the formulation is in the form of an ODT.

Another embodiment of the present invention relates to a weakly effervescent formulation comprising calcium carbonate (e.g., 10 mg), malic acid (e.g., 10 mg), rizatriptan (e.g., 5 mg), cross linked povidone, microcrystalline cellulose, magnesium stearate and PEG 6000. After the weakly effervescent disintegrating formulation disintegrates in 200 mL of neutral pure water, the pH of the suspension or solution formed is greater than 4.9, such that the formulation is in the form of an oral dispersible tablet.

Another embodiment of the present invention relates to a weakly effervescent formulation which comprises calcium carbonate (e.g., 10 mg), malic acid (e.g., 10 mg), sumatriptan (e.g., 25 mg to 100 mg), cross linked povidone, cross linked sodium carboxymethyl cellulose, microcrystalline cellulose, magnesium stearate and PEG 6000. After the weakly effervescent disintegrating formulation disintegrates in 200 mL of neutral pure water, the pH of the suspension or solution formed is more than 4.9, such that the formulation is in the form of an oral dispersible tablet.

Another embodiment of the present invention relates to a weakly effervescent formulation which comprises calcium carbonate (e.g., 10 mg), malic acid (e.g., 10 mg), zolmitriptan (e.g., 2.5 mg to 5 mg), cross linked povidone, microcrystalline cellulose, magnesium stearate and PEG 6000. After the weakly effervescent disintegrating formulation disintegrates in 200 mL of neutral pure water, the pH of the suspension or solution formed is greater than 4.9, such that the formulation is in the form of an oral dispersible tablet.

Another embodiment of the present invention relates to a weakly effervescent formulation which comprises calcium carbonate (e.g., 10 mg), malic acid (e.g., 10 mg), naratriptan (e.g., 2.5 mg or 5 mg), cross linked povidone, microcrystalline cellulose, magnesium stearate and PEG 6000. After the weakly effervescent disintegrating formulation disintegrates in 200 mL of neutral pure water, the pH of the suspension formed is greater than 4.9, such that the formulation is in the form of an oral dispersible tablet.

Another embodiment of the present invention relates to a weakly effervescent formulation which comprises calcium carbonate (e.g., 10 mg), malic acid (e.g., 10 mg), almotriptan (e.g., 6.25 mg to 12.5 mg), cross linked povidone, microcrystalline cellulose, magnesium stearate and PEG 6000. After the weakly effervescent disintegrating formulation disintegrates in 200 mL of neutral pure water, the pH of the suspension or solution formed is greater than 4.9, such that the formulation is in the form of an oral dispersible tablet.

Another embodiment of the present invention relates to a weakly effervescent formulation which comprises calcium carbonate (e.g., 10 mg), malic acid (e.g., 15 mg), eletriptan (e.g., 40 mg), cross linked povidone, cross linked sodium carboxymethyl cellulose, microcrystalline cellulose, magnesium stearate and PEG 6000. After the weakly effervescent disintegrating formulation disintegrates in 200 mL of neutral pure water, the pH of the suspension or solution formed is greater than 4.9, such that the formulation is in the form of an oral dispersible tablet.

Another embodiment of the present invention relates to a weakly effervescent formulation which comprises calcium carbonate (e.g., 10 mg), malic acid (e.g., 14 mg), frovatriptan (e.g., 2.5 mg), cross linked povidone, microcrystalline cellulose, magnesium stearate and PEG 6000. After the weakly effervescent disintegrating formulation disintegrates in 200 mL of neutral pure water, the pH of the suspension or solution formed is greater than 4.9, such that the formulation is in the form of an oral dispersible tablet.

Another embodiment of the present invention relates to a weakly effervescent formulation which comprises calcium carbonate, malic acid, cross linked povidone, cross linked sodium carboxymethyl cellulose, microcrystalline cellulose, magnesium stearate, PEG 6000 and tramadol hydrochloride. After the weakly effervescent formulation of tramadol hydrochloride disintegrates in neutral pure water, the pH of the suspension formed is more than 4.9, such that the formulation is in the form of an ODT.

Another composition of the invention comprises calcium carbonate, an organic acid selected from malic acid, maleic acid, succinic acid, tartaric acid, citric acid and ascorbic acid, cross linked povidone, magnesium stearate and tramadol hydrochloride. After the composition of tramadol hydrochloride disintegrates in neutral pure water, the pH of the suspension formed is more than 4.9, such that the composition is in the form of an ODT.

Another composition of the present invention comprises calcium carbonate, an organic acid selected from malic acid, maleic acid, succinic acid, tartaric acid, citric acid and ascorbic acid, cross linked povidone, magnesium stearate and sildenafil citrate. After the composition of sildenafil citrate disintegrates in neutral pure water, the pH of the suspension formed is more than 4.9, such that the composition is in the form of an ODT.

Another composition of the present invention comprises calcium carbonate, malic acid, cross linked povidone, microcrystalline cellulose, magnesium stearate and sildenafil citrate. After the composition of sildenafil citrate disintegrates in neutral pure water, the pH of the suspension formed is more than 4.9, such that the formulation is in the form of an ODT.

### 5-HT3 (5-Hydroxytryptamine-3) Receptor Antagonists

Antiemetic drugs of the 5-HT3 (5-hydroxytryptamine-3) receptor antagonist class include ondansetron, dolasetron, granisetron, palonosetron, and ramosetron. Such drugs are used to reduce or prevent vomiting, particularly cancer chemotherapy and radiotherapy-induced vomiting. 5-HT3 antagonists such as ondansetron HCl, dolasetron HCl, granisetron HCl, palonosetron HCl, and ramosetron HCl cannot be made into ODTs by the lyophilization method because the API is soluble in water. In accordance with the present invention, water soluble salts of 5-HT3 antagonists such as ondansetron HCl, dolasetron HCl, granisetron HCl, palonosetron HCl, and ramosetron HCl may be made into a weakly effervescent disintegrating formulation e.g., orodispersible or orally disintegrating tablets or granules comprising (or consisting essentially of) a pair of weak acid and carbonate, and one or more excipients selected from a filling agent, diluent, lubricant and expander. After the weakly effervescent disintegrating formulation disintegrates in the neutral pure water, the pH of the suspension or solution formed is greater than 4.9.

In another embodiment the present invention relates to a weakly effervescent formulation which comprises calcium carbonate (e.g., 10 mg), malic acid (e.g., 14 mg), a 5-HT3 (5-hydroxytryptamine-3) receptor antagonist selected from ondansetron HCl, dolasetron HCl, granisetron HCl, palonosetron HCl, and ramosetron HCl or a base thereof, cross linked povidone, cross linked sodium carboxymethyl cellulose, microcrystalline cellulose, magnesium stearate and PEG 6000. After the weakly effervescent disintegrating formulation disintegrates in neutral pure water, e.g., 200 mL neutral pure water, the pH of the suspension or solution formed is greater than 4.9, such that the formulation is in the form of an oral dispersible tablet.

Other embodiments of the present invention relate to weakly effervescent formulations comprising calcium carbonate, an acid selected from malic acid, maleic acid, succinic acid, adipic acid, fumaric acid, tartaric acid and ascorbic acid, expander, diluent, filler, lubricant and an antiemetic drug of 5-HT3 antagonist selected from ondansetron, dolasetron, granisetron, palonosetron and ramosetron, or a salt thereof. After the weakly effervescent disintegrating formulation of 5-HT3 antagonist drug disintegrates in neutral pure water, the pH of the suspension formed is more than 4.9, such that the formulation is in the form of an ODT.

Another embodiment of the present invention relates to a weakly effervescent formulation which comprises calcium carbonate, malic acid, cross linked povidone, cross linked sodium carboxymethyl cellulose, microcrystalline cellulose, magnesium stearate, PEG 6000 and ondansetron HCl. After the weakly effervescent formulation of ondansetron HCl disintegrates in neutral pure water, the pH of the suspension formed is more than 4.9, such that the formulation is in the form of an ODT.

Another embodiment of the present invention relates to a weakly effervescent formulation which comprises calcium carbonate, malic acid, cross linked povidone, cross linked sodium carboxymethyl cellulose, microcrystalline cellulose, magnesium stearate, PEG 6000 and granisetron HCl. After the weakly effervescent formulation of granisetron HCl disintegrates in the neutral water, the pH of the suspension formed is more than 4.9, such that the formulation is in the form of an ODT.

Another embodiment of the present invention relates to a weakly effervescent formulation which comprises calcium carbonate, malic acid, cross linked povidone, cross linked sodium carboxymethyl cellulose, microcrystalline cellulose, magnesium stearate, PEG 6000 and ramosetron HCl. After the weakly effervescent formulation of ramosetron HCl disintegrates in neutral pure water, the pH of the suspension formed is more than 4.9, such that the formulation is in the form of an ODT.

Another embodiment of the present invention relates to a weakly effervescent formulation which comprises calcium carbonate, malic acid, cross linked povidone, cross linked sodium carboxymethyl cellulose, microcrystalline cellulose, magnesium stearate, PEG 6000 and palonosetron. After the weakly effervescent formulation of palonosetron disintegrates in neutral pure water, the pH of the suspension formed is more than 4.9, such that the formulation is in the form of an ODT.

Another embodiment of the present invention relates to a weakly effervescent formulation which comprises calcium carbonate, malic acid, cross linked povidone, cross linked sodium carboxymethyl cellulose, microcrystalline cellulose, magnesium stearate, PEG 6000 and dolasetron HCl. After the weakly effervescent formulation of dolasetron HCl disintegrates in neutral pure water, the pH of the suspension formed is more than 4.9, such that the formulation is in the form of an ODT.

### Calcium Channel Blockers

Calcium channel blockers such as amlodipine, L-amlodipine, felodipine, nitrendipine, nimodipine, benidipine, nifedipine have a long-lasting antihypertensive effect, and may be taken by patients once or twice daily. However, when a patient experiences a rapid increase in blood pressure, it is preferable to take a fast acting antihypertensive drug. In accordance with the present invention, calcium channel blockers such as amlodipine, L-amlodipine, felodipine, nitrendipine, benidipine, nimodipine, nifedipine may be made into a weakly effervescent disintegrating formulation e.g., orodispersible or orally disintegrating tablets or granules comprising (or consisting essentially of) a weak acid and carbonate, and one or more excipients such as e.g., a filling agent, diluent, lubricant and expander. After the weakly effervescent disintegrating formulation disintegrates in the neutral (pure) water, the pH of the suspension or solution formed is greater than 4.9.

In another embodiment, the present invention relates to a weakly effervescent formulation which comprises a carbonate salt; a weak acid; a calcium channel blocker selected from amlodipine, L-amlodipine, felodipine, nitrendipine, benidipine, nifedipine, lacidipine, nimodipine, lecardipine, manidipine, nicardipine; cross linked povidone, cross linked sodium carboxymethyl cellulose, microcrystalline cellulose, magnesium stearate and PEG 6000. After the weakly effervescent disintegrating formulation disintegrates in neutral pure water, e.g., 200 mL of neutral pure water, the pH of the suspension or solution formed is greater than 4.9, such that the formulation is in the form of an oral dispersible tablet.

Other embodiments of the present invention relate to a weakly effervescent formulation which comprises sodium hydrogen carbonate, potassium hydrogen carbonate, a weak organic acid selected from monosodium malate, monosodium maleate, monosodium succinate, monosodium adipate, monosodium oxalate, monosodium citrate, monosodium tartrate, monopotassium malate, monopotassium maleate, monopotassium succinate, monopotassium adipate, monopotassium oxalate, monopotassium citrate, monopotassium tartrate and a dihydropyridine calcium channel blocker selected from nifedipine, felodipine, amlodipine, levamlodipine, benidipine, nitrendipine, nimodipine and lacidipine. After the weakly effervescent disintegrating formulation of dihydropyridine calcium channel blocker disintegrates in neutral pure water, the pH of the suspension or solution formed is from 4.9 to 7.5, such that the formulation is in the form of an ODT.

Another embodiment of the present invention relates to a weakly effervescent formulation which comprises sodium hydrogen carbonate, monosodium tartrate, cross linked povidone, microcrystalline cellulose, magnesium stearate, PEG 6000 and nifedipine. After the weakly effervescent formulation of nifedipine disintegrates in the neutral water, the pH of the suspension or solution formed is greater than 4.9, such that the formulation is in the form of an ODT.

Another composition of present invention comprises sodium hydrogen carbonate, a weak acid selected from monosodium tartrate, monosodium malate, monosodium maleate, monosodium succinate, monosodium citrate, cross linked povidone, magnesium stearate and nifedipine. After the composition of nifedipine disintegrates in the neutral water, the pH of the suspension formed is greater than 4.9, such that the formulation is in the form of an ODT.

Another embodiment of the present invention relates to a weakly effervescent formulation which comprises sodium hydrogen carbonate, monosodium tartrate, cross linked povidone, microcrystalline cellulose, magnesium stearate, PEG 6000 and felodipine. After the weakly effervescent formulation of felodipine disintegrates in neutral pure water, the pH of the suspension or solution formed is greater than 4.9, such that the formulation is in the form of an ODT.

Another embodiment of the present invention relates to a weakly effervescent formulation which comprises sodium hydrogen carbonate, monosodium tartrate, cross linked povidone, microcrystalline cellulose, magnesium stearate, PEG 6000 and amlodipine. After the weakly effervescent formulation of amlodipine disintegrates in neutral pure water, the pH of the suspension or solution formed is greater than 4.9, such that the formulation is in the form of an ODT.

Another embodiment of the present invention relates to a weakly effervescent formulation which comprises sodium hydrogen carbonate, monosodium tartrate, cross linked povidone, microcrystalline cellulose, magnesium stearate, PEG 6000 and levamlodipine. After the weakly effervescent formulation of levamlodipine disintegrates in neutral pure water, the pH of the suspension or solution formed is greater than 4.9, such that the formulation is in the form of an ODT.

Another embodiment of the present invention relates to a weakly effervescent formulation which comprises sodium hydrogen carbonate, monosodium tartrate, cross linked povidone, microcrystalline cellulose, magnesium stearate, PEG 6000 and lacidipine. After the weakly effervescent formulation of lacidipine disintegrates in neutral pure water, the pH of the suspension or solution formed is greater than 4.9, such that the formulation is in the form of an ODT.

Another embodiment of the present invention relates to a weakly effervescent formulation which comprises sodium hydrogen carbonate, monosodium tartrate, cross linked povidone, microcrystalline cellulose, magnesium stearate, PEG 6000 and benidipine. After the weakly effervescent formulation of benidipine disintegrates in the neutral water, the pH of the suspension or solution formed is more than 4.9, such that the formulation is in the form of an ODT.

Another embodiment of the present invention relates to a weakly effervescent formulation which comprises sodium hydrogen carbonate, monosodium tartrate, cross linked povidone, microcrystalline cellulose, magnesium stearate, PEG 6000 and nitrendipine. After the invention of nitrendipine disintegrates in the neutral water, the pH of the suspension formed is more than 4.9, such that the formulation is in the form of an ODT.

Another embodiment of the present invention relates to a weakly effervescent formulation which comprises sodium bicarbonate (e.g., 10 mg), sodium hydrogen tartrate (e.g., 13 mg), nimodipine (e.g., 10 mg), cross linked povidone, microcrystalline cellulose, magnesium stearate and PEG 6000. After the weakly effervescent disintegrating formulation disintegrates in 200 mL of neutral pure water, the pH of the suspension or solution formed is greater than 4.9, such that the formulation is in the form of an oral dispersible tablet.

Another composition of the present invention comprises sodium bicarbonate, a weak acid selected from the group consisting of monosodium tartrate, monosodium malate, monosodium maleate, monosodium succinate, monosodium citrate, nimodipine, cross linked povidone, and magnesium stearate. After the composition disintegrates in 200 mL of neutral pure water, the pH of the suspension formed is greater than 4.9, such that the formulation is in the form of an oral dispersible tablet.

### Antihistamines

Antihistamines are a class of APIs that block histamine release from histamine-1 receptors and are mostly used to treat allergies or cold and flu symptoms, though some first generation antihistamine may also be used for other conditions. Antihistamines are very useful for relieving symptoms of an allergic reaction, such as edema and inflammation. In accordance with the present invention, antihistamines such as levocetirizine, hydroxyzine, cetirizine, promethazine, fexofenadine, loratadine, terfenadine, desloratadine, chlorpheniramine, dexchlorpheniramine, clemastine, triprolidine and diphenhydramine may be made into a weakly effervescent disintegrating formulation, e.g., orodispersible or orally disintegrating tablets or granules, comprising (or consisting essentially of) a weak acid and carbonate, and one or more excipients selected from a filling agent, diluent, lubricant and expander. After the weakly effervescent disintegrating formulation disintegrates in the neutral pure water, the pH of the suspension or solution formed is greater than 4.9.

In another embodiment the present invention relates to a weakly effervescent formulation which comprises a carbonate salt, a weak acid, an antihistamine selected from levocetirizine, hydroxyzine, cetirizine, promethazine, fexofenadine, diphenhydramine, loratadine, terfenadine, desloratadine, chlorpheniramine, dexchlorpheniramine, clemastine, triprolidine, cross linked povidone, cross linked sodium carboxymethyl cellulose, microcrystalline cellulose, magnesium stearate and PEG 6000. After the weakly effervescent disintegrating formulation disintegrates in 200 mL of neutral pure water, the pH of the suspension or solution formed is greater than 4.9, such that the formulation is in the form of an oral dispersible tablet.

Another composition of the present invention comprises calcium carbonate, an organic acid selected from the group consisting of malic acid, maleic acid, tartaric acid, succinic acid, citric acid, ascorbic acid and mixtures thereof, cross linked povidone and fexofenadine. After the composition of the present invention disintegrates in 200 mL of neutral pure water, the pH of the suspension formed is more than 4.9, such that the formulation is in the form of an ODT.

Other embodiments of the present invention relate to a weakly effervescent formulation which comprises calcium carbonate, malic acid and an antihistamine selected from cetirizine, levocetirizine, hydroxyzine, promethazine, fexofenadine, loratadine, terfenadine, desloratadine, chlorpheniramine, dexchlorpheniramine, clemastine, triprolidine and diphenhydramine. After the weakly effervescent disintegrating formulation of antihistamine disintegrates in neutral pure water, the pH of the suspension formed is more than 4.9, such that the formulation is in the form of an ODT.

Another composition of the present invention comprises calcium carbonate, an organic acid selected from the group consisting of malic acid, maleic acid, tartaric acid, succinic acid, citric acid, ascorbic acid, an antihistamine selected from cetirizine, levocetirizine, hydroxyzine, promethazine, fexofenadine, loratadine, terfenadine, desloratadine, chlorpheniramine, dexchlorpheniramine, clemastine, triprolidine and diphenhydramine, cross linked povidone, and magnesium stearate. After the composition of the present invention disintegrates in 200 mL of neutral pure water, the pH of the suspension formed is more than 4.9, such that the formulation is in the form of an ODT.

Another embodiment of the present invention relates to a weakly effervescent formulation which comprises calcium carbonate, malic acid, cross linked povidone, cross linked sodium carboxymethyl cellulose, microcrystalline cellulose, magnesium stearate, PEG 6000 and cetirizine. After the weakly effervescent disintegrating formulation of cetirizine disintegrates in neutral pure water, the pH of the suspension formed is more than 4.9, such that the formulation is in the form of an ODT.

Another embodiment of the present invention relates to a weakly effervescent formulation which comprises calcium carbonate, malic acid, cross linked sodium carboxymethyl cellulose, microcrystalline cellulose, magnesium stearate, PEG 6000 and levocetirizine. After the weakly effervescent disintegrating formulation of levocetirizine disintegrates in neutral pure water, the pH of the suspension formed is more than 4.9, such that the formulation is in the form of an ODT.

Another embodiment of the present invention relates to a weakly effervescent formulation which comprises calcium carbonate, malic acid, cross linked povidone, cross linked sodium carboxymethyl cellulose, microcrystalline cellulose, magnesium stearate, PEG 6000 and loratadine. After the weakly effervescent disintegrating formulation of loratadine disintegrates in neutral pure water, the pH of the suspension formed is more than 4.9, such that the formulation is in the form of an ODT.

Another embodiment of the present invention relates to a weakly effervescent formulation which comprises calcium carbonate, malic acid, cross linked povidone, cross linked sodium carboxymethyl cellulose, microcrystalline cellulose, magnesium stearate, PEG 6000 and desloratadine. After the weakly effervescent disintegrating formulation of desloratadine disintegrates in neutral pure water, the pH of the suspension formed is more than 4.9, such that the formulation is in the form of an ODT.

Another embodiment of the present invention relates to a weakly effervescent formulation which comprises calcium carbonate, malic acid, cross linked povidone, cross linked sodium carboxymethyl cellulose, microcrystalline cellulose, magnesium stearate, PEG 6000 and fexofenadine. After the weakly effervescent formulation of fexofenadine disintegrates in neutral pure water, the pH of the suspension formed is more than 4.9, such that the formulation is in the form of an ODT.

Another embodiment of the present invention relates to a weakly effervescent formulation which comprises calcium carbonate, malic acid, cross linked povidone, cross linked sodium carboxymethyl cellulose, microcrystalline cellulose, magnesium stearate, PEG 6000 and terfenadine. After the weakly effervescent formulation of terfenadine disintegrates in neutral pure water, the pH of the suspension formed is more than 4.9, such that the formulation is in the form of an ODT

Another embodiment of the present invention relates to a weakly effervescent formulation which comprises calcium carbonate, malic acid, cross linked povidone, cross linked sodium carboxymethyl cellulose, microcrystalline cellulose, magnesium stearate, PEG 6000 and chlorpheniramine. After the weakly effervescent formulation of chlorpheniramine disintegrates in neutral pure water, the pH of the suspension formed is more than 4.9, such that the formulation is in the form of an ODT.

Another embodiment of the present invention relates to a weakly effervescent formulation which comprises calcium carbonate, malic acid, cross linked povidone, cross linked sodium carboxymethyl cellulose, microcrystalline cellulose, magnesium stearate, PEG 6000 and clemastine. After the weakly effervescent formulation of clemastine disintegrates in neutral pure water, the pH of the suspension formed is more than 4.9, such that the formulation is in the form of an ODT.

Another composition of the present invention comprises calcium carbonate, fexofenadine, cross linked povidone, an organic acid selected from malic acid, maleic acid, tartaric acid, succinic acid, ascorbic acid, citric acid and magnesium stearate. After the composition of the fexofenadine disintegrates in neutral pure water, the pH of the suspension formed is more than 4.9, such that the composition is an ODT.

### Alpha-Glucosidase Inhibitors

Alpha-glucosidase inhibitors such as miglitol (miglibose), acarbose and voglibose work by competitive and reversible inhibition of intestinal enzymes. They slow the digestion of carbohydrates and delay glucose absorption. This results in a smaller and slower rise in blood glucose levels following meals, and effectively throughout the day. In accordance with the present invention, miglitol (miglibose), acarbose and voglibose may be made into a weakly effervescent disintegrating formulation e.g., orodispersible or orally disintegrating tablet or granules comprising (or consisting essentially of) a pair of weak acid and a carbonate, and one or more excipients selected from a filling agent, a diluent, a lubricant and an expander. After the weakly effervescent disintegrating formulation of alpha-glucosidase inhibitors disintegrates in the neutral pure water, the pH of the suspension or solution formed is greater than 4.9.

Other embodiments of the present invention relate to a weakly effervescent formulation which comprises calcium carbonate, a binary acid selected from malic acid, maleic acid, adipic acid, fumaric acid, tartaric acid, succinic acid, ascorbic acid and mixtures thereof; expander, diluent, filler, lubricant and an alpha-glucosidase inhibitors selected from miglitol (miglibose), acarbose and voglibose. After the weakly effervescent disintegrating formulation of alpha-glucosidase disintegrates in neutral pure water, the pH of the suspension formed is more than 4.9, such that the formulation is in the form of an ODT or a dispersible tablet.

In another embodiment the present invention relates to a weakly effervescent formulation which comprises a carbonate salt, a weak acid, an alpha-glucosidase inhibitor selected from miglitol (miglibose), acarbose and voglibose, cross linked povidone, cross linked sodium carboxymethyl cellulose, microcrystalline cellulose, magnesium stearate and PEG 6000. After the weakly effervescent disintegrating formulation disintegrates in 200 mL of neutral pure water, the pH of the suspension or solution formed is greater than 4.9, such that the formulation is in the form of an oral dispersible tablet.

Another embodiment of the present invention relates to a weakly effervescent formulation which comprises calcium carbonate, malic acid, cross linked povidone, cross linked sodium carboxymethyl cellulose, microcrystalline cellulose, magnesium stearate, PEG 6000 and acarbose. After the weakly effervescent formulation of acarbose disintegrates in neutral pure water, the pH of the suspension formed is more than 4.9, such that the formulation is in the form of an ODT.

Another embodiment of the weakly effervescent formulation comprises sodium hydrogen carbonate, monosodium citrate, cross linked povidone, cross linked sodium carboxymethyl cellulose, microcrystalline cellulose, magnesium stearate, PEG 6000 and voglibose. After the weakly effervescent formulation of voglibose disintegrates in neutral pure water, the pH of the suspension formed is more than 4.9, such that the formulation is in the form of an ODT.

Another embodiment of the present invention relates to a weakly effervescent formulation which comprises sodium hydrogen carbonate, monosodium citrate, cross linked povidone, cross linked sodium carboxymethyl cellulose, microcrystalline cellulose, magnesium stearate, PEG 6000 and miglibose. After the weakly effervescent formulation of miglitol (miglibose) disintegrates in neutral pure water, the pH of the suspension formed is more than 4.9, such that the formulation is in the form of an ODT or a dispersible tablet.

Another composition of the present invention comprises calcium carbonate, an organic acid selected from malic acid, maleic acid, tartaric acid, succinic acid, ascorbic acid and citric acid, cross linked povidone, microcrystalline cellulose, magnesium stearate and miglitol. After the composition of the miglitol disintegrates in neutral pure water, the pH of the suspension formed is more than 4.9, such that the formulation is in the form of an ODT.

Another composition of the present invention comprises calcium carbonate, acarbose, an organic acid selected from malic acid, maleic acid, tartaric acid, succinic acid, ascorbic acid and citric acid, cross linked povidone, microcrystalline cellulose and magnesium stearate. After the composition of the acarbose disintegrates in neutral pure water, the pH of the suspension formed is more than 4.9, such that the formulation is in the form of an ODT.

Another composition of the present invention comprises calcium carbonate, citric acid, acarbose, cross linked povidone, microcrystalline cellulose and magnesium stearate. After the composition of the acarbose disintegrates in neutral pure water, the pH of the suspension formed is more than 4.9, such that the formulation is in the form of an ODT.

### Angiotensin II Receptor Blockers (Sartans)

Sartans are angiotensin II receptor blockers, which is a class of pharmaceuticals that bind to and inhibit the angiotensin II receptor type 1 (AT1) and thereby block the arteriolar contraction and sodium retention effects of the renin-angiotensin system. Their main uses are in the treatment of hypertension, diabetic nephropathy and congestive heart failure. In accordance with the present invention, sartans such as losartan, candesartan, telmisartan, valsartan, fimasartan and irbesartan may be made into a weakly effervescent disintegrating formulation e.g., orodispersible or orally disintegrating tablet or granules comprising (or consisting essentially of) a pair of weak acid and a carbonate, and one or more excipients selected from a filling agent, a diluent, a lubricant and an expander. After the weakly effervescent disintegrating formulation of sartan disintegrates in the neutral pure water, the pH of the suspension or solution formed is greater than 4.9.

Another embodiment of the present invention relates to a weakly effervescent formulation which comprises sodium hydrogen carbonate, potassium hydrogen carbonate, monosodium malate, monosodium maleate, monosodium succinate, monosodium adipate, monosodium citrate, ascorbic acid, monosodium tartrate, monopotassium malate, monopotassium maleate, monopotassium succinate, monopotassium adipate, monopotassium oxalate, monopotassium citrate, monopotassium tartrate, and a sartan API selected from losartan, candesartan, telmisartan, valsartan, olmesartan, fimasartan and irbesartan or a salt thereof. After the weakly effervescent disintegrating formulation of sartan disintegrates in neutral pure water, the pH of the suspension or solution formed is more than 4.9, such that the formulation is in the form of an ODT or a dispersible tablet.

In another embodiment, the invention relates to a weakly effervescent formulation which comprises a carbonate salt, a weak acid (preferably not a binary acid), an angiotensin II receptor blocker (sartan) selected from the group consisting of losartan, candesartan, telmisartan, valsartan, olmesartan, fimasartan and irbesartan, cross linked povidone, cross linked sodium carboxymethyl cellulose, microcrystalline cellulose, magnesium stearate and PEG 6000. After the weakly effervescent disintegrating formulation disintegrates in 200 mL of neutral pure water, the pH of the suspension or solution formed is greater than 4.9, such that the formulation is in the form of an oral dispersible tablet.

Another embodiment of the present invention relates to a weakly effervescent formulation which comprises sodium hydrogen carbonate, monosodium tartrate, filling agent, diluent, lubricant, expander and an effective therapeutic dosage of losartan. After the weakly effervescent disintegrating formulation of losartan disintegrates in 200 mL of neutral pure water, the pH of the suspension or solution formed is greater than 4.9, such that the formulation is in the form of an ODT.

Another embodiment of the present invention relates to a weakly effervescent formulation which comprises sodium hydrogen carbonate, monosodium tartrate, filling agent, diluent, lubricant, expander and an effective therapeutic dosage of candesartan. After the weakly effervescent disintegrating formulation of candesartan disintegrates in 200 mL of neutral pure water, the pH of the suspension or solution formed is greater than 4.9, such that the formulation is in the form of an ODT.

Another embodiment of the present invention relates to a weakly effervescent formulation which comprises sodium hydrogen carbonate, monosodium tartrate, filling agent, diluent, lubricant, expander and an effective therapeutic dosage of telmisartan. After the weakly effervescent disintegrating formulation of telmisartan disintegrates in 200 mL of neutral pure water, the pH of the suspension or solution formed is greater than 4.9, such that the formulation is in the form of an ODT or a dispersible tablet.

Another embodiment of the present invention relates to a weakly effervescent formulation which comprises sodium hydrogen carbonate, monosodium tartrate, filling agent, diluent, lubricant, expander and an effective therapeutic dosage of valsartan. After the weakly effervescent disintegrating formulation of valsartan disintegrates in 200 mL of neutral pure water, the pH of the suspension or solution formed is greater than 4.9, such that the formulation is in the form of an ODT or a dispersible tablet.

Another embodiment of the present invention relates to a weakly effervescent formulation which comprises sodium hydrogen carbonate, monosodium tartrate, filling agent, diluent, lubricant, expander and an effective therapeutic dosage of fimasartan. After the weakly effervescent disintegrating formulation of fimasartan disintegrates in 200 mL of neutral pure water, the pH of the suspension or solution formed is greater than 4.9, such that the formulation is in the form of an ODT or a dispersible tablet.

Another embodiment of the present invention relates to a weakly effervescent formulation which comprises sodium hydrogen carbonate, monosodium tartrate, filling agent, diluent, lubricant, expander and an effective therapeutic dosage of irbesartan. After the weakly effervescent formulation of irbesartan disintegrates in 200 mL of neutral pure water, the pH of the suspension or solution formed is greater than 4.9, such that the formulation is in the form of an ODT or a dispersible tablet.

Another embodiment of the present invention relates to a weakly effervescent formulation which comprises sodium hydrogen carbonate, monosodium tartrate, microcrystalline cellulose, cross linked sodium carboxymethyl cellulose, magnesium stearate, PEG 6000 and olmesartan. After the weakly effervescent formulation of olmesartan disintegrates in neutral pure water, the pH of the suspension or solution formed is greater than 4.9, such that the formulation is in the form of an ODT.

Another composition of the present invention comprises sodium hydrogen carbonate, monosodium tartrate, cross linked sodium carboxymethyl cellulose, magnesium stearate and irbesartan. After the composition of irbesartan disintegrates in neutral pure water, the pH of the suspension formed is greater than 4.9, such that the formulation is in the form of an ODT or a dispersible tablet.

Another composition of the present invention comprises sodium hydrogen carbonate, a weak acid selected from the group consisting of monosodium tartrate, monosodium malate, monosodium maleate, monosodium succinate, monosodium citrate, monopotassium malate, monopotassium maleate, monopotassium succinate, monopotassium tartrate, cross linked sodium carboxymethyl cellulose, magnesium stearate and irbesartan. After the composition of irbesartan disintegrates in neutral pure water, the pH of the suspension formed is greater than 4.9, such that the formulation is in the form of an ODT or a dispersible tablet.

### Antifibrinolytic Drugs

Antifibrinolytic drugs comprise 6-aminocaproic acid, p-hydroxybenzylamine and tranexamic acid. Such drugs may inhibit the activation of fibrinogen, so that the former cannot be activated and become fibrinolytic enzyme, thus prolonging the dissolution period of blood clots. 6-aminocaproic acid, tranexamic acid and p-hydroxybenzylamine may be made into a weakly effervescent disintegrating formulation e.g., an orodispersible or orally disintegrating tablet or granules comprising (or consisting essentially of) a pair of weak acid and a carbonate, a filling agent, a diluent, a lubricant and an expander. After the weakly effervescent disintegrating formulation of antifibrinolytic drug disintegrates in the neutral water, the pH of the suspension formed is greater than 4.9. The formulation may be prepared in the form of an ODT or a dispersible tablet.

In an embodiment, the present invention relates to a weakly effervescent formulation which comprises a carbonate salt, a weak acid, an antifibrinolytic drug selected from 6-aminocaproic acid, tranexamic acid and p-hydroxybenzylamine, cross linked povidone, cross linked sodium carboxymethyl cellulose, microcrystalline cellulose, magnesium stearate and PEG 6000. After the weakly effervescent disintegrating formulation disintegrates in 200 mL of neutral pure water, the pH of the suspension or solution formed is more than 4.9, such that the formulation is in the form of a dispersible tablet.

Other embodiments of the present invention relate to weakly effervescent formulations comprising sodium bicarbonate, potassium bicarbonate; an acid selected from monosodium malate, monosodium maleate, monosodium succinate, monosodium adipate, monosodium oxalate, monosodium citrate, monosodium tartrate, monopotassium malate, monopotassium maleate, monopotassium succinate, monopotassium adipate, monopotassium oxalate, monopotassium tartrate and mixtures thereof; and an antifibrinolytic drug selected from 6-aminocaproic acid, tranexamic acid and p-hydroxybenzylamine. After the weakly effervescent disintegrating formulation of antifibrinolytic drug disintegrates in neutral pure water, the pH of the suspension formed is more than 4.9, such that the formulation is in the form of an ODT or a dispersible tablet.

Another embodiment of the present invention relates to a weakly effervescent formulation which comprises sodium bicarbonate, monosodium malate, cross linked sodium carboxymethyl cellulose, cross linked povidone, microcrystalline cellulose, magnesium stearate, PEG 6000 and 6-aminocaproic acid. After the weakly effervescent formulation of 6-aminocaproic acid disintegrates in neutral pure water, the pH of the suspension formed is more than 4.9, such that the formulation is in the form of an ODT or a dispersible tablet.

Another embodiment of the present invention relates to a weakly effervescent formulation which comprises sodium bicarbonate, monosodium malate, cross linked sodium carboxymethyl cellulose, cross linked povidone, microcrystalline cellulose, magnesium stearate, PEG 6000 and tranexamic acid. After the weakly effervescent formulation of tranexamic acid disintegrates in neutral pure water, the pH of the suspension formed is more than 4.9, such that the formulation is in the form of an ODT or a dispersible tablet.

Another embodiment of the present invention relates to a weakly effervescent formulation which comprises sodium bicarbonate, monosodium tartrate, cross linked povidone, cross linked sodium carboxymethyl cellulose, microcrystalline cellulose, magnesium stearate, PEG 6000 and p-hydroxybenzylamine. After the weakly effervescent formulation of p-hydroxybenzylamine disintegrates in neutral pure water, the pH of the suspension formed is more than 4.9, such that the formulation is in the form of an ODT or a dispersible tablet.

Another composition of the present invention comprises tranexamic acid, sodium bicarbonate, potassium bicarbonate, one acid selected from monosodium malate, monosodium maleate, monosodium succinate, monosodium citrate, monosodium tartrate, monopotassium malate, monopotassium maleate, monopotassium succinate, monopotassium citrate, monopotassium tartrate, cross linked sodium carboxymethyl cellulose, and magnesium stearate. After the composition of tranexamic acid disintegrates in neutral pure water, the pH of the suspension formed is more than 4.9, such that the formulation is in the form of an ODT or a dispersible tablet.

### Serotonin Reuptake Inhibitors

Serotonin reuptake inhibitors are a class of antidepressant, which act by inhibiting the reuptake of serotonin. The class of serotonin reuptake inhibitors comprise fluoxetine, paroxetine, sertraline, fluvoxamine, citalopram and escitalopram, which may be made into a weakly effervescent disintegrating formulation e.g., orodispersible or orally disintegrating tablets comprising (or consisting essentially of) a pair of weak acid and a carbonate, and one or more excipients selected from a filling agent, a diluent, a lubricant and an expander. After the weakly effervescent disintegrating formulation of serotonin reuptake inhibitors drugs disintegrates in the neutral water, the pH of the suspension or solution formed is greater than 4.9.

Other embodiments of the present invention relate to a weakly effervescent formulations comprising calcium carbonate, an acid selected from malic acid, maleic acid, succinic acid, adipic acid, fumaric acid, tartaric acid, citric acid and ascorbic acid and a serotonin reuptake inhibitor selected from fluoxetine, paroxetine, sertraline, fluvoxamine, citalopram and escitalopram. After the weakly effervescent disintegrating formulation of selective serotonin reuptake inhibitor disintegrates in neutral pure water, the pH of the suspension formed is more than 4.9, such that the formulation is in the form of an ODT or a dispersible tablet.

In another embodiment, the invention relates to a weakly effervescent formulation comprising calcium carbonate, malic acid, a serotonin reuptake inhibitor selected from fluoxetine, paroxetine, sertraline, fluvoxamine, citalopram and escitalopram or a salt thereof, cross linked povidone, cross linked sodium carboxymethyl cellulose, microcrystalline cellulose, magnesium stearate and PEG 6000. After the weakly effervescent disintegrating formulation disintegrates in 200 mL of neutral pure water, the pH of the suspension or solution formed is greater than 4.9, such that the formulation is in the form of an oral dispersible tablet.

Another embodiment of the present invention relates to a weakly effervescent formulation which comprises calcium carbonate, malic acid, cross linked povidone, cross linked sodium carboxymethyl cellulose, microcrystalline cellulose, magnesium stearate, PEG 6000 and fluoxetine. After the weakly effervescent formulation of fluoxetine disintegrates in neutral pure water, the pH of the suspension formed is more than 4.9, such that the formulation is in the form of an ODT.

Another embodiment of the present invention relates to a weakly effervescent formulation which comprises calcium carbonate, malic acid, cross linked povidone, cross linked sodium carboxymethyl cellulose, microcrystalline cellulose, magnesium stearate, PEG 6000 and paroxetine. After the weakly effervescent formulation of paroxetine disintegrates in neutral pure water, the pH of the suspension formed is more than 4.9, such that the formulation is in the form of an ODT.

Another embodiment of the present invention relates to a weakly effervescent formulation which comprises calcium carbonate, malic acid, cross linked povidone, cross linked sodium carboxymethyl cellulose, microcrystalline cellulose, magnesium stearate, PEG 6000 and sertraline. After the weakly effervescent formulation of sertraline disintegrates in neutral pure water, the pH of the suspension formed is more than 4.9, such that the formulation is in the form of an ODT or a dispersible tablet.

Another embodiment of the present invention relates to a weakly effervescent formulation which comprises calcium carbonate, malic acid, cross linked povidone, cross linked sodium carboxymethyl cellulose, microcrystalline cellulose, magnesium stearate, PEG 6000 and fluvoxamine. After the weakly effervescent formulation of fluvoxamine disintegrates in neutral pure water, the pH of the suspension formed is more than 4.9, such that the formulation is in the form of an ODT or a dispersible tablet.

Another embodiment of the present invention relates to a weakly effervescent formulation which comprises calcium carbonate, malic acid, cross linked povidone, cross linked sodium carboxymethyl cellulose, microcrystalline cellulose, magnesium stearate, PEG 6000 and citalopram. After the weakly effervescent formulation of citalopram disintegrates in neutral pure water, the pH of the suspension formed is more than 4.9, such that the formulation is in the form of an ODT or a dispersible tablet.

Another embodiment of the present invention relates to a weakly effervescent formulation which comprises calcium carbonate, malic acid, cross linked povidone, cross linked sodium carboxymethyl cellulose, microcrystalline cellulose, magnesium stearate, PEG 6000 and escitalopram. After the weakly effervescent formulation of escitalopram disintegrates in neutral pure water, the pH of the suspension formed is more than 4.9, such that the formulation is in the form of an ODT.

### Non-Classical Antidepressants

Non-classical antidepressants, including aripiprazole, olanzapine, quetiapine, ziprasidone, clozapine and paliperidone, may be made into a weakly effervescent disintegrating formulation e.g., orodispersible or orally disintegrating tablets or granules comprising (or consisting essentially of) a pair of weak acid and a carbonate, and one or more excipients selected from a filling agent, a diluent, a lubricant and an expander. After the weakly effervescent disintegrating formulation of the non-classical antidepressant drugs disintegrates in the neutral pure water, the pH of the suspension or solution formed is greater than 4.9.

Other embodiments of the present invention relate to weakly effervescent formulations comprising calcium carbonate, an acid selected from malic acid, maleic acid, succinic acid, adipic acid, fumaric acid, tartaric acid, citric acid and ascorbic acid and a non-classical antidepressant selected from aripiprazole, olanzapine, quetiapine, ziprasidone, clozapine and paliperidone. After the weakly effervescent disintegrating formulation of non-classical antidepressant disintegrates in neutral pure water, the pH of the suspension formed is more than 4.9, such that the formulation is in the form of an ODT or a dispersible tablet.

Another embodiment of the present invention relates to a weakly effervescent formulation which comprises calcium carbonate, tartaric acid, cross linked povidone, cross linked sodium carboxymethyl cellulose, microcrystalline cellulose, magnesium stearate, PEG 6000 and aripiprazole. After the weakly effervescent formulation of aripiprazole disintegrates in neutral pure water, the pH of the suspension formed is more than 4.9, such that the formulation is in the form of an ODT.

Another embodiment of the present invention relates to a weakly effervescent formulation which comprises calcium carbonate, tartaric acid, cross linked povidone, cross linked sodium carboxymethyl cellulose, microcrystalline cellulose, magnesium stearate, PEG 6000 and olanzapine. After the weakly effervescent formulation of olanzapine disintegrates in neutral pure water, the pH of the suspension formed is more than 4.9, such that the formulation is in the form of an ODT.

Another embodiment of the present invention relates to a weakly effervescent formulation which comprises calcium carbonate, tartaric acid, cross linked povidone, cross linked sodium carboxymethyl cellulose, microcrystalline cellulose, magnesium stearate, PEG 6000 and clozapine. After the weakly effervescent formulation of clozapine disintegrates in neutral pure water, the pH of the suspension formed is more than 4.9, such that the formulation is in the form of an ODT.

Another embodiment of the present invention relates to a weakly effervescent formulation which comprises calcium carbonate, tartaric acid, cross linked povidone, cross linked sodium carboxymethyl cellulose, microcrystalline cellulose, magnesium stearate, PEG 6000 and quetiapine. After the weakly effervescent formulation of quetiapine disintegrates in neutral pure water, the pH of the suspension formed is more than 4.9, such that the formulation is in the form of an ODT or a dispersible tablet.

Another embodiment of the present invention relates to a weakly effervescent formulation which comprises calcium carbonate, tartaric acid, cross linked povidone, cross linked sodium carboxymethyl cellulose, microcrystalline cellulose, magnesium stearate, PEG 6000 and ziprasidone HCl. After the weakly effervescent formulation of ziprasidone disintegrates in neutral pure water, the pH of the suspension formed is more than 4.9, such that the formulation is in the form of an ODT or a dispersible tablet.

Another embodiment of the present invention relates to a weakly effervescent formulation which comprises calcium carbonate, tartaric acid, cross linked povidone, cross linked sodium carboxymethyl cellulose, microcrystalline cellulose, magnesium stearate, PEG 6000 and paliperidone. After the weakly effervescent formulation of paliperidone disintegrates in neutral pure water, the pH of the suspension formed is more than 4.9, such that the formulation is in the form of an ODT.

Another embodiment of the present invention relates to a weakly effervescent formulation which comprises calcium carbonate, tartaric acid, cross linked povidone, cross linked sodium carboxymethyl cellulose, microcrystalline cellulose, magnesium stearate, PEG 6000 and nicorandil. After the weakly effervescent formulation of nicorandil disintegrates in neutral pure water, the pH of the suspension formed is more than 4.9, such that the formulation is in the form of an ODT.

### Non-Steroidal Anti-Inflammatory Drugs

Non-steroidal anti-inflammatory drugs are used to treat osteoarthritis, rheumatoid arthritis, acute pain, dysmenorrhea and menstrual symptoms.

Other embodiments of the present invention relate to a weakly effervescent formulation which comprises calcium carbonate, one acid selected from malic acid, maleic acid, adipic acid, fumaric acid, tartaric acid, succinic acid and ascorbic acid, expander, diluent, filler, lubricant and a nonsteroidal anti-inflammatory drug selected from diclofenac, ibuprofen, indomethacin, ketoprofen, naproxen, oxaprozin and piroxicam. After the weakly effervescent disintegrating formulation of the nonsteroidal anti-inflammatory drug disintegrates in neutral pure water, the pH of the suspension formed is more than 4.9, such that the formulation is in the form of an ODT or a dispersible tablet.

Another embodiment of the present invention relates to a weakly effervescent formulation which comprises calcium carbonate, malic acid, cross linked povidone, cross linked sodium carboxymethyl cellulose, microcrystalline cellulose, magnesium stearate, PEG 6000 and ibuprofen. After the weakly effervescent formulation of ibuprofen disintegrates in the neutral water, the pH of the suspension formed is more than 4.9, such that the formulation is in the form of an ODT or a dispersible tablet.

Another composition of the present invention comprises ibuprofen, calcium carbonate, one acid selected from malic acid, tartaric acid, succinic acid, maleic acid, citric acid, ascorbic acid, cross linked povidone, microcrystalline cellulose and magnesium stearate. After the composition of the ibuprofen disintegrates in the neutral water, the pH of the suspension formed is more than 4.9, such that the formulation is in the form of an ODT or a dispersible tablet.

Another embodiment of the present invention relates to a weakly effervescent formulation which comprises calcium carbonate, malic acid, cross linked povidone, cross linked sodium carboxymethyl cellulose, microcrystalline cellulose, magnesium stearate, PEG 6000 and piroxicam. After the weakly effervescent formulation of piroxicam disintegrates in neutral pure water, the pH of the suspension formed is more than 4.9, such that the formulation is in the form of an ODT.

Another embodiment of the present invention relates to a weakly effervescent formulation which comprises calcium carbonate, malic acid, cross linked povidone, cross linked sodium carboxymethyl cellulose, microcrystalline cellulose, magnesium stearate, PEG 6000 and oxaprozin. After the weakly effervescent formulation of oxaprozin disintegrates in the neutral water, the pH of the suspension formed is more than 4.9, such that the formulation is in the form of an ODT.

Another embodiment of the present invention relates to a weakly effervescent formulation which comprises sodium hydrogen carbonate, monosodium tartrate, cross linked povidone, cross linked sodium carboxymethyl cellulose, microcrystalline cellulose, magnesium stearate, PEG 6000 and sodium valproate. After the weakly effervescent formulation disintegrates in neutral pure water, the pH of the suspension formed is more than 4.9, such that the formulation is in the form of an ODT or a dispersible tablet.

Another embodiment of the present invention relates to a weakly effervescent formulation which comprises sodium hydrogen carbonate, monosodium tartrate, cross linked povidone, cross linked sodium carboxymethyl cellulose, microcrystalline cellulose, magnesium stearate, PEG 6000 and phenytoin sodium. After the weakly effervescent formulation disintegrates in neutral pure water, the pH of the suspension formed is more than 4.9, such that the formulation is in the form of an ODT or a dispersible tablet.

Another composition of the present invention comprises phenytoin sodium, sodium bicarbonate, potassium bicarbonate, one acid selected from monosodium malate, monosodium maleate, monosodium succinate, monosodium citrate, monosodium tartrate, monopotassium malate, monopotassium maleate, monopotassium succinate, monopotassium citrate, monopotassium tartrate, cross linked povidone, cross linked sodium carboxymethyl cellulose, and magnesium stearate. After the composition of phenytoin sodium disintegrates in neutral pure water, the pH of the suspension formed is more than 4.9, such that the formulation is in the form of an ODT or a dispersible tablet.

### Selective COX-2 Inhibitors

The composition of celecoxib comprises 65 mg to 200 mg celecoxib, 65 mg to 800 mg of an organic acid which is selected from ascorbic acid, malic acid, maleic acid, succinic acid, tartaric acid and citric acid or a mixture thereof, 50 mg to 160 mg cross linked povidone, 50 mg to 160 mg cross linked carboxymethyl sodium cellulose, magnesium stearate and a small amount of PVPK30; wherein the formulation is in the form of a granule, capsule, tablet, which has a dissolution rate of more than 60% of celecoxib in a 1000 mL of pH 7 sodium phosphate buffer solution containing 2.5 g of sodium dodecyl sulfate under 50 RPM at 120 minutes after dissolution test.

Another composition of celecoxib comprises 65 mg to 200 mg of celecoxib, 65 mg to 800 mg of an organic acid selected from ascorbic acid, malic acid, maleic acid, succinic acid, tartaric acid and citric acid, 30 mg to 60 mg calcium carbonate, 65 mg to 160 mg cross linked povidone, 65 mg to 160 mg cross linked carboxymethyl sodium cellulose, magnesium stearate and a small amount of PVPK30; wherein the formulation is in the form of a granule or a tablet which has a dissolution rate of more than 60% of celecoxib in a 1000 mL of pH 7 sodium phosphate buffer solution containing 2.5 g of sodium dodecyl sulfate under 50 RPM at 120 minutes after dissolution test and a dissolution of more than 85% of calcium ions in 900 mL of neutral water.

Another composition of imrecoxib comprises 40 mg to 100 mg of imrecoxib, 40 mg to 480 mg of an organic acid selected from ascorbic acid, malic acid, maleic acid, succinic acid, tartaric acid and citric acid, wherein the formulation is in the form of granules, a capsule or a tablet.

Another composition of imrecoxib comprises 40 mg to 100 mg imrecoxib, 40 mg to 480 mg of an organic acid selected from ascorbic acid, malic acid, maleic acid, succinic acid, tartaric acid and citric acid, 30 mg to 60 mg calcium carbonate; wherein the formulation is in the form of granules, a tablet or a capsule.

Another composition of the invention comprises 65 mg to 160 mg celecoxib, 65 mg to 800 mg malic acid, 50 mg to 160 mg cross linked povidone, 50 mg to 160 mg cross linked carboxymethyl sodium cellulose, magnesium stearate and a small amount of PVPK30; wherein the formulation is in the form of a granule, a capsule, or a tablet, which has a dissolution rate of more than 60% of celecoxib in a 1000 mL of 0.25% sodium dodecyl sulfate/pH 7 Sodium Phosphate Buffer solution under 50 RPM at 120 minutes after dissolution test.

Another composition of the invention comprises 65 mg to 160 mg celecoxib, 65 mg to 800 mg of maleic acid, 50 mg to 160 mg of cross linked povidone, 50 mg to 160 mg of cross linked carboxymethyl sodium cellulose, magnesium stearate and a small amount of PVPK30; wherein the formulation is in the form of granules, a capsule or a tablet, which has a dissolution rate of more than 60% of celecoxib in a 1000 mL of 0.25% sodium dodecyl sulfate/pH 7 Sodium Phosphate Buffer solution under 50 RPM at 120 minutes after dissolution test.

Another composition of the invention comprises 65 mg to 160 mg celecoxib, 65 mg to 800 mg tartaric acid, 50 mg to 160 mg cross linked povidone, 50 mg to 160 mg cross linked carboxymethyl sodium cellulose, magnesium stearate and a small amount of PVPK30; wherein the formulation is in the form of granule, a capsule or a tablet, which has a dissolution rate of more than 60% of celecoxib in a 1000 mL of 0.25% sodium dodecyl sulfate/pH 7 Sodium Phosphate Buffer solution under 50 RPM at 120 minutes after dissolution test.

Another composition of the invention comprises 65 mg to 160 mg celecoxib, 65 mg to 800 mg succinic acid, 50 mg to 160 mg cross linked povidone, 50 mg to 160 mg cross linked carboxymethyl sodium cellulose, magnesium stearate and a small amount of PVPK30; wherein the formulation is in the form of granules, a capsule or a tablet, which has a dissolution rate of more than 60% of celecoxib in a 1000 mL of 0.25% sodium dodecyl sulfate/pH 7 sodium phosphate buffer solution under 50 RPM at 120 minutes after dissolution test.

Another composition of the invention comprises 65 mg to 160 mg celecoxib, 65 mg to 800 mg ascorbic acid, 50 mg to 160 mg cross linked povidone, 50 mg to 160 mg cross linked carboxymethyl sodium cellulose, magnesium stearate and a small amount of PVPK30; wherein the formulation is in the form of granules, a capsule or a tablet, which has a dissolution rate of more than 60% of celecoxib in a 1000 mL of 0.25% sodium dodecyl sulfate/pH 7 sodium phosphate buffer solution under 50 RPM at 120 minutes after dissolution test.

Another composition of the invention comprises 65 mg to 160 mg celecoxib, 65 mg to 800 mg citric acid, 50 mg to 160 mg cross linked povidone, 50 mg to 160 mg cross linked carboxymethyl sodium cellulose, magnesium stearate and a small amount of PVPK30; wherein the formulation is in the form of a granule, a capsule or a tablet, which has a dissolution rate of more than 60% of celecoxib in a 1000 mL of 0.25% sodium dodecyl sulfate/pH 7 sodium phosphate buffer solution under 50 RPM at 120 minutes after dissolution test.

Another composition of the invention comprises celecoxib, calcium carbonate, malic acid, cross linked carboxymethyl sodium cellulose, cross linked povidone, microcrystalline cellulose and magnesium stearate, wherein the formulation is in the form of a tablet, granules or a capsule.

Another composition of the invention comprises celecoxib, calcium carbonate, maleic acid, cross linked carboxymethyl sodium cellulose, cross linked povidone, microcrystalline cellulose and magnesium stearate, wherein the formulation is in the form of a tablet, granules or a capsule.

Another composition of the invention comprises celecoxib, calcium carbonate, tartaric acid, cross linked carboxymethyl sodium cellulose, cross linked povidone, microcrystalline cellulose and magnesium stearate, wherein the formulation is in the form of a tablet, granules or a capsule.

Another composition of the invention comprises celecoxib, calcium carbonate, citric acid, cross linked carboxymethyl sodium cellulose, cross linked povidone, microcrystalline cellulose and magnesium stearate, wherein the formulation is in the form of a tablet, granules or a capsule.

Another composition of the invention comprises celecoxib, calcium carbonate, succinic acid, cross linked carboxymethyl sodium cellulose, cross linked povidone, microcrystalline cellulose and magnesium stearate, wherein the formulation is in the form of a tablet, granules or a capsule.

Another composition of the invention comprises celecoxib, calcium carbonate, ascorbic acid, cross linked carboxymethyl sodium cellulose, cross linked povidone, microcrystalline cellulose and magnesium stearate, wherein the formulation is in the form of granules, a capsule or a tablet.

Selective COX-2 inhibitors such as celecoxib, etoricoxib and imrecoxib are used to treat osteoarthritis, rheumatoid arthritis, acute pain. Selective COX-2 inhibitors may be made into a weakly effervescent disintegrating formulation e.g., an orodispersible or orally disintegrating tablet comprising (or consisting essentially of) a pair of weak acid and a carbonate, and one or more excipients selected from a filling agent, a diluent, a lubricant and an expander. After the weakly effervescent disintegrating formulation of selective COX-2 inhibitors disintegrates in the neutral pure water, the pH of the suspension formed is greater than 4.9.

In another embodiment, the present invention relates to a weakly effervescent formulation which comprises calcium carbonate, one acid selected from malic acid, maleic acid, adipic acid, fumaric acid, tartaric acid, succinic acid and ascorbic acid, a selective COX-2 inhibitor selected from celecoxib, etoricoxib and imrecoxib, cross linked povidone, cross linked sodium carboxymethyl cellulose, microcrystalline cellulose, magnesium stearate and PEG 6000. After the weakly effervescent disintegrating formulation disintegrates in 200 mL of neutral pure water, the pH of the suspension formed is greater than 4.9, such that the formulation is in the form of a dispersible tablet.

Other embodiments of the present invention relate to a weakly effervescent formulation which comprises calcium carbonate, an acid selected from malic acid, maleic acid, succinic acid, adipic acid, fumaric acid, tartaric acid and ascorbic acid, and a selective COX-2 inhibitor selected from celecoxib, and imrecoxib. After the weakly effervescent disintegrating formulation of a selective COX-2 inhibitor disintegrates in neutral pure water, the pH of the suspension formed is more than 4.9, such that the formulation is in the form of an ODT or a dispersible tablet.

Another embodiment of the present invention relates to a weakly effervescent formulation which comprises calcium carbonate, malic acid, cross linked povidone, cross linked sodium carboxymethyl cellulose, microcrystalline cellulose, magnesium stearate, PEG 6000 and celecoxib. After the weakly effervescent formulation of celecoxib disintegrates in neutral pure water, the pH of the suspension formed is more than 4.9, such that the formulation is in the form of an ODT or a dispersible tablet.

Another embodiment of the present invention relates to a weakly effervescent formulation which comprises sodium hydrogen carbonate, monosodium tartrate, cross linked povidone, cross linked sodium carboxymethyl cellulose, microcrystalline cellulose, magnesium stearate, PEG 6000 and etoricoxib. After the weakly effervescent formulation of etoricoxib disintegrates in neutral pure water, the pH of the suspension formed is more than 4.9, such that the formulation is in the form of an ODT or a dispersible tablet.

Another embodiment of the present invention relates to a weakly effervescent formulation which comprises calcium carbonate, malic acid, cross linked povidone, cross linked sodium carboxymethyl cellulose, microcrystalline cellulose, magnesium stearate, PEG 6000 and imrecoxib. After the weakly effervescent formulation of imrecoxib disintegrates in neutral pure water, the pH of the suspension formed is more than 4.9, such that the formulation is in the form of an ODT or a dispersible tablet.

### Hormone Contraceptives

Hormonal contraceptives, such as desogestrel, norgestrel, levonorgestrel, megestrol, norethisterone, norgestimate, norethisterone oxime, ethinylestradiol, quinestrol, desethinylestradiol, megestrol acetate and progesterone, may be made into a weakly effervescent disintegrating formulation e.g., an orodispersible or orally disintegrating tablet or granules comprising (or consisting essentially of) a pair of weak acid and a carbonate, and one or more excipients selected from a filling agent, a diluent, a lubricant and an expander. After the weakly effervescent disintegrating formulation of hormone contraceptive disintegrates in the neutral water, the pH of the suspension or solution formed is greater than 4.9, such that the formulation is in the form of an ODT.

Other embodiments of the present invention relate to weakly effervescent formulations comprising sodium hydrogen carbonate, potassium hydrogen carbonate, a weak organic acid selected from monosodium malate, monosodium maleate, monosodium succinate, monosodium adipate, monosodium oxalate, monosodium citrate, monosodium tartrate, monopotassium malate, monopotassium maleate, monopotassium succinate, monopotassium adipate, monopotassium oxalate, monopotassium citrate, monopotassium tartrate, ascorbic acid, expander, diluent, filler, lubricant and a hormonal contraceptive selected from desogestrel, norgestrel, levonorgestrel, megestrol, norethisterone, norgestimate, quinestrol, norethisterone oxime, ethinylestradiol, desethinylestradiol, megestrol acetate, progesterone or a combination of any two or more thereof. After the weakly effervescent disintegrating formulation of hormonal contraceptive drug disintegrates in neutral pure water, the pH of the suspension formed is more than 4.9, such that the formulation is in the form of an ODT.

Another embodiment of the present invention relates to a weakly effervescent formulation which comprises calcium carbonate, malic acid, a hormone contraceptives selected from the group consisting of desogestrel, norgestrel, levonorgestrel, megestrol, norethisterone, norgestimate, norethisterone oxime, ethinylestradiol, quinestrol, desethinylestradiol, megestrol acetate and progesterone, cross linked povidone, cross linked sodium carboxymethyl cellulose, microcrystalline cellulose, magnesium stearate and PEG 6000. After the weakly effervescent disintegrating formulation disintegrates in 200 mL of neutral pure water, the pH of the suspension or solution formed is greater than 4.9, such that the formulation is in the form of an oral dispersible tablet.

Another embodiment of the present invention relates to a weakly effervescent formulation which comprises sodium hydrogen carbonate, monosodium tartrate, cross linked povidone, cross linked sodium carboxymethyl cellulose, microcrystalline cellulose, magnesium stearate, PEG 6000 and desogestrel. After the weakly effervescent formulation of desogestrel disintegrates in neutral pure water, the pH of the suspension formed is more than 4.9, such that the formulation is in the form of an ODT.

Another embodiment of the present invention relates to a weakly effervescent formulation which comprises sodium hydrogen carbonate, monosodium tartrate, cross linked povidone, cross linked sodium carboxymethyl cellulose, microcrystalline cellulose, magnesium stearate, PEG 6000 and norgestrel. After the weakly effervescent formulation of norgestrel disintegrates in neutral pure water, the pH of the suspension formed is more than 4.9, such that the formulation is in the form of an ODT.

Another embodiment of the present invention relates to a weakly effervescent formulation which comprises sodium hydrogen carbonate, monosodium tartrate, cross linked povidone, cross linked sodium carboxymethyl cellulose, microcrystalline cellulose, magnesium stearate, PEG 6000 and levonorgestrel. After the weakly effervescent formulation of levonorgestrel disintegrates in neutral pure water, the pH of the suspension formed is more than 4.9, such that the formulation is in the form of an ODT.

Another embodiment of the present invention relates to a weakly effervescent formulation which comprises sodium hydrogen carbonate, monosodium tartrate, cross linked povidone, cross linked sodium carboxymethyl cellulose, microcrystalline cellulose, magnesium stearate, PEG 6000 and norethisterone. After the weakly effervescent formulation of norethisterone disintegrates in neutral pure water, the pH of the suspension formed is more than 4.9, such that the formulation is in the form of an ODT.

Another embodiment of the present invention relates to a weakly effervescent formulation which comprises sodium hydrogen carbonate, monosodium tartrate, cross linked povidone, microcrystalline cellulose, magnesium stearate, PEG 6000 and megestrol. After the weakly effervescent formulation of megestrol disintegrates in neutral pure water, the pH of the suspension formed is more than 4.9, such that the formulation is in the form of an ODT.

Another embodiment of the present invention relates to a weakly effervescent formulation which comprises sodium hydrogen carbonate, monosodium tartrate, cross linked povidone, cross linked sodium carboxymethyl cellulose, microcrystalline cellulose, magnesium stearate, PEG 6000 and ethinylestradiol. After the weakly effervescent formulation of ethinylestradiol disintegrates in neutral pure water, the pH of the suspension formed is more than 4.9, such that the formulation is in the form of an ODT.

Another embodiment of the present invention relates to a weakly effervescent formulation which comprises sodium hydrogen carbonate, monosodium tartrate, cross linked povidone, microcrystalline cellulose, magnesium stearate, PEG 6000 and progesterone. After the weakly effervescent formulation of progesterone disintegrates in neutral pure water, the pH of the suspension formed is more than 4.9, such that the formulation is in the form of an ODT.

### Anti-Epileptics

In other embodiments, the API may be an anti-epileptic, such as carbamazepine, primidone, topiramate, phenytoin sodium, phenobarbital, sodium valproate, ethosuximide, lamotrigine or gabapentin.

Another composition of the invention comprises calcium carbonate, an organic acid selected from the group consisting of malic acid, maleic acid, succinic acid, tartaric acid, citric acid and ascorbic acid, an anti-epileptic drug consisting of phenobarbital, carbamazepine, topiramate, ethosuximide, gabapentin, phenytoin sodium and sodium valproate. The composition further comprises cross linked povidone, microcrystalline cellulose, and magnesium stearate. After the composition of the invention disintegrates in neutral pure water, the pH of the suspension formed is more than 4.9, such that the formulation is in the form of an ODT or a dispersible tablet.

Another composition of the invention comprises phenobarbital, calcium carbonate, cross linked povidone, microcrystalline cellulose, and an acid selected from the group consisting of malic acid, maleic acid, succinic acid, tartaric acid, citric acid and ascorbic acid. After the composition of the phenobarbital disintegrates in neutral pure water, the pH of the suspension formed is more than 4.9, such that the formulation is in the form of an ODT or a dispersible tablet.

Another composition of the invention comprises phenobarbital, calcium carbonate, malic acid, cross povidone, and magnesium stearate. After the composition of the phenobarbital disintegrates in neutral pure water, the pH of the suspension formed is more than 4.9, such that the formulation is in the form of an ODT or a dispersible tablet.

Another composition of the invention comprises carbamazepine, calcium carbonate, and an acid selected from the group consisting of malic acid, maleic acid, succinic acid, tartaric acid, citric acid and ascorbic acid. After the composition of the invention disintegrates in neutral pure water, the pH of the suspension formed is more than 4.9, such that the formulation is in the form of an ODT or a dispersible tablet.

Another composition of the invention comprises calcium carbonate, an acid selected from malic acid, maleic acid, succinic acid, tartaric acid, citric acid, ascorbic acid, ethosuximide, cross povidone, and magnesium stearate. After the composition of the ethosuximide disintegrates in neutral pure water, the pH of the suspension formed is more than 4.9, such that the formulation is in the form of an ODT or a dispersible tablet.

Another composition of the invention comprises calcium carbonate, an acid selected from malic acid, maleic acid, succinic acid, tartaric acid, citric acid, ascorbic acid, topiramate, cross linked povidone, and magnesium stearate. After the composition of the topiramate disintegrates in neutral pure water, the pH of the suspension formed is more than 4.9, such that the formulation is in the form of an ODT or a dispersible tablet.

Another composition of the invention comprises calcium carbonate, an acid selected from malic acid, maleic acid, succinic acid, tartaric acid, ascorbic acid and citric acid, gabapentin, cross linked povidone, and magnesium stearate. After the composition of the gabapentin disintegrates in neutral pure water, the pH of the suspension formed is more than 4.9, such that the formulation is in the form of an ODT or a dispersible tablet.

Another composition of the present invention comprises calcium carbonate, an acid selected from malic acid, tartaric acid, succinic acid, maleic acid, ascorbic acid and citric acid, cross linked povidone and sildenafil such that the formulation is in the form of an ODT and produces a pH of more than 4.9, after disintegrating in 200 mL of neutral water.

In other aspects, the present invention is directed to a method for the oral administration of a drug without water or other drinking fluid, or with only a small volume of water or other drinking fluid. In certain preferred embodiments, the method comprises administration of an oral disintegration tablet or a dispersible tablet. In certain preferred embodiments, the method comprises administration of a stable form of an oral disintegration tablet or a dispersible tablet. The drug may be administered in the form of a weakly effervescent tablet according to the present invention, including an orally disintegrating tablet or a dispersible tablet. In a preferred embodiment, the weakly effervescent tablet according to the present invention is an orally disintegrating tablet.

In one or more embodiments, the weakly effervescent tablet according to the present invention may comprise an API, vitamins, mineral, food nutrients, a pair of weak acids and weak bases, a physical disintegration excipient, expander, binder, filling agent, sweetener, and a lubricant.

In one or more embodiments, the weak base is a carbonate salt, such as sodium bicarbonate, calcium carbonate, potassium bicarbonate, lithium bicarbonate or magnesium carbonate.

In accordance with embodiments of the invention, the weak acid/weak base couple or pair react in an aqueous medium to produce a sufficient amount of gas to disintegrate the tablet, preferably within a relatively short period of time. The disintegration time will be influenced by the resultant force produced by gas and the size of the tablet. In various embodiments, the disintegration time may be within about 1 minute, or within about 1.5 minutes, or within about 2 minutes, or within about 2.5 minutes, or within about 3 minutes. After disintegration of the weakly effervescent disintegrating tablet of the invention in neutral pure water, the pH of the suspension or solution is greater than 4.9, preferably within the range pH 5 to 7.

In certain embodiments of the present invention, when the pharmaceutical excipient comprises hydrogen carbonate, the organic acid is a weak acid selected from monosodium malate, monosodium maleate, monosodium succinate, monosodium adipate, monosodium fumarate, monosodium oxalate, monosodium citrate, monosodium tartrate, monopotassium malate, monopotassium maleate, monopotassium succinate, monopotassium adipate, monopotassium oxalate, monopotassium citrate, monopotassium tartrate, or ascorbic acid. In preferred embodiments, the other excipients are cross linked povidone, cross linked sodium carboxymethyl cellulose, microcrystalline cellulose, aspartame, sucralose, menthol, magnesium stearate, PEG 6000, and colloidal silicon dioxide or combinations thereof.

In certain embodiments of the present invention, when the pharmaceutical excipient comprises calcium carbonate, the organic acid is selected from malic acid, maleic acid, succinic acid, adipic acid, ascorbic acid, fumaric acid and tartaric acid in the mole ratio range of binary acid to calcium carbonate from 0.35:1.0 to 1.25:1.0 or from 0.15:1.0 to 1.0:1.0.

In certain embodiments of the present invention, when the pharmaceutical excipient comprises calcium carbonate, the organic acid is a small amount of ascorbic acid, the mole ratio range of ascorbic acid to calcium carbonate is from 0.5:1.0, 1.0:1.0 to 1.75:1.0 or 1.50:1.0.

In preferred embodiments, the other excipients may be selected from cross linked povidone, cross linked sodium carboxymethyl cellulose, microcrystalline cellulose, aspartame, peppermint, colloidal silicon dioxide, magnesium stearate, and PEG 6000, or combinations thereof.

In another aspect, the present invention is directed to a method of making a weakly effervescent pharmaceutical formulation. The method comprises making acidic granules, preferably weakly acidic granules, and basic granules or powder.

In preferred embodiments, the weakly acidic granules may be made by mixing a weak acid selected from monosodium malate, monosodium maleate, monosodium succinate, monosodium adipate, monosodium fumarate, monosodium oxalate, monosodium citrate, monosodium tartrate, monopotassium malate, monopotassium maleate, monopotassium succinate, monopotassium adipate, monopotassium oxalate, monopotassium citrate, monopotassium tartrate, and ascorbic acid with an acidic API, acidic vitamins, minerals, cross linked povidone, cross linked sodium carboxymethyl cellulose, microcrystalline cellulose, and aspartame. The mixture may then then be formulated into acidic granules within a fluid bed or wet granulator, for example, through the spraying of PVPK30 solution.

In preferred embodiments, the basic granules may be made by mixing: a carbonate salt, such as carbonate salt of calcium, magnesium, potassium, sodium, lithium; or a bicarbonate salt, such as bicarbonate of sodium, potassium, lithium with a basic API, aspartame, and peppermint, or combinations thereof. Both the acidic and basic granules may then be mixed together with one or more other excipients, such as colloidal silicon dioxide, magnesium stearate, and PEG 6000, and compressed into an ODT or a dispersible tablet.

In another aspect, the present invention is directed to a method of production of a high dissolution composition comprising the celecoxib. The method comprises:
1. Mixing 1 part of celecoxib with 1-6 parts of an organic acid selected from the group consisting of ascorbic acid, malic acid, maleic acid, succinic acid, tartaric acid and citric acid or a mixture thereof, with an alcohol selected from ethanol and benzyl alcohol until the celecoxib and the organic acid is fully dissolved.
2. 1 part of cross linked povidone, 1 part of cross linked sodium carboxymethyl cellulose is place in a fluid bed or granulator with a small amount of sodium dodecyl sulfate (SDS).
3. The alcoholic solution of process 1 above containing celecoxib and organic acid is sprayed into the fluid bed or granulator.
4. A small amount of aqueous 8% PVPK30 is sprayed into the fluid bed until the size of granules is about 24 mesh. The granules made are dried by hot air; and the granules are mixed with magnesium stearate and is compressed into a tablet or filled into a capsule or granules bag.

### Definitions

In order for the invention described herein to be more fully understood, the following definitions are provided for the purposes of this disclosure.

The term “comprising” is an inclusive term interpreted to mean containing, embracing, covering or including the elements listed following the term, but not excluding other unrecited elements.

The term “consisting essentially of” means the list of elements may include additional

components or excipients that do not materially contribute to the working of the invention.

The term “consisting of” is an exclusive term and means consisting only of.

The term “therapeutically effective amount” or “effective amount” means an amount that, when administered to an animal for treating a disease, disorder or condition, is sufficient to produce a desired therapeutic effect (e.g., to affect treatment for that disease).

The terms “composition” and “formulation” are synonymous and are used interchangeably herein.

1000 mL of 0.25% sodium dodecyl sulfate/pH 7 sodium phosphate buffer solution is a 1000 mL of pH 7 sodium phosphate buffer solution containing 2.5 grams of sodium dodecyl sulfate.

The terms “dissolution” and “dissociation” are synonymous and are used interchangeably herein.

The term “PVP” refers to polyvinyl pyrrolidone.

The term “RPM” refers to rotations per minute.

Pellet=sphero, a small ball.

### BRIEF DESCRIPTION OF THE FIGURES

FIG. 1 is a graph showing celecoxib plasma concentration in a male beagle dog (subject A1) after being administered a 150 mg celecoxib tablet of the invention in comparison to a control, i.e., a 200 mg CELEBREX capsule manufactured by Pfizer, as described in Example 16.

FIG. 2 is a graph showing celecoxib plasma concentration in a male beagle dog (subject A2) after being administered a 150 mg celecoxib tablet of the invention in comparison to a control, i.e., a 200 mg CELEBREX capsule manufactured by Pfizer, as described in Example 16.

FIG. 3 is a graph showing celecoxib plasma concentration in a female beagle dog (subject B1) after being administered a 150 mg celecoxib tablet of the invention in comparison to a control, i.e., a 200 mg CELEBREX capsule manufactured by Pfizer, as described in Example 16.

FIG. 4 is a graph showing celecoxib plasma concentration in a female beagle dog (subject B2) after being administered a 150 mg celecoxib tablet of the invention in comparison to a control, i.e., a 200 mg CELEBREX capsule manufactured by Pfizer, as described in Example 16.

FIG. 5 is a graph showing mean celecoxib plasma concentration of four beagle dogs (subjects A1, A2, B1 and B2) after being administered a 150 mg celecoxib tablet of the invention in comparison to a control, i.e., a 200 mg CELEBREX capsule manufactured by Pfizer, as described in Example 16.

### DETAILED DESCRIPTION OF THE INVENTION

The invention will be described with reference to various specific and preferred embodiments and techniques. However, it should be understood that many variations and modification may be made while remaining within the spirit and scope of the invention.

### Tablet Composition

The composition of the invention comprising high dissolution cyclooxygenase-2 (COX-2) inhibitors contains celecoxib or imrecoxib, and an organic acid. The organic acid is selected from malic acid, ascorbic acid, maleic acid, succinic acid, tartaric acid and citric acid. The amount of organic acid is much higher than the amount of celecoxib or imrecoxib, and the ratio between them is from about 1:1 to 6:1. The greater the amount of organic acid, the higher the dissolution of celecoxib or imrecoxib. The composition of the invention may be in the form of granules, a tablet or a capsule. The composition further contains cross linked povidone, cross linked sodium carboxymethyl cellulose, a small amount of polyvinyl pyrrolidone such as PVPK30, magnesium stearate or combinations thereof. The celecoxib of the composition has higher dissolution of celecoxib in the aqueous medium or in the alimentary system and will increased absorption in human being if ingested by human being compared with celecoxib capsules on the market.

The composition of celecoxib with an organic acid may have calcium carbonate as a disintegrating agent or chemical expander; such a composition may be termed the weakly effervescent disintegration formulation. On the other hand, the weakly effervescent disintegration formulation may contains both a pair of base and acid with another API. The addition of the calcium carbonate in the composition will speed up the disintegration of the tablet. The composition of celecoxib with an organic acid and calcium carbonate will increase both the dissolution of celecoxib and calcium ion.

The weakly effervescent formulation of the invention may be prepared in a variety of ways known to those skilled in the art.

Examples of pharmaceutically acceptable gas-producing carbonates includes calcium carbonate, magnesium carbonate, potassium carbonate, potassium bicarbonate, sodium carbonate, sodium bicarbonate, lithium carbonate, lithium bicarbonate, etc.

Examples of weak acids include monosodium malate, monosodium maleate, monosodium succinate, monosodium adipate, monosodium fumarate, monosodium oxalate, monosodium citrate, monosodium tartrate, monopotassium malate, monopotassium maleate, monopotassium succinate, monopotassium adipate, monopotassium oxalate, monopotassium citrate, monopotassium tartrate, ascorbic acid, malic acid, maleic acid, succinic acid, tartaric acid and citric acid.

The composition of calcium carbonate with an organic acid may increase the dissociation of calcium ions and increase its absorption in the alimentary system because the organic acid will decompose the calcium carbonate to calcium ions which will combine with the acid to form a water soluble calcium salt of binary acid, such as calcium malate, calcium maleate and carbon dioxide which will evaporate from the aqueous medium. Such formulation have the CO₂ as a chemical expander or a disintegrating agent and this formulation may be called the weakly effervescent disintegration formulation if the amount of an organic is moderate and the final pH of the solution of disintegration is more than 4.9 in 200 mL of neutral water.

In other embodiments containing calcium carbonate and magnesium carbonate, the weak acid may be malic acid, maleic acid, succinic acid, adipic acid, fumaric acid and tartaric acid. Preferably, these acids are used in small amounts in the mole ratio range of binary acid to calcium carbonate from 0.35:1.0 to 1.0:1.0.

In certain embodiments, the monosodium salt of binary acids may not be strong enough to react with calcium carbonate and it may be appropriate to use a moderate amount of binary acid relative to calcium carbonate to cause the reaction of calcium carbonate to occur. The pH of the solution of the final reaction will limit the speed or violence of the reaction. Preferably, the mole ratio range of binary acid to calcium carbonate is from 0.35:1.0 to 1.0:1.0. In preferred embodiments, the mole ratio is 0.35:1.0 to 1.0:1.0. (This molar ratio range is calculated on the basis that 1 mmol of ascorbic acid=176 mg, 1 mmol of calcium carbonate=100 mg).

The pH of the solution of the final reaction will limit the speed or violence of the reaction. When the pH of the solution of the final reaction is more than 4.9, the amount of the acid added is appropriate.

Examples of expander or physically disintegration excipients include cross linked povidone, cross linked sodium carboxymethyl cellulose, polyvinylpyrrolidone, low substituted cellulose, sodium carboxymethyl starch, hydroxypropyl starch, among others.

Examples of the filler, lubricant, sweetener include microcrystalline cellulose, aspartame, peppermint, colloidal silicon dioxide, magnesium stearate, PEG 6000 or combinations thereof.

In certain preferred embodiments, the formulation of the invention comprises calcium carbonate together with ascorbic acid, binary acid and other common excipients. Ideally, this will promote the gas-producing reaction and generation of carbon dioxide, but also prevent burning of the mouth. After the weakly effervescent fast disintegrating tablets disintegrates in neutral water (preferably neutral pure water), the pH of the suspension or mixture formed is greater than 4.9. Maintaining the pH above 4.9 controls the rate of the reaction and prevents burning of the oral cavity.

In certain other preferred embodiments, the weakly effervescent fast disintegrating formulation comprises hydrogen carbonate together with a weak acid selected from monosodium malate, monosodium maleate, monosodium succinate, monosodium adipate, monosodium fumarate, monosodium oxalate, monosodium citrate, monosodium tartrate, monopotassium malate, monopotassium maleate, monopotassium succinate, monopotassium adipate, monopotassium oxalate, monopotassium citrate, monopotassium tartrate. A benefit of using a weak acid is to prevent the occurrence of burning in the mouth. After the weakly effervescent tablet disintegrates in the neutral water, the pH of the suspension formed is more than 4.9, such that the formulation is in the form of an ODT or a dispersible tablet.

Advantageously, the weakly effervescent tablet formulation in the form of an ODT or a dispersible tablet may be administered in the oral cavity of the patients without water. Preferably, the weakly effervescent tablet formulation partially or wholly disintegrates within about 3 minutes, or within about 2.5 minutes, or within about 2 minutes, or within about 1.5 minutes, or within about 1 minute. The time for dissolution may depend on the size of the tablet.

An advantage of preferred embodiments of the present invention is the prevention or mitigation of the occurrence of side effects of aspiration because the tablet may partially disintegrate within 1 minute, or totally disintegrate within 1 minute, even when the tablet weight is more than 1 gram or more than 2 grams.

Calcium is a mineral that makes up bones, as well as a salt that dissolves in our blood and regulates bodily function. The normal range for blood calcium level of most people is 8.6 to 10.3 mg/dL. The human body has about 3000 mL to 4500 mL of blood, and the maximum amount of blood calcium the body may have is therefore 450 mg. Many calcium carbonate tablets in the market have a strength of 1500 mg, which corresponds to about 600 mg of calcium ion. If 600 mg of calcium ions is absorbed into the body, it will lead to hypercalcemia, constipation, kidney stones, and potentially death of patients. Importantly, the absorption of calcium within a calcium carbonate tablet on the market is low and most of the calcium carbonate precipitates on the surface of the large intestine mucosa and causes constipation, or is passed through the digestive system.

Calcium carbonate tablets available on the market may have 100% dissolution in 900 mL of pH 1.2 HCl solution, but have dissolution of only 3.2% in 20 mL of pH 1.2 solution. Most people do not have more than 20 mL of gastric acid in an empty stomach. As a consequence, the calcium ions of ordinary calcium carbonate tablets cannot be dissolved completely in the stomach and the absorption of the calcium is low. The manufacturer of products on the market may increase the dosage of calcium carbonate in each tablet. However, as noted above, a high dosage of calcium carbonate may cause adverse effects such as hypercalcemia or constipation.

A product currently on the market is a calcium carbonate/vitamin C effervescent tablet known as CalVive C effervescent tablet. The CalVive C tablet is formulated for once daily administration and contains 950 mg of vitamin C and 247 mg of calcium ion. The weight of tablet is about 7.01 gram and the diameter of the tablet is 3.3 cm. The time of disintegration of CalVive C tablet is 130 seconds in 900 mL of pH 7 pure water and the pH of the solution formed after disintegration is 4.48. The dissolution of the calcium carbonate/vitamin C effervescent tablet is 97% in 900 mL of pure water of pH 7. The other ingredients in the tablet are citric acid, vitamin C, sodium bicarbonate, calcium lactate gluconate, calcium carbonate. The tablet is formulated to be taken after dissolving in 200 mL of water. The main effervescent agent is citric acid and sodium carbonate. In addition, the tablet contains a large amount of calcium derived from calcium lactate gluconate, which is water soluble.

In contrast, an advantageous embodiment of the present invention is that the calcium only comes from calcium carbonate which acts both as the API and gas producing effervescent agent. The calcium malate formed is water soluble and is different from calcium citrate which is slightly soluble in water. The invention does not comprise citric acid because it is a ternary acid and is a strong acid. In contrast to calcium tablets available on the market, the calcium carbonate weakly effervescent tablet according to the present invention may be an ODT or a dispersible tablet. In preferred embodiments, calcium tablets according to the present invention may partially disintegrate within about 1 minute, or totally disintegrate within about 3 minutes.

In an embodiment, weakly effervescent tablets according to the present invention contain 75 mg calcium carbonate (which equates to 30 mg calcium ion) and the dissolution of the tablet in 900 mL of neutral pure water is 65-75%. Advantageously, such tablets may release about 20 mg of calcium ions in the digestive system. In contrast, an ordinary calcium carbonate tablet may contain 1000 mg calcium carbonate (equating to 400 mg calcium ion). In 900 mL of pH 7 (neutral) water the dissolution of the calcium of the ordinary tablet is less than 1.5%, which means that only 6 mg of the calcium ions is released to be absorbed in the alimentary system. The remainder of the calcium carbonate of the ordinary tablet is passed out of the alimentary system and may induce constipation. In addition, if a person has a calcium carbonate intake of 1500 mg per day, and if the calcium carbonate consumed has a dissolution of 50%, the calcium ions dissolved or absorbed may be 300 mg, which may cause hypercalcemia of the patient.

The calcium carbonate and vitamin C (ascorbic acid) in the weakly effervescent tablets according to the present invention may be an ODT or a dispersible tablet. In preferred embodiments, the weakly effervescent tablet may partially disintegrate within about 1 minute or totally disintegrate within about 3 minutes and have a dissolving rate of more than 20% and less than 75% in 900 mL of pH 7 pure water.

In a preferred embodiment, the present invention relates to a weakly effervescent disintegration formulation comprising less than 75 mg of calcium carbonate per tablet each day, which has a range of dissolution of calcium ions from 20 to 75%.

In another aspect the present invention relates to a method of treating hypocalcaemia or calcium deficiency in a subject, the method comprising administering to a subject in need thereof a weakly effervescent disintegration formulation comprising less than 75 mg of calcium carbonate per dosage form each day, which has a range of dissolution of calcium ions from 20 to 75%.

In an embodiment, the dosage form is an oral disintegration tablet, a dispersible tablet, a sublingual tablet, a tablet, a capsule or granules.

### General Methods for Tablet Manufacture

The following embodiments illustrate the method of production of a high dissolution composition comprising the celecoxib, imrecoxib, or calcium carbonate and methods for manufacturing a weakly effervescent formulation in accordance with the present invention.

### Embodiment 1

Method of production of celecoxib composition with organic acid is described as follows:
A. 1 part of celecoxib and 1 to 6 parts of organic acid selected from maleic acid, malic acid, tartaric acid, succinic acid, citric acid and ascorbic acid is dissolved in 10 to 20 parts of liquid alcohol selected from a group consisting of ethanol and benzyl alcohol.
B. Granulation: 1 part of cross linked povidone, 1 part of cross linked carboxymethyl sodium cellulose, 0.1 part of sodium dodecyl sodium is put in the fluid bed;
C. The ethanol solution containing celecoxib and organic acid of process 1 is sprayed into the fluid bed. Then the powder is made into granules with 8% PVPK30 in fluid bed and dried until the water content is below 4%. The whole process of granulation may be made in the granulator using other methods. The above granules and magnesium stearate are mixed and then pressed into a tablet.
D. The dissolution of the celecoxib of the above composition is more than 60% in 1000 mL of pH 7 sodium phosphate buffer solution containing 2.5 gram of SDS and the pH is in the range of 2.7-3.2.
E. The granule of C may be mixed with granules of calcium carbonate and then is compressed into tablet or filled into a capsules containing celecoxib, organic acid and calcium carbonate.

### Embodiment 2

A weakly effervescent tablet of calcium carbonate and vitamin C is manufactured using two processes of granulation:
1^{st} granulation: calcium carbonate is mixed with cross linked povidone, cross linked carboxymethyl sodium cellulose, microcrystalline cellulose and sprayed with 8% of PVPK30 in wet granulator or fluid bed, and the wet granule is dried in an oven or fluid bed until the water content is below 4%.
2^{nd} granulation: ascorbic acid is mixed with cross linked povidone, microcrystalline cellulose, aspartame, and sprayed with 8% PVPK30 in wet granulator or fluid bed, and the wet granule is dried in an oven or fluid bed until the water content is below 4%.
The two kinds of granules prepared above are mixed with magnesium stearate and sprayed with peppermint ethanol solution and then pressed into a tablet. After the weakly effervescent tablet disintegrates in the neutral water, the pH of the suspension formed is 6.32, which is more than 4.9, such that the formulation is a dispersible tablet or an ODT, preferably if the tablet size is small.

Table 1 below shows the results for tablets prepared according to Embodiment 2 and Embodiment 3 of the present invention versus a CaCO₃/Vitamin D₃ tablet from another manufacturer in 900 mL of pH 7 pure water for comparison:

TABLE 1
CaCO₃/vitamin C and CaCO₃/malic acid tablet
compared with CaCO₃/Vitamin D₃ (Comparative),
as described in paragraph [00444]

| | 5 mins/ | 10 mins/ | 20 mins/ | 30 mins/ |
|---|---|---|---|---|
| | calcium | calcium | calcium | calcium |
| | dissolving | dissolving | dissolving | dissolving |
| CaCO₃/vitamin C | 33.97% | 36.65% | 40.54% | 42% |
| tablet | | | | |
| CaCO₃/malic acid | 23.32% | 34.69% | 46.64% | 58.90% |
| tablet | | | | |
| CaCO₃/Vitamin D₃ | 0.92% | 0.93% | 0.98% | 1.11% |
| (Comparative) | | | | |

### Embodiment 3

A weakly effervescent tablet of calcium carbonate may be manufactured using two processes of granulation:
Granulation 1: calcium carbonate is mixed with cross linked povidone, cross linked carboxymethyl sodium cellulose, microcrystalline cellulose and sprayed with 8% PVPK30 in fluid bed, then the granule is dried until the water content is below 4%.
Granulation 2: one organic acid selected from malic acid, maleic acid, succinic acid, adipic acid, citric acid, fumaric acid and tartaric acid is mixed with cross povidone, microcrystalline cellulose, and aspartame, and sprayed with 8% PVPK30 in fluid bed, then the granule is dried until the water content is below 4%.
The two kinds of granules prepared above and magnesium stearate are mixed and then sprayed with peppermint ethanol solution and then pressed into a tablet. After the weakly effervescent tablet prepared according to the invention disintegrates in neutral water, the pH of the suspension formed is 5.99 and is more than 4.9, such that the formulation is in the form of a dispersible tablet. From the table above, the result is faster in dissolution compared to ordinary tablets of calcium carbonate. The addition of small amounts of binary acid in the tablet of calcium carbonate may greatly raise the dissolution of the calcium ions in the stomach.

The experiments in Embodiment 2 and Embodiment 3 show that the addition of ascorbic acid could decrease the time of disintegration, and the addition of ascorbic acid in the formulation may increase the dissolution of the calcium ions in the pH 7 pure water. The experiment showed that the calcium carbonate and ascorbic acid formulation according to the present invention had greater dissolving rate than ordinary calcium carbonate tablet. The calcium carbonate and ascorbic acid complex has a shorter disintegration time than ordinary calcium carbonate.

### Embodiment 4

A weakly effervescent tablet of calcium carbonate with vitamin C and glucosamine salt is manufactured using two processes of granulation:
Granulation 1: calcium carbonate is mixed with cross linked povidone, microcrystalline cellulose and sprayed with 8% PVPK30 in fluid bed, then the granule is dried until the water content is below 4%.
Granulation 2: ascorbic acid is mixed with glucosamine HCl, cross linked povidone, microcrystalline cellulose, aspartame and sprayed with 8% PVPK30 in fluid bed, then the granule is dried until the water content is below 4%.
The two kinds of the above granules and magnesium stearate are mixed and then sprayed with peppermint ethanol solution and then pressed into a tablet. After the weakly effervescent tablet disintegrates in the neutral water, the pH of the suspension formed is more than 4.9, such that the formulation is in the form of a dispersible tablet.

Dissolution test of a tablet composition containing 25 mg calcium carbonate, 50 mg vitamin C and 500 mg glucosamine HCl in 900 mL of neutral pure water under stirring at a speed of 75 RPM when measured at 30 min, as shown in table 2.

TABLE 2
Dissolution of tablet, as described in paragraph [00448]

| Time (min.) | 20 | 60 |
|---|---|---|
| Dissolution % | 69% | 77.6% |
| pH | | 6.27 |

### Embodiment 5

A weakly effervescent tablet of calcium carbonate with vitamins such as vitamin B1, vitamin B2, vitamin B6, vitamin B12, vitamin C, vitamin A, vitamin D, folic acid, vitamin E, vitamin K, nicotinic acid, folic acid, pantothenic acid, choline, biotin and calcium carbonate is manufactured using two processes of granulation:
Granulation 1: calcium carbonate is mixed with cross povidone, vitamin B1, vitamin B2, vitamin B6, vitamin B12, folic acid, nicotinic acid, pantothenic acid, choline, biotin, vitamin A powder, vitamin D powder, microcrystalline cellulose and sprayed with 8% PVPK30 in wet granulator, the wet granule is dried in an oven until the water content is below 4%.
Granulation 2: ascorbic acid is mixed with cross povidone, microcrystalline cellulose, and aspartame, and sprayed with 8% PVPK30 in fluid bed. Then the vitamin E and vitamin K in ethanol is sprayed into the fluid bed.
Both kinds of the above granules and magnesium stearate are mixed and then sprayed with peppermint ethanol solution and then pressed into a tablet. After the weakly effervescent tablet disintegrates in the neutral water, the pH of the suspension formed is more than 4.9, such that the formulation is in the form of a dispersible tablet.

### Embodiment 6

A weakly effervescent tablet of calcium carbonate with vitamins and minerals such as vitamin B1, vitamin B2, vitamin B6, vitamin B12, vitamin C, vitamin A, vitamin D, vitamin E, vitamin K, nicotinic acid, folic acid, pantothenic acid, choline, biotin, calcium carbonate, iron sulfate, copper sulfate, zinc sulfate, magnesium sulfate, potassium chloride, sodium chloride is manufactured using two processes of granulation:
Granulation 1: calcium carbonate is mixed with cross linked povidone, vitamin B1, vitamin B2, vitamin B6, vitamin B12, vitamin A, vitamin D, folic acid, nicotinic acid, pantothenic acid, choline, biotin, iron sulfate, copper sulfate, zinc sulfate, magnesium sulfate, potassium chloride, sodium chloride, microcrystalline cellulose and sprayed with 8% PVPK30 in wet granulator, the wet granule is dried in an oven until the water content is below 4%.
Granulation 2: ascorbic acid is mixed with cross linked povidone, microcrystalline cellulose, aspartame and sprayed with 8% PVPK30 in fluid bed. Then the vitamin E and vitamin K in ethanol is sprayed into the fluid bed.
Both kinds of the above granules and magnesium stearate are mixed and then sprayed with peppermint ethanol solution and then pressed into a tablet. After the weakly effervescent tablet disintegrates in the neutral water, the pH of the suspension formed is more than 4.9, such that the formulation is in the form of a dispersible tablet.

### Embodiment 7

A weakly effervescent disintegrating tablet of an anti-epileptic drug such as phenytoin sodium, phenobarbital, sodium valproate, ethosuximide (pKa 10.73), lamotrigine (pKa 15), gabapentin (pKa 4.63), may be manufactured using two processes of granulation:
1st granulation: a bicarbonate of sodium or potassium is mixed with one of phenytoin sodium, phenobarbital, sodium valproate, ethosuximide, lamotrigine, gabapentin, 5% of cross linked povidone of the weight of the tablet, 5% of cross linked carboxymethyl sodium cellulose of the weight of the tablet, 5% of microcrystalline cellulose of the weight of the tablet, and sprayed with 8% PVPK30 in fluid bed, the granulation is continued until the size of the granule is bigger than 40 mesh, then dried until the water content is below 4%.
2nd granulation: one weak acid selected from the group consisting of monosodium malate, monosodium maleate, monosodium succinate, monosodium adipate, monosodium oxalate, monosodium citrate, monosodium tartrate, ascorbic acid, monopotassium malate, monopotassium maleate, monopotassium succinate, monopotassium adipate, monopotassium oxalate, monopotassium citrate, monopotassium tartrate, is mixed with about 5% of cross linked povidone of the weight of the tablet, 5% of cross linked carboxymethyl sodium cellulose of the weight of the tablet, 5% of microcrystalline cellulose of the weight of the tablet, aspartame and sprayed with 8% PVPK30 in fluid bed, the granulation is continued until the size of the granule is bigger than 40 mesh, then dried until the water content is below 4%.
Both kinds of the above granules and magnesium stearate are mixed together and then sprayed with peppermint ethanol solution and then pressed into a tablet. After the resultant weakly effervescent tablet disintegrates in the neutral water, the pH of the suspension formed is more than 4.9, such that the formulation is in the form of an ODT or a dispersible tablet.

### Embodiment 8

A weakly effervescent disintegrating tablet of a sartan API such as losartan, candesartan, valsartan, telmisartan, fimasartan, irbesartan is manufactured using two processes of granulation:
1st granulation: a bicarbonate of sodium or potassium is mixed with about 5% of cross linked povidone of the weight of the tablet, 5% of cross linked carboxymethyl sodium cellulose of the weight of the tablet, 5% of microcrystalline cellulose of the weight of the tablet, aspartame and one sartan selected from the group consisting of losartan, candesartan, valsartan, telmisartan, fimasartan and irbesartan, and sprayed with 8% PVPK30 in fluid bed, the granulation is continued until the size of the granule is bigger than 40 mesh, then dried until the water content is below 4%.
2nd granulation: one weak acid was selected from the group consisting of monosodium malate, monosodium maleate, monosodium succinate, monosodium adipate, monosodium oxalate, monosodium citrate, monosodium tartrate, ascorbic acid, monopotassium malate, monopotassium maleate, monopotassium succinate, monopotassium adipate, monopotassium oxalate, monopotassium citrate, monopotassium tartrate or a mixture thereof, is mixed with about 5% of cross linked povidone of the weight of the tablet, 5% of cross linked carboxymethyl sodium cellulose of the weight of the tablet, 5% of microcrystalline cellulose of the weight of the tablet, aspartame and sprayed with 8% PVPK30 in fluid bed, the granulation is continued until the size of the granule is bigger than 40 mesh, then dried until the water content is below 4%.
Both kinds of the above granules and magnesium stearate are mixed and then sprayed with peppermint ethanol solution and then pressed into a tablet. After the invention of weak effervescent tablet disintegrates in the neutral water, the pH of the suspension formed is more than 4.9, such that the formulation is in the form of an ODT or a dispersible tablet.

### Embodiment 9

A weakly effervescent disintegrating tablet of a selective COX-2 inhibitor such as celecoxib, etoricoxib, imrecoxib is manufactured using two processes of granulation:
1st granulation: calcium carbonate is mixed with about 5% of cross linked povidone of the weight of the tablet, 5% of cross linked carboxymethyl sodium cellulose of the weight of the tablet, 5% of microcrystalline cellulose of the weight of the tablet, aspartame and sprayed with 8% PVPK30 in fluid bed, the granulation is continued until the size of the granule is bigger than 40 mesh, then dried until the water content is below 4%.
2nd granulation: A COX-2 inhibitor of celecoxib or imrecoxib and one acid selected from malic acid, maleic acid, adipic acid, fumaric acid, tartaric acid, succinic acid and ascorbic acid is mixed with about 5% of cross linked povidone of the weight of the tablet, 5% of cross linked carboxymethyl sodium cellulose of the weight of the tablet, 5% of microcrystalline cellulose of the weight of the tablet, and aspartame, and sprayed with 8% PVPK30 in fluid bed, the granulation is continued until the size of the granule is bigger than 40 mesh, then dried until the water content is below 4%.
Both kinds of the above granules and magnesium stearate are mixed and then sprayed with peppermint ethanol solution and pressed into a tablet. After the weakly effervescent tablet disintegrates in neutral water, the pH of the suspension formed is more than 4.9, such that the formulation is in the form of an ODT or a dispersible tablet.

### Embodiment 10

A weakly effervescent disintegrating tablet of an alpha-glucosidase inhibitor such as miglitol (miglibose), acarbose, voglibose is manufactured using two processes of granulation:
1st granulation: calcium carbonate is mixed with about 5-10% of cross linked povidone of the weight of the tablet, 5-10% of cross linked carboxymethyl sodium cellulose of the weight of the tablet, 5% of microcrystalline cellulose of the weight of the tablet and one alpha-glucosidase inhibitor selected from miglitol (miglibose), acarbose and voglibose, aspartame and sprayed with 8% PVPK30 in fluid bed, the granulation is continued until the size of the granule is bigger than 40 mesh, then dried until the water content is below 4%.
2nd granulation: one weak acid of malic acid, maleic acid, adipic acid, fumaric acid, tartaric acid, succinic acid, ascorbic acid, citric acid and mixtures thereof is mixed with about 5-10% of cross linked povidone of the weight of the tablet, 5-10% of cross linked carboxymethyl sodium cellulose of the weight of the tablet, 5% of microcrystalline cellulose of the weight of the tablet, and aspartame, and sprayed with 8% PVPK30 in fluid bed, the granulation is continued until the size of the granule is bigger than 40 mesh, then dried until the water content is below 4%.
Both kinds of the above granules and magnesium stearate are mixed and then sprayed with peppermint ethanol solution and pressed into a tablet. After the weakly effervescent tablet disintegrates in neutral water, the pH of the suspension formed is more than 4.9, such that the formulation is in the form of an ODT.

### Embodiment 11

A weakly effervescent disintegrating tablet of an antihistamine such as levocetirizine, cetirizine, hydroxyzine, promethazine, fexofenadine, loratadine, desloratadine, diphenhydramine, terfenadine, chlorpheniramine, dexchlorpheniramine, clemastine, triprolidine, is manufactured using two processes of granulation:
1st granulation: an antihistamine selected from cetirizine, levocetirizine, terfenadine, hydroxyzine, promethazine, fexofenadine, loratadine, desloratadine, diphenhydramine, chlorpheniramine, clemastine, dexchlorpheniramine, triprolidine and calcium carbonate is mixed with about 5-20% of cross linked povidone of the weight of the tablet, 5-20% of cross linked carboxymethyl sodium cellulose of the weight of the tablet, and 5% of microcrystalline cellulose of the weight of the tablet, and sprayed with 8% PVPK30 in fluid bed, the granulation is continued until the size of the granule is bigger than 40 mesh, then dried until the water content is below 4%.
2nd granulation: one weak acid selected from the group consisting of malic acid, maleic acid, adipic acid, fumaric acid, tartaric acid, succinic acid and ascorbic acid, is mixed with about 5-20% of cross linked povidone of the weight of the tablet, 5-20% of cross linked carboxymethyl sodium cellulose of the weight of the tablet, 5% of microcrystalline cellulose of the weight of the tablet, and sprayed with 8% PVPK30 in a fluid bed, and the granule is dried until the water content is below 4%.
Both kinds of the above granules and magnesium stearate are mixed and then sprayed with peppermint ethanol solution and then pressed into a tablet. After the weakly effervescent tablet disintegrates in neutral water, the pH of the suspension formed is more than 4.9, such that the formulation is in the form of an ODT.

### Embodiment 12

A weakly effervescent disintegrating tablet of a serotonin reuptake inhibitor such as fluoxetine, paroxetine, sertraline, fluvoxamine, citalopram, escitalopram is manufactured using two processes of granulation:
1st granulation: a calcium carbonate is mixed with about 5-15% of cross linked povidone of the weight of the tablet, 5-15% of cross linked carboxymethyl sodium cellulose of the weight of the tablet, 5% of microcrystalline cellulose of the weight of the tablet, and sprayed with 8% PVPK30 in fluid bed, the granulation is continued until the size of the granule is bigger than 40 mesh, then it is dried until the water content is below 4%.
2nd granulation: an acid selected from malic acid, maleic acid, adipic acid, fumaric acid, tartaric acid, succinic acid and ascorbic acid is mixed with about 5-15% of cross linked povidone of the weight of the tablet, 5-15% of cross linked carboxymethyl sodium cellulose of the weight of the tablet, 5% of microcrystalline cellulose of the weight of the tablet, and one serotonin reuptake inhibitors selected from fluoxetine, paroxetine, sertraline, fluvoxamine, citalopram and escitalopram or a salt thereof, and sprayed with 8% PVPK30 in a fluid bed, the granule is then dried until the water content is below 4%.
Both kinds of the above granules and magnesium stearate are mixed and then sprayed with peppermint ethanol solution and then pressed into a tablet. After the weakly effervescent tablet disintegrates in the neutral water, the pH of the suspension formed is more than 4.9, such that the formulation is in the form of an ODT or a dispersible tablet.

### Embodiment 13

A weakly effervescent disintegrating tablet of a non-classical antidepressant such as aripiprazole, olanzapine, quetiapine, ziprasidone, clozapine, paliperidone is manufactured using two processes of granulation:
1st granulation: calcium carbonate is mixed with about 5-20% of cross linked povidone of the weight of the tablet, 5-20% of cross linked carboxymethyl sodium cellulose of the weight of the tablet, and 5% of microcrystalline cellulose of the weight of the tablet, and sprayed with 8% PVPK30 in a fluid bed, the granulation is continued until the size of the granule is bigger than 40 mesh, then dried until the water content is below 4%.
2nd granulation: one weak acid selected from malic acid, maleic acid, adipic acid, fumaric acid, tartaric acid, succinic acid and ascorbic acid is mixed with about 5-20% of cross linked povidone of the weight of the tablet, 5-20% of cross linked carboxymethyl sodium cellulose of the weight of the tablet, 5% of microcrystalline cellulose of the weight of the tablet and one non-classical antidepressant selected from aripiprazole, olanzapine, quetiapine, ziprasidone, clozapine, or paliperidone, and sprayed with 8% PVPK30 in fluid bed and the granule is then dried until the water content is below 4%.
Both kinds of the above granules and magnesium stearate are mixed and then sprayed with peppermint ethanol solution and then pressed into a tablet. After the weakly effervescent tablet disintegrates in the neutral water, the pH of the suspension formed is more than 4.9, such that the formulation is in the form of an ODT or a dispersible tablet.

### Embodiment 14

A weakly effervescent disintegrating tablet of an antifibrinolytic drug such as 6-aminocaproic acid (EACA), tranexamic acid, p-hydroxybenzylamine is manufactured using two processes of granulation:
1st granulation: sodium bicarbonate or potassium bicarbonate is mixed with about 5% of cross linked povidone of the weight of the tablet, 5% of cross linked carboxymethyl sodium cellulose of the weight of the tablet, 5% of microcrystalline cellulose of the weight of the tablet and one antifibrinolytic drug selected from 6-aminocaproic acid, tranexamic acid and p-hydroxybenzylamine, and sprayed with 8% PVPK30 in fluid bed, the granulation is continued until the size of the granule is bigger than 40 mesh, then dried until the water content is below 4%.
2nd granulation: An acid selected from monosodium malate, monosodium maleate, monosodium succinate, monosodium adipate, monosodium oxalate, monosodium citrate, ascorbic acid, monosodium tartrate, monopotassium malate, monopotassium maleate, monopotassium succinate, monopotassium adipate, monopotassium oxalate, monopotassium citrate and monopotassium tartrate, is mixed with about 5% of cross linked povidone of the weight of the tablet, 5% of cross linked carboxymethyl sodium cellulose of the weight of the tablet, 5% of microcrystalline cellulose of the weight of the tablet, and aspartame, and sprayed with 8% PVPK30 in fluid bed, the granulation is continued until the size of the granule is bigger than 40 mesh, then dried until the water content is below 4%.
Both kinds of the above granules and magnesium stearate are mixed and then sprayed with peppermint ethanol solution and then pressed into a tablet. After the weakly effervescent tablet disintegrates in neutral water, the pH of the suspension formed is more than 4.9, such that the composition is in the form of an ODT or a dispersible tablet.

### Embodiment 15

A weakly effervescent disintegrating tablet of a dihydropyridine calcium channel blocker such as nifedipine, felodipine, amlodipine, levamlodipine, benidipine, nitrendipine, nimodipine, lacidipine is manufactured using two processes of granulation:
1st granulation: a bicarbonate of sodium or potassium is mixed with about 5% to 20% of cross linked povidone of the weight of the tablet, 5% to 20% of cross linked carboxymethyl sodium cellulose of the weight of the tablet, 5% of microcrystalline cellulose of the weight of the tablet, and a dihydropyridine calcium channel blocker selected from nifedipine, felodipine, amlodipine, levamlodipine, benidipine, nitrendipine, nimodipine, lacidipine, and aspartame, and sprayed with 8% PVPK30 in a fluid bed, the granulation is continued until the size of the granule is bigger than 40 mesh, then dried until the water content is below 4%.
2nd granulation: one weak acid selected from monosodium malate, monosodium maleate, monosodium succinate, monosodium adipate, monosodium oxalate, monosodium citrate, monosodium tartrate, monopotassium malate, monopotassium maleate, monopotassium succinate, monopotassium adipate, monopotassium oxalate, monopotassium citrate, monopotassium tartrate, is mixed with about 5% to 20% of cross linked povidone of the weight of the tablet, 5% to 20% of cross linked carboxymethyl sodium cellulose of the weight of the tablet, 5% of microcrystalline cellulose of the weight of the tablet, and aspartame, and sprayed with 8% PVPK30 in a fluid bed, the granulation is continued until the size of the granule is bigger than 40 mesh, then dried until the water content is below 4%.
Both kinds of the above granules and magnesium stearate are mixed and then sprayed with peppermint ethanol solution and then pressed into a tablet. After the weakly effervescent tablet disintegrates in the neutral water, the pH of the suspension formed is more than 4.9, such that the composition is in the form of an ODT.

### EXAMPLES

The present invention will now be illustrated by the following examples of method of production of a cyclooxygenase-2 (COX-2) inhibitors composition and weakly effervescent disintegrating tablets, which are not to be construed as limiting the present invention in any manner and are only examples of the various formulations described herein.

### Example 1: A Comparison of the Composition of Celecoxib Tablet Containing Malic Acid Against CELEBREX 200 mg Capsule (Original Producer) is Shown in Table 3

TABLE 3
Composition of Lot 210607 with
CELEBREX Capsule lot 00024470

| | CELEBREX | |
|---|---|---|
| | Capsule lot | Lot |
| | 00024470 | 210607 |
| Celecoxib | 200 | 200 |
| Lactose | NO | 100 |
| SDS | NO | 10 |
| Cross linked povidone | NO | 50 |
| Cross linked sodium carboxymethylcellulose | YES | 100 |
| PVPK30 | YES | 7.9 |
| Magnesium stearate | YES | 4.7 |
| Content % | 99.3 | 99 |
| Dissolution at pH 12 of 1% sodium dodecyl | 96.5 | 29.9 |
| sulfate/sodium phosphate buffer solution under | | |
| 50 RPM at 60 minutes after dissolution test - % | | |

Celecoxib was dissolved in ethanol and mixed with lactose and SDS, the mixture was dried in oven and then smashed into fine powder and was granulated with other excipients listed in above table in wet granulator. The tablet made was tested for dissolution and the result was poorer than CELEBREX

### Example 2: A Comparison of the Composition of Celecoxib Tablet Containing Malic Acid Against CELEBREX 200 mg Capsule (Original Producer) in Shown in Table 4

TABLE 4
Comparison of composition of Lot 220208, Lot 220221,
Lot 20307 with CELEBREX Capsule lot 00024470

| | CELEBREX | | | |
|---|---|---|---|---|
| | Capsule lot | Lot | Lot | Lot |
| | 00024470 | 220208 | 220221 | 20307 |
| Celecoxib | 200 | 200 | 200 | 200 |
| Malic acid | NO | 200 | 300 | 300 |
| Microcrystalline cellulose | NO | 80 | 80 | 120 |
| Cross linked povidone | NO | 200 | 150 | 120 |
| Cross linked sodium | YES | | 150 | 120 |
| carboxymethylcellulose | | | | |
| SDS | YES | | | 30 |
| PVPK30 | YES | 22.9 | 29.2 | 33.3 |
| CaCO₃ | NO | 100 | 75 | 75 |
| Magnesium stearate | YES | 8.1 | 9.9 | 9.4 |
| Silicon dioxide | NO | 8.1 | 9.9 | 9.4 |
| Friability % | NA | 0.4 | 0.57 | 0.1 |
| Content % | 99.3 | 100.5 | 97.6 | 97.3 |
| Time of disintegration | | 100-135 | 132-140 | 122-155 |
| (seconds) | | | | |
| Dissolution at Ph 6.8 of | 61.2 | 57.1 | 59.1 | 62.2 |
| 0.25% sodium dodecyl | | | | |
| sulfate/Sodium Phosphate | | | | |
| Buffer solution under 50 RPM | | | | |
| at 60 minutes after dissolution | | | | |
| test - % | | | | |
| Dissolution at pH 6.8 of 1% | 88.3 | 79.6 | 92.7 | 95.1 |
| sodium dodecyl sulfate/ | | | | |
| sodium phosphate buffer | | | | |
| solution under 50 RPM at 60 | | | | |
| minutes after dissolution | | | | |
| test - % | | | | |

The celecoxib and malic acid was dissolved in ethanol, the mixture was heated in an oven, then the powder made was put in a granulator with other excipients listed above with PVPK30. The granules were obtained after passing through the mesh and was heated in an oven until the water content was below 4%. The granules obtained were mixed with magnesium stearate and compressed into a tablet. It was found that the amount of organic acid determines the rate of dissolution of celecoxib of the composition.

### Example 3: A Composition of Celecoxib Tablet with Malic Acid

Celecoxib with malic acid: 2250 g of cross linked carboxymethylcellulose sodium and 2250 g of cross linked povidone were mixed together in fluid bed. 2400 g of celecoxib and 4800 g of malic acid was dissolved in 10 litres of ethanol which was sprayed into the fluid bed, the mixture was dried, granules were made when a small amount of povidone K30 aqueous solution (300 g) was sprayed into the fluid bed. Total mixing: granules and magnesium stearate (9 g) were mixed together, and then compressed into tablets.

### Testing Result:

Hardness/pressure: 2.7-4.6 kg, tablet weight: 820 mg

Dissolution of celecoxib was 122 mg or 76.25% in 1000 mL of 0.25% sodium dodecyl sulfate/pH 7 sodium phosphate buffer solution under 50 RPM at 120 minutes after dissolution test. The whole granulation process may also be made by granulator.

The other compositions of celecoxib with different proportions of the acid selected from malic acid, maleic acid, tartaric acid, succinic acid, ascorbic acid, and citric acid, and cross linked carboxymethyl cellulose sodium, cross linked povidone is shown in table 5.

TABLE 5
Comparison of composition of celecoxib tablets with different proportion of the acids with CELEBREX

| | 1 | 2 | 3 | 4 | 5 | 6 | 7 | 8 | CELEBREX |
|---|---|---|---|---|---|---|---|---|---|
| Formula No. | mg | | | | | | | | |
| Celecoxib | 160 | 160 | 150 | 70 | 160 | 160 | 160 | 150 | 200 |
| Cross linked povidone | 150 | 150 | 150 | 70 | 320 | 150 | 150 | | |
| Sodium cross linked | 150 | 150 | 150 | 75 | | 150 | 150 | 280 | |
| carboxymethylcellulose | | | | | | | | | |
| Malic acid | 320 | 480 | 750 | 375 | | | | | |
| Maleic acid | | | | | | | 640 | | |
| Succinic acid | | | | | 320 | | | | |
| Tartaric acid | | | | | | | | 420 | |
| Ascorbic acid | | | | | | 480 | | | |
| Sodium dodecyl sulfate | 20 | 20 | 20 | 10 | 20 | 20 | 20 | 16 | |
| PVPK30 | 20 | 20 | 20 | 10 | 20 | 20 | 20 | 20 | |
| Calcium carbonate | | | | 15 | | | | | |
| Dissolution of celecoxib | 122/ | 27/ | 127/ | 127/ | 109/ | 117/ | 125/ | 122/ | 124/ |
| (mg) and rate of | 160 = | 160 = | 150 = | 140 = | 160 = | 160 = | 160 = | 150 = | 200 = |
| dissolution in 1000 mL of | 76% | 79.3% | 84.6% | 90.7% | 68.1% | 73% | 78% | 81.3% | 62% |
| 0.25% sodium dodecyl | | | | (2 | | | | | |
| sulfate/pH 7 sodium | | | | tablets | | | | | |
| phosphate buffer | | | | were | | | | | |
| solution under 50 RPM | | | | tested) | | | | | |
| at 120 minutes after | | | | | | | | | |
| dissolution test | | | | | | | | | |
| Dissociation of | | | | 98.3% | | | | | |
| calcium ions % 30 | | | | | | | | | |
| minutes after test | | | | | | | | | |

The dissociation of 200 mg celecoxib capsules of CELEBREX is 124 mg or 62% in 1000 mL of 0.25% sodium dodecyl sulfate/pH 7 sodium phosphate buffer solution under 50 RPM at 120 minutes after dissolution test.

The invention of the formulation of 150-160 mg tablets had similar dissolution amounts of 124-127 mg but had different dissolving rate of 76% to 84.6%. For formulation 1 to 3, the higher the amount of malic acid, the higher the dissolution of celecoxib. For formulation 5, the dissolution of celecoxib is lower than formulation 1, this is because the excipient of expander is cross linked povidone only. It seems that both cross linked povidone and sodium cross linked carboxymethyl cellulose is better than a single excipient. The proportion of organic acid to celecoxib is from 2:1 to 5:1, and the dissociation of celecoxib is proportional to the amount of organic acid in the composition.

### Example 4: A Weakly Effervescent Disintegrating Tablet of Calcium Carbonate

TABLE 6
Granules 1 of a weakly effervescent disintegrating
tablet of calcium carbonate in Example 4

| | A tablet (mg) | For 46700 tab.s (kg) |
|---|---|---|
| Granules 1 | | |
| Calcium carbonate | 150 | 7 |
| Microcrystalline | 30 | 1.4 |
| cellulose | | |
| Cross linked povidone | 20 | 0.934 |
| PVPK30 | 15 | 0.7 |
| Granules 2 | | |
| Malic acid | 100 | 4.67 |
| Aspartame | 1.0 | 0.0467 |
| Microcrystalline | 30 | 1.4 |
| cellulose | | |
| PVPK30 | 15 | 0.7 |
| Magnesium stearate | 3.52 | 0.164 |
| Peppermint | 0.864 | 0.04 |
| Ethanol | 2.6 | 0.121 |

The manufacture had two granulation processes:

Granulation 1: 7 kg of calcium carbonate, 1.4 kg of microcrystalline cellulose and 0.934 kg of cross linked povidone was put in a fluid bed. Then 8% of PVP K30 was sprayed into the fluid bed. The spraying was stopped when the size reached 30 mesh and the heating was stopped when the water content was less than 4%.

Granulation 2: 4.67 kg of malic acid 46.7 g of aspartame and 1.4 kg of microcrystalline cellulose was put into a fluid bed. Then 8% of PVP K30 was sprayed into the fluid bed. The spraying was stopped when the size reached 30 mesh and the heating was stopped when the water content was less than 4%.

Each of granules 1 and 2 were mixed with 0.164 kg of magnesium stearate, and sprayed with peppermint/ethanol solution, and then compressed into tablets.

The time of disintegration was 130-155 seconds, the amount of calcium ions of each tablet was 61 mg.

The friability was 0.4%. After the invention of weakly effervescent tablet disintegrated in the neutral water, the pH of the suspension formed was 5.91, such that the composition was a dispersible tablet.

Different molar ratios of malic acid to calcium carbonate (100 mg CaCO₃) in 900 mL of neutral pure water at 37° C. and 75 RPM at 30 minutes after dissolution test as shown in table 7.

TABLE 7
Dissolution test of Ca ions (%) of given
malic acid: CaCOs molar ratios

| | Malic acid:CaCO₃ molar ratio | | | | | |
|---|---|---|---|---|---|---|
| | 0.15:1 | 0.2:1 | 0.35:1 | 0.75:1 | 1:1 | 1.5:1 |
| Dissociation of | 24.0 | 31.0 | 45.2 | 66.6 | 74.7 | 94.2 |
| Ca ions % pH | 7.44 | 7.36 | 6.92 | 6.44 | 5.59 | 4.26 |

The example show the greater the amount of acid added, the higher the dissociation of calcium ion, the lower the amount of calcium carbonate needed to be administered to patients.

### Example 5: Comparison of Calcium Carbonate Tablet with Calcium Carbonate/Vitamin C and CaC₃/Vitamin D₃

TABLE 8
Comparison of Lot 0422, Lot 0312 with CaCO₃/Vit. D₃ (Caltrate)

| | | | CaCO₃/ |
|---|---|---|---|
| | Lot | Lot | Vit. D₃ |
| | 0422/mg | 0312/mg | (Caltrate)/mg |
| Granules 1 | A tablet | | |
| Calcium carbonate | 150 | 100 | 1500 |
| Microcrystalline cellulose | 30 | 20 | Unknown |
| Cross linked povidone | 0 | 25 | unknown |
| Menthol | 0.576 | | unknown |
| Granules 2 | | | |
| Malic acid | 120 | | unknown |
| Cross linked Povidone | | 25 | unknown |
| Ascorbic acid | | 175 | unknown |
| Aspartame | 1.05 | 1.14 | |
| Microcrystalline cellulose | 30 | 20 | |
| Menthol | | 0.576 | |
| PVPK30 | 15 | 12 | |
| Magnesium stearate | 3.52 | 3.8 | |
| Assay of Ca | 69 | 40.6 | 600 |
| Acute tablet weight | 460 | 382 | 1800 |
| Time of disintegration(s) | 170-180 | 75-90 | 250 |
| pH | 5.99 | 6.32 | 9.83 |
| Dissolving calcium ions after | 58.9% | 42% | 1.11% |
| 30 minutes in pH 7 water | | | |

As shown in table 8, this example shows that the addition of a small amount of expander could decrease the time of disintegration. This example also shows that the addition of acid in the composition may increase the dissolution of calcium ions in pH 7 pure water. This example also shows that the malic acid is stronger than the ascorbic acid.

### Example 6: A Weakly Effervescent Disintegrating Tablet of Calcium Carbonate and Vitamin C

TABLE 9
Manufacturing of a weakly effervescent tablet of calcium
carbonate and vitamin C in one tablet and For 70000 tab.s

| | A tablet (mg) | For 70000 tab.s (kg) |
|---|---|---|
| Granules 1 | | |
| Calcium carbonate | 100 | 7 |
| Microcrystalline cellulose | 20 | 1.4 |
| Cross linked povidone | 25 | 1.75 |
| PVPK30 | 12 | 0.84 |
| Granules 2 | | |
| Ascorbic acid | 175 | 12.25 |
| Aspartame | 1.14 | 0.08 |
| Microcrystalline cellulose | 30 | 2.1 |
| Cross linked povidone | 25 | 1.75 |
| PVPK30 | 15 | 1.05 |
| Magnesium stearate | 3.8 | 0.266 |
| Peppermint | 0.576 | 0.04 |

Granulation 1: 7 kg of calcium carbonate, 1.4 kg of microcrystalline cellulose and 1.75 kg of cross linked povidone was placed in a fluid bed. Then 8% of PVPK30 was sprayed into the fluid bed. The spraying was stopped when the granules were bigger than 40 mesh, and the heating was stopped when the water content was less than 4%.

Granulation 2: 12.25 kg of ascorbic acid, 80 g of aspartame and 2.1 kg of microcrystalline cellulose was placed into a fluid bed. Then 8% of PVPK30 was sprayed into the fluid bed. The spraying was stopped when the water content was less than 4%.

Each of granules 1 and 2 were mixed with 0.266 kg of magnesium stearate, and sprayed with peppermint/ethanol solution, then compressed into tablets.

The time of disintegration was 75-90 seconds. The amount of calcium ions of each tablet was 40.6 mg.

The friability was 0.31%. After the weakly effervescent tablet disintegrated in the neutral water, the pH of the suspension formed was 6.32, such that the composition was a dispersible tablet.

### Example 7: A Weakly Effervescent Disintegrating Tablet of Calcium Carbonate, Vitamin C and Glucosamine HCl

TABLE 10
Manufacturing of a weakly effervescent tablet of calcium carbonate,
vitamin C and glucosamine HCl in one tablet and for 20000 tab.s

| | Tablet/mg | For 20000 tabs (kg) |
|---|---|---|
| Granules 1 | | |
| Glucosamine HCl | 375 | 7.5 |
| Ascorbic acid | 125 | 2.5 |
| Cross linked povidone | 40 | 0.8 |
| Microcrystalline cellulose | 50 | 1 |
| PVPK30 | 12 | 0.24 |
| Granules 2 | | |
| Cross linked povidone | 10 | 0.2 |
| Calcium carbonate | 75 | 1.5 |
| Aspartame | 2 | 0.04 |
| Microcrystalline cellulose | 10 | 0.2 |
| PVPK30 | 2 | 0.04 |
| Lemon yellow mg | 0.07 | 0.0014 |
| Magnesium stearate | 7 | 0.14 |
| Peppermint | 0.8 | 0.016 |
| Ethanol | 2.4 | 0.048 |

Granulation 1: 7.5 kg of glucosamine HCl, 2.5 kg of ascorbic acid, 1.0 kg of microcrystalline cellulose and 0.8 kg of cross linked povidone was placed in a fluid bed. Then 8% of PVPK30 was sprayed into the fluid bed. The spraying was stopped when the size reached 30 mesh, and the heating was stopped when the water content was less than 4%.

Granulation 2: 1.5 kg of calcium carbonate, 0.2 kg of microcrystalline cellulose, 0.2 kg of cross linked povidone, and 40 g of aspartame was placed into a wet granulator. Then 8% of PVPK30 was sprayed into the machine. The wet granules was passed through the size of 30 mesh and it was heated in an oven for 6-8 hours at 65° C. until the water content was less than 4%.

Each of granules 1 and 2 were mixed with 0.14 kg of magnesium stearate, and sprayed with peppermint/ethanol solution, then compressed into tablets. The time of disintegration was 90-120 seconds.

The friability was 0.31%. After the weakly effervescent tablet disintegrated in the neutral water, the pH of the suspension formed was 6.32, such that the composition was a dispersible tablet.

### Example 8: A Weakly Effervescent Disintegrating Tablet of Vitamin C

Granulation 1: 0.7 kg of sodium bicarbonate, 0.7 kg of cross linked povidone, and 0.2 kg of microcrystalline cellulose, was placed into a wet granulator. Then 8% of PVPK30 with 0.6 g of lemon yellow powder was sprayed into the machine. The wet granules were passed through a size 30 mesh sieve, and heated in an oven for 6-8 hours at 65° C. until the water content was less than 4%.

Granulation 2: 4 kg of ascorbic acid, 0.4 kg of microcrystalline cellulose and 0.4 kg of cross linked povidone, 40 g of aspartame was put in a fluid bed. Then 8% of PVP K30 was sprayed into the fluid bed. The spraying was stopped when the size reached 30 mesh and the heating was stopped when the water content was less than 4%.

Each of granules 1 and 2 were mixed with 0.07 kg of magnesium stearate, and sprayed with peppermint/ethanol solution and then compressed into tablets.

The time of disintegration was 45-55 seconds. The friability was 0.41%. After the weakly effervescent tablet disintegrated in neutral pure water, the pH of the suspension formed was 6.02, such that the composition was an oral dispersible tablet (ODT).

### Example 9: A Weakly Effervescent Tablet of Multivitamins

TABLE 11
Manufacturing of a weakly effervescent tablet
of multivitamins in one tablet and 50000 tab.s

| | mg/tab. | g (50000 tab) |
|---|---|---|
| Fluid bed granulation (1) | | |
| Calcium carbonate | 150 | 7500 |
| Cross linked povidone | 30 | 1500 |
| Microcrystalline cellulose | 20 | 1000 |
| Lemon yellow powder | 0.04 | 2 |
| Granulation (2)/tab. | | |
| Folic acid | 1 | 50 |
| Ascorbic acid | 150 | 7500 |
| Vitamin B1 | 0.5 | 25 |
| Vitamin B2 | 0.5 | 25 |
| Vitamin B6 | 0.5 | 25 |
| Vitamin B12 | 0.005 | 0.25 |
| Vitamin A | 0.16 | 8 |
| Vitamin E | 5.00 | 250 |
| Vitamin K | 0.015 | 0.75 |
| Aspartame | 0.98 | 49 |
| Cross povidone | 20 | 1000 |
| Microcrystalline cellulose | 20 | 1000 |
| Vitamin D3 | 0.01 | 0.5 |
| Magnesium stearate | 3.2 | 160 |

Granulation 1: 7.5 kg of calcium carbonate, 1.5 kg of cross linked povidone, and 1.0 kg of microcrystalline cellulose, was placed into a fluid bed. Then 8% of PVPK30 with 2 g of lemon yellow powder was sprayed into the machine. The granulation was stopped when the size of granule reached 30 mesh, and heated until the water content was less than 4%.

Granulation 2: 7.5 kg of ascorbic acid, 1.0 kg of microcrystalline cellulose and 1.0 kg of cross linked povidone, and 49 g of aspartame was placed in a fluid bed. Then 800 mL of solution containing 50 g of folic acid, 25 g of vitamin B1, 25 g of vitamin B2, 25 g of vitamin B6, 0.25 g of vitamin B12 was sprayed into the fluid bed. Then 1000 mL of 95% ethanol containing 8 g of vitamin A, 250 g of vitamin E, 0.75 of g of vitamin K and 0.5 g of vitamin D3 was sprayed into the fluid bed containing the ascorbic acid. Then 8% of PVPK30 was sprayed into the fluid bed. The spraying was stopped when the size reached 30 mesh, and the heating was stopped when the water content was less than 4%.

Each of granules 1 and 2 were mixed with 0.16 kg of magnesium stearate, and sprayed with peppermint/ethanol solution, then compressed into tablets.

The time of disintegration was about 91-100 seconds. The friability was 0.41%. After the weakly effervescent tablet disintegrated in the neutral water, the pH of the suspension formed was 6.0, such that the composition was a dispersible tablet.

### Example 10: A Weakly Effervescent Disintegrating Tablet of Nifedipine (10 Ma×12000 Tablets)

1. Nifedipine (200 g) was dissolved in 4000 g of 96% ethanol. Aspartame (64 g) and cross linked povidone (1200 g) was added into the above solution and stirred evenly. The mixture was dried in the oven until the alcohol was substantially volatilized. The dry powder was then crushed through an 80 mesh sieve to obtain nifedipine mixture (1336 g).
2. Menthol (21.6 g) was added into 26 g of 96% ethanol.
3. Nifedipine mixture (878.4 g) from step 1 and sodium hydrogen tartrate (180 g) were placed into a wet granulation machine to mix evenly, then povidone K30 aqueous solution (559 g) was added. The contents of the machine were stirred and sieved through a 30 mesh sieve. The resultant granules were dried for about 4 hours until the water content was 3.24%, then menthol ethanol solution (47.6 g) was sprayed into the granules which were then heated for about 1-2 hours.
4. Total mixing: granules (1008 g), sodium bicarbonate (77.95 g) and magnesium stearate (10 g) were mixed together and then compressed into the tablet.

### Testing Result:

Hardness/pressure: 1.0-1.5 kg
Tablet weight: 107 mg
Disintegration time: CHP 39-46 s; USP 20-36 s
Brittleness: 0.21%

After the weakly effervescent tablet disintegrated in neutral water, the pH of the suspension formed was 7.45, such that the composition was an ODT.

### Example 11: A Weakly Effervescent Disintegrating Tablet of Celecoxib (200 mg)

1. Povidone K30 (32 g) was dissolved into pure water (600 g) and stirred evenly.
2. Menthol (50 g) was added into of 96% ethanol (65 g) and stirred well.
3. Celecoxib (800 g), aspartame (70 g), microcrystalline cellulose (100 g) and monosodium hydrogen tartrate (310 g) were added to the wet granulating machine to mix evenly, then povidone K30 aqueous solution (300 g) was added to the granulator which stirred the materials for 100 sec. The material was screened through a 30 mesh sieve, and dried until the water content was 3.4%. Menthol ethanol solution (115 g) was then sprayed into the granules. The granules were sieved through a 30 mesh sieve.
4. Total mixing: granules (1200 g), sodium bicarbonate (170 g), sodium cross linked carboxymethylcellulose (75 g), silicon dioxide (14 g) and magnesium stearate (14 g) were mixed together, and then compressed into tablets.

### Testing Result:

Hardness/pressure: 2.7-4.6 kg
Tablet weight: 360 mg
Disintegration time: CHP 48-55 s; USP 26-35 s
Brittleness: 0.72%

After the weakly effervescent celecoxib tablet disintegrated in the neutral water, the pH of the suspension formed was 6.06, such that the composition was an ODT.

### Example 12: A Weakly Effervescent Disintegrating Tablet of Irbesartan (150 mg)

1. Povidone K30 (24 g) was dissolved in pure water (456 g) with even stirring.
2. Menthol (40 g) was added into 96% ethanol (48 g) and stirred well.
3. Irbesartan (600 g), aspartame (52 g), microcrystalline cellulose (120 g) and monosodium hydrogen tartrate (308 g) were placed into the wet granulating machine to mix evenly, then povidone K30 aqueous solution (300 g) was added to the granulator, within which the materials were stirred for 100 s. The material was screened for 30 mesh, and dried until the water content was 3.6%. Then menthol ethanol solution (88 g) was sprayed into the granules. The granules were sieved through a 30 mesh screen.
4. Total mixing: granules (1058 g), sodium bicarbonate (170 g), sodium cross linked carboxymethyl cellulose (61.8 g), silicon dioxide (12.8 g) and magnesium stearate (12.8 g) were mixed, and then compressed into tablets.

### Testing Result:

Hardness/pressure: 2.5-3.6 kg
Tablet weight: 360 mg
Disintegration time: CHP 53-60 s; USP 32-43 s
Brittleness: 0.52%

After the weakly effervescent irbesartan tablet disintegrated in neutral water, the pH of the suspension formed was 5.9, such that the composition was an ODT

### Example 13: Effect of Different Ratio of Ascorbic Acid on Calcium Dissolution in 900 mL of pH 7 Pure Water

(a) Calcium carbonate (25 mg)/ascorbic acid (25 mg) tablet

TABLE 12
Dissolution test of calcium carbonate
(25 mg)/ascorbic acid (25 mg) tablet

| Time mins. | 5 | 10 | 20 | 30 |
|---|---|---|---|---|
| Dissolution % | 33.0 | 34.42 | 39.6 | 44.0 |

(b) Calcium carbonate (25 mg)/ascorbic acid (50 mg) tablet

TABLE 13
Dissolution test of calcium carbonate
(25 mg)/ascorbic acid (50 mg) tablet

| Time mins. | 5 | 10 | 20 | 30 |
|---|---|---|---|---|
| Dissolution % | 43.54 | 48.46 | 51.48 | 52.46 |

(c) Calcium carbonate (25 mg)/ascorbic acid_(75 mg) tablet

TABLE 14
Dissolution test of calcium carbonate
(25 mg)/ascorbic acid (75 mg) tablet

| Time mins. | 5 | 10 | 20 | 30 |
|---|---|---|---|---|
| Dissolution % | 58.7 | 59.4 | 60.35 | 65.28 |

(d) Calcium carbonate (25 mg)/ascorbic acid (75 mg) tablet+25 mg ascorbic acid in the vessel

TABLE 15
Dissolution test of calcium carbonate (25 mg)/ascorbic
acid (75 mg) tablet + 25 mg ascorbic acid

| Time mins. | 5 | 10 | 20 | 30 | 60 |
|---|---|---|---|---|---|
| Dissolution % | 56.1 | 58.7 | 61.4 | 63.6 | 67.1 |

The above examples show that dissolution of the calcium increased proportionally with the addition of ascorbic acid, but after the ratio of the ascorbic acid to calcium carbonate was above 3:1, the dissolution of calcium was not further increased. Accordingly, a preferred ratio of ascorbic acid to calcium carbonate in the composition is from 1:1 to 3:1, preferably 3:1.

### Example 14: Different Molar Ratio of Citric Acid to Calcium Carbonate on Calcium Ion Dissolution in 900 mL of Pure Neutral Water at 30 Minutes after Stirring at 37° C.

Calcium carbonate (100 mg)/192 mg citric acid tablet (1:1)
Calcium carbonate (100 mg)/172.8 mg citric acid tablet (1:0.9)
Calcium carbonate (100 mg)/154 mg citric acid tablet (1:0.8)
Calcium carbonate (100 mg)/134 mg citric acid tablet (1:0.7)

TABLE 16
dissolution test of calcium ions in different molar
ratios of citric acid to calcium carbonate

| | CaCO₃:citric acid molar ratio | | | | | |
|---|---|---|---|---|---|---|
| | 1:1 | 1:0.9 | 1:0.8 | 1:0.7 | 1:0.6 | 1:0.25 |
| Dissolution % at 30 | 85.91 | 81 | 78 | 74 | 72 | 45.0 |
| minutes pH of the | 4.72 | 4.86 | 5.22 | 5.4 | 5.9 | 7.03 |
| resultant solution | | | | | | |

Since the citric acid is the ternary acid, it is stronger than binary acid. The molar ratio of citric acid to calcium carbonate should be less than 0.8:1 to make the oral disintegration tablet safe.

### Example 15: A Composition of 135 mg Celecoxib Pellet with 135 mg HPMC E5

2700 gram of celecoxib with 2700 g of HPMC E5 was dissolved in 25.6 litres of 95% ethanol, which was sprayed into a fluid bed containing 2800 g of sugar pellet of 0.5-0.7 mm size. Mixing: pellet and magnesium stearate were mixed together, and then filled into capsules.

### Testing Result:

Dissolution of celecoxib was 112 mg or 83% in 1000 mL of neutral pure water containing 2.5 g of sodium dodecyl sulfate under 50 RPM at 180 minutes after dissolution test.

### Example 16: Beagle Dog Bioavailability Study of 150 mg Celecoxib Tablet Against CELEBREX 200 mg Capsules

Bioavailability Study of celecoxib in dogs was studied in 4 healthy beagle dogs (male dogs weighing: A1 (13.6 kg), B1 (10.8 kg); female dogs weighing: A2 (10.7 kg), B2 (16.0 kg)).

A 150 mg celecoxib tablet of US NANO batch number: S220519 were studied in 4 beagle dogs against the control drug of 200 mg CELEBREX capsule manufactured by Pfizer Pharmaceuticals LLC, batch number: DN4886. (Manufacturer: Pfizer Pharmaceuticals LLC; Location: Road 689, Km. 1.9 Vega Baja, Puerto Rico 00693, CHINA Sub-packaging Factory: Pfizer Pharmaceutical Co., LTD; Address: No. 22 Daqing Road, Dalian Economic and Technological Development Zone).

TABLE 17
Components of a 150 mg celecoxib tablet
of US NANO batch number: S220519

| Components | mg/tablet |
|---|---|
| Celecoxib | 150 |
| Cross linked povidone | 186 |
| Sodium cross linked carboxymethylcellulose | 186 |
| Malic acid | 450 |
| HPMC E5 | 20.7 |
| Sodium dodecyl sulfate | 29 |
| PVPK30 | 20.7 |
| Dissolution of celecoxib (mg) and rate of dissolution | 143.8 mg (95.9%) |
| in 1000 mL of 0.25% sodium dodecyl sulfate/pH 7 | |
| sodium phosphate buffer solution under 50 RPM at | |
| 120 minutes after dissolution test | |

Each of 2 beagle dogs were fed with 1 capsule of CELEBREX (200 mg) on 12 Oct. 2021 and each of another 2 beagle dogs were fed with 1 capsule of CELEBREX (200 mg) on 19 Oct. 2021. Serial blood samples (˜4 mL) were collected from individual animals at pre-dose, 0.5, 1, 2, 3, 4, 6, 8, 12, 24 h after dosing via lateral saphenous venipuncture using heparin as anticoagulant. Plasma was collected after centrifugation of the samples and was extracted via acetonitrile precipitation and centrifuged. The plasma concentration of celecoxib was determined by UPLC-MS using the standard plasma curve method.

Each of 4 beagle dogs were fed with 1 tablets of celecoxib 150 mg tablet of the invention of US NANO on 25 May 2022. Serial blood samples (˜4 mL) were collected from individual animals at pre-dose, 0.5, 1, 2, 3, 4, 6, 8, 12, 24 h after dosing via lateral saphenous venipuncture using heparin as anticoagulant. Plasma was collected after centrifugation of the samples and was extracted via acetonitrile precipitation and centrifuged. The plasma concentration of celecoxib was determined by UPLC-MS using the standard plasma curve method.

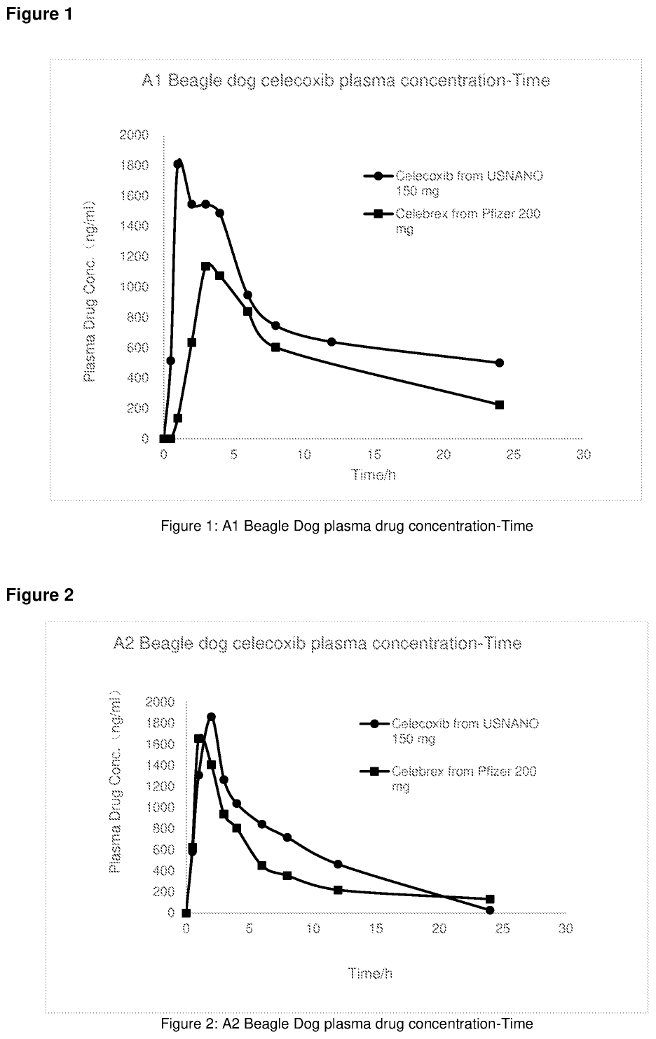

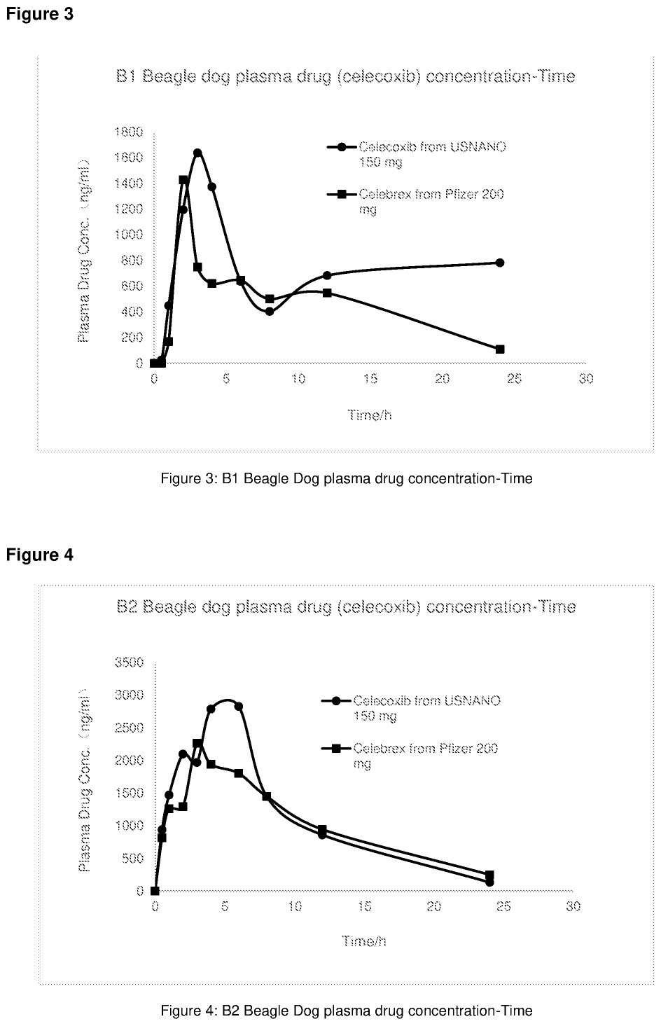

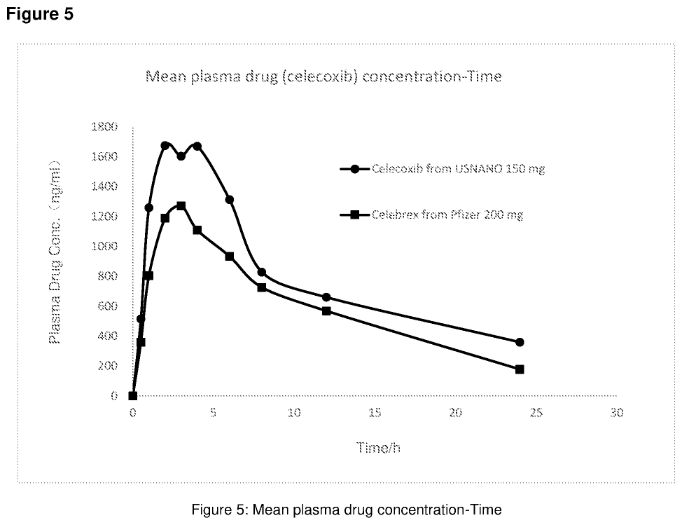

The washout period is more than 6 months between 2 drug administrations in the dogs. The supernatant was injected directly into UHPLC-MS for analysis, resulting data were used to calculate pharmacokinetic parameters presented in table 18, and the average testing results of blood plasma concentration-time are listed as FIGS. 1 to 5.

TABLE 18
Average Pharmacokinetic Parameters of CELEBREX
from Pfizer and Celecoxib from US NANO

| | CELEBREX | Celecoxib |
|---|---|---|
| | from Pfizer | from US NANO |
| Parameters | (n = 4) | (n = 4) |
| Dosage Form | capsule | tablet |
| Batch No. | DN4886 | S220519 |
| Specification | 200 mg | 150 mg |
| Tₘₐₓ (h) | 2 (1.0, 3.0) | 3 (1.0, 6.0) |
| Cₘₐₓ (ng/mL) | 1619.69 ± 418.47 | 2033.54 ± 465.36 |
| AUC0→t (h*ng/mL) | 14460.15 ± 6055.03 | 19544.00 ± 5012.31 |
| AUC0→∞ (h*ng/mL) | 21700.62 ± 8152.05 | 30156.39 ± 13255.32 |
| ∧z (h−1) | 0.0307 ± 0.0106 | 0.0465 ± 0.0209 |
| t½ (h) | 26.57 ± 12.06 | 20.37 ± 12.43 |

In the preceding specification, the invention has been described with reference to specific exemplary embodiments and examples thereof. It will, however, be evident that various modifications and changes may be made thereto without departing from the broader spirit and scope of the invention as set forth in the claims that follow. Additionally, it is contemplated that such composition may be utilized at additional sites not specifically mentioned herein. Such obvious modifications are considered to be within the scope of the appended claims. The specification is accordingly to be regarded in an illustrative manner rather than a restrictive sense.

### FORMS OF THE INVENTION

1. A method of producing a composition comprising a cyclooxygenase-2 (COX-2) inhibitor selected from the group consisting of celecoxib and imrecoxib;
   one or more water soluble excipients selected from a group consisting of a HPMC (hydroxypropyl methylcellulose); and
   an organic acid selected from the group consisting of malic acid, maleic acid, succinic acid, tartaric acid, citric acid, ascorbic acid or a mixture thereof,
   the method comprising a step of dissolving the COX-2 inhibitor and the water soluble excipient in alcohol and mixing with an expander and filler,
   wherein the composition is in the form of a pellet, a capsule, granules or a tablet.
2. The method of form 1 wherein the cyclooxygenase-2 (COX-2) inhibitor is celecoxib, and wherein the composition further comprises cross linked povidone, cross linked carboxymethyl sodium cellulose, and magnesium stearate.
3. The method of form 1, wherein the cyclooxygenase-2 (COX-2) inhibitor is celecoxib, and wherein the composition further comprises cross linked povidone and cross linked carboxymethyl sodium cellulose, wherein the composition has a dissolution rate of more than 60% of celecoxib in 1000 mL of 0.25% sodium dodecyl sulfate in pH 7 sodium phosphate buffer solution when stirred under 50 RPM at 120 minutes.
4. The method of any one of forms 1 to 3, wherein the wherein the amount of organic acid is from 1 to 6 times the weight of celecoxib in the composition.
5. The method of any one of forms 1 to 4, wherein the cyclooxygenase-2 (COX-2) inhibitor is celecoxib and the organic acid is malic acid.
6. The method of any one of forms 1 to 4, wherein the cyclooxygenase-2 (COX-2) inhibitor is celecoxib and the organic acid is tartaric acid.
7. The method of any one of forms 1 to 4, wherein the cyclooxygenase-2 (COX-2) inhibitor is celecoxib and the organic acid is maleic acid.
8. The method of any one of forms 1 to 4, wherein the cyclooxygenase-2 (COX-2) inhibitor is celecoxib and the organic acid is succinic acid.
9. The method of any one of forms 1 to 4, wherein the cyclooxygenase-2 (COX-2) inhibitor is celecoxib and the organic acid is citric acid.
10. The method of any one of forms 1 to 4, wherein the cyclooxygenase-2 (COX-2) inhibitor is celecoxib and the organic acid is ascorbic acid.
11. The method of form 1 wherein the cyclooxygenase-2 (COX-2) inhibitor is imrecoxib, wherein the composition further comprises cross linked povidone, cross linked carboxymethyl sodium cellulose, and magnesium stearate, wherein the composition has a dissolution of imrecoxib of more than 60% in 1000 mL of 0.25% sodium dodecyl sulfate in pH 7 sodium phosphate buffer solution when stirred under 50 RPM at 120 minutes.
12. The method of form 1 wherein the cyclooxygenase-2 (COX-2) inhibitor is celecoxib, the organic acid is selected from the group consisting of maleic acid, succinic acid, tartaric acid, malic acid, citric acid and ascorbic acid; the composition further comprises cross linked povidone, cross linked carboxymethyl sodium cellulose, calcium carbonate, and magnesium stearate, and wherein the composition has a dissolution rate of celecoxib of more than 60% in 1000 mL of 0.25% sodium dodecyl sulfate in pH 7 sodium phosphate buffer solution when stirred under 50 RPM at 120 minutes.
13. The method of form 1 wherein the cyclooxygenase-2 (COX-2) inhibitor is imrecoxib, calcium carbonate, the organic acid is selected from the group consisting of maleic acid, succinic acid, tartaric acid, malic acid, citric acid and ascorbic acid; the composition further comprises cross linked povidone, cross linked carboxymethyl sodium cellulose and magnesium stearate.
14. The method of form 1 wherein the composition comprises celecoxib, hydroxypropyl methylcellulose and pellets consisting of sugar or mannitol such that the composition is in the form of a pellet or a capsule.
15. The method of form 1 wherein the composition comprises celecoxib, 1 to 2 parts of hydroxypropyl methylcellulose and 1 to 2 parts of pellets consisting of sugar or mannitol such that the composition is in the form of pellets or a capsule, and wherein the composition has a dissolution rate of celecoxib of more than 60% in 1000 mL of neutral pure water containing 2.5 g of sodium dodecyl sulfate when stirred under 50 RPM at 180 minutes.
16. The method of form 1 wherein the composition comprises imrecoxib, hydroxypropyl methylcellulose and pellets consisting of sugar or mannitol, wherein the composition is in the form of pellets or a capsule.
17. The method of form 1 wherein the composition comprises 1 part of imrecoxib, 1 to 2 parts of hydroxypropyl methylcellulose and 1 to 2 parts of pellets consisting of sugar or mannitol, wherein the composition is in the form of pellets or a capsule, and wherein the composition has a dissolution rate of imrecoxib of more than 60% in 1000 mL of neutral pure water containing 2.5 g of sodium dodecyl sulfate when stirred under 50 RPM at 180 minutes.
18. A weakly effervescent disintegration formulation comprising a weak acid-base pair, wherein the weak acid is selected from monosodium salt of a binary acid, monopotassium salt of a binary acid, monosodium salt of a ternary acid, ascorbic acid, a binary acid, or mixtures thereof; and the weak base is selected from calcium carbonate, sodium bicarbonate, potassium bicarbonate and lithium bicarbonate, and an API which is selected from the group consisting of calcium carbonate, glucosamine, acarbose, miglitol, dihydropyridine calcium channel blocker, nimodipine, triptans, antihistamines, sartans, 5-HT3 antagonist antiemetic drugs, antifibrinolytic drugs, non-classical antidepressants, NSAIDs, hormone contraceptives and anti-epileptics; wherein the formulation produces a pH of greater than 4.9 when disintegrated in neutral pure water.
19. The weakly effervescent disintegration formulation of form 18 comprising calcium carbonate, ascorbic acid, cross linked povidone, and magnesium stearate, wherein the ratio of the weight of ascorbic acid to the weight of calcium carbonate is in the range of 1:1 to 3:1, and wherein the composition is in the form of a capsule, a tablet, an oral disintegration tablet or a dispersible tablet, wherein the formulation produces a pH of greater than 4.9 when disintegrated in 200 mL of neutral pure water, and wherein the formulation has a dissolution of calcium ions in the range of 20% to 65% in 900 mL of neutral water when stirred at a speed of 75 RPM and measured at 30 min.
20. The weakly effervescent disintegration formulation of form 18 comprising calcium carbonate, an organic acid selected from the group consisting of tartaric acid, malic acid, maleic acid and succinic acid, and wherein the molar ratio of the acid to calcium carbonate is in the range of 0.15:1.0 to 1.0:1.0, and wherein the composition is in the form of granules, a capsule, a tablet or a dispersible tablet, and wherein the formulation produces a pH of greater than 4.9 when disintegrated in 200 mL of neutral pure water, and wherein the formulation has a dissolution of calcium ions in the range of 20% to 75% in 900 mL of neutral pure water when stirred at a speed of 75 RPM and measured at 30 min.
21. The weakly effervescent disintegration formulation of form 18 comprising calcium carbonate, citric acid, cross linked povidone, and magnesium stearate, wherein the molar ratio of citric acid to calcium carbonate is less than 0.8:1, wherein the formulation is in the form of granules, a capsule, a tablet or a dispersible tablet, and wherein the formulation produces a pH of greater than 4.9 when disintegrated in 200 mL of neutral water, and wherein the formulation has a dissolution of calcium ions in the range of 20% to 75% in 900 mL of neutral pure water when stirred at a speed of 75 RPM and measured at 30 min.
22. The weakly effervescent disintegration formulation of form 18 comprising calcium carbonate, maleic acid, cross linked povidone, and magnesium stearate, wherein the molar ratio of maleic acid to calcium carbonate is from 0.15:1 to 1:1, wherein the formulation is in the form of granules, a capsule, a tablet, an oral disintegration tablet or a dispersible tablet, wherein the formulation produces a pH of greater than 4.9 when disintegrated in 200 mL of neutral pure water, and wherein the formulation has a dissolution of calcium ions in the range of 20% to 75% in 900 mL of neutral pure water when stirred at a speed of 75 RPM and measured at 30 min.
23. The weakly effervescent disintegration formulation of form 18 comprising calcium carbonate, malic acid, cross linked povidone and magnesium stearate, wherein the molar ratio of malic acid to calcium carbonate is from 0.15:1 to 1:1, wherein the formulation is in the form of granules, a capsule, a tablet, an oral disintegration tablet or a dispersible tablet, wherein the formulation produces a pH of greater than 4.9 when disintegrated in 200 mL of neutral pure water, and wherein the formulation has a dissolution of calcium ions in the range of 20% to 75% in 900 mL of neutral pure water when stirred at a speed of 75 RPM and measured at 30 min.
24. The weakly effervescent disintegration formulation of form 18 comprising calcium carbonate, tartaric acid, cross linked povidone and magnesium stearate, wherein the molar ratio of tartaric acid to calcium carbonate is from 0.15:1 to 1:1, wherein the formulation is in the form of granules, a capsule, a tablet, a dispersible tablet or an oral disintegration tablet, wherein the formulation produces a pH of greater than 4.9 when disintegrated in 200 mL of neutral pure water, and wherein the formulation has a dissolution of calcium ions in the range of 20% to 75% in 900 mL of neutral pure water when stirred at a speed of 75 RPM and measured at 30 min.
25. The weakly effervescent disintegration formulation of form 18 comprising calcium carbonate, succinic acid, cross linked povidone and magnesium stearate, wherein the molar ratio of succinic acid to calcium carbonate is from 0.15:1 to 1:1, wherein the formulation produces a pH of greater than 4.9 when disintegrated in 200 mL of neutral pure water, and wherein the formulation has a dissolution of calcium ions in the range of 20% to 75% in 900 mL of neutral pure water when stirred at a speed of 75 RPM and measured at 30 min.
26. The weakly effervescent disintegration formulation of form 18 comprising calcium carbonate, glucosamine hydrochloride or its salt, cross linked povidone, and magnesium stearate, wherein the formulation is in the form of granules or a tablet, and wherein the formulation has a dissolution of calcium ions of about 35% to 55% in 900 mL of neutral pure water when stirred at a speed of 75 RPM and measured at 30 min; and wherein the pH of the suspension formed is from 6.5 to 7.5.
27. The weakly effervescent disintegration formulation of form 18 comprising sodium bicarbonate, glucosamine hydrochloride or its salt, cross linked povidone, and magnesium stearate, wherein the formulation is in the form of granules, a capsule, or a dispersible tablet, and wherein the formulation has a pH in the range of 6.5 to 7.5 after disintegration in 200 mL of neutral pure water.
28. The weakly effervescent disintegration formulation of form 18 comprising sodium bicarbonate, glucosamines sulfate, cross linked povidone, and magnesium stearate, wherein the formulation is in the form of granules, a capsule, or a dispersible tablet, and wherein the formulation has a pH in the range of 6.5 to 7.5 after disintegration in 200 mL of neutral pure water.
29. The weakly effervescent disintegration formulation of form 18 comprising acarbose, calcium carbonate, an acid selected from the group consisting of malic acid, maleic acid, tartaric acid, succinic acid, ascorbic acid and citric acid, and cross linked povidone, wherein the formulation is in the form of an oral disintegration tablet, and wherein the formulation produces a pH of more than 4.9 after disintegration in 200 mL of neutral pure water.
30. The weakly effervescent disintegration formulation of form 18 comprising miglitol, calcium carbonate, an acid selected from the group consisting of malic acid, maleic acid, tartaric acid, succinic acid, ascorbic acid and citric acid, and cross linked povidone, wherein the formulation is in the form of an oral disintegration tablet, and wherein the formulation produces a pH of more than 4.9 after disintegration in 200 mL of neutral pure water.
31. The weakly effervescent disintegration formulation of form 18 comprising megestrol, calcium carbonate, an acid selected from the group consisting of malic acid, maleic acid, tartaric acid, succinic acid, ascorbic acid and citric acid, and cross linked povidone, wherein the formulation is in the form of an oral disintegration tablet, and wherein the formulation produces a pH of more than 4.9 after disintegration in 200 mL of neutral pure water.
32. The weakly effervescent disintegration formulation of form 18 comprising progesterone, calcium carbonate, an acid selected from the group consisting of malic acid, maleic acid, tartaric acid, succinic acid, ascorbic acid and citric acid, and cross linked povidone, wherein the formulation is in the form of an oral disintegration tablet, and wherein the formulation produces a pH of more than 4.9 after disintegration in 200 mL of neutral pure water.
33. The weakly effervescent disintegration formulation of form 18 comprising a weak base selected from a group consisting of sodium bicarbonate and potassium bicarbonate, an acid selected from the group consisting of monosodium malate, monosodium maleate, monosodium succinate, monosodium citrate, and monosodium tartrate, cross linked povidone and nifedipine, wherein the formulation is in the form of an oral disintegration tablet, and wherein the formulation produces a pH of more than 4.9 after disintegration in 200 mL of neutral pure water.
34. The weakly effervescent disintegration formulation of form 18 comprising a weak base selected from a group consisting of sodium bicarbonate and potassium bicarbonate, an acid selected from the group consisting of monosodium malate, monosodium maleate, monosodium succinate, monosodium citrate, and monosodium tartrate, cross linked povidone and nimodipine, wherein the formulation is in the form of an oral disintegration tablet, and wherein the formulation produces a pH of more than 4.9 after disintegration in 200 mL of neutral pure water.
35. The weakly effervescent disintegration formulation of form 18 comprising a weak base selected from a group consisting of sodium bicarbonate and potassium bicarbonate, an acid selected from a group consisting of monosodium malate, monosodium maleate, monosodium succinate, monosodium citrate, and monosodium tartrate, cross linked povidone, cross linked carboxymethyl sodium cellulose, and a sartan API selected from losartan, candesartan, valsartan, telmisartan, fimasartan, irbesartan or a salt thereof; wherein the formulation is in the form of an oral disintegration tablet or a dispersible tablet, and wherein the formulation produces a pH of more than 4.9 after disintegration in 200 mL of neutral pure water.
36. The weakly effervescent disintegration formulation of form 18 comprising sodium bicarbonate, monosodium tartrate, cross linked carboxymethyl sodium cellulose and valsartan, wherein the formulation is in the form of an oral disintegration tablet, and wherein the formulation produces a pH of more than 4.9 after disintegration in 200 mL of neutral pure water.
37. The weakly effervescent disintegration formulation of form 18 comprising sodium bicarbonate, monosodium tartrate, cross linked carboxymethyl sodium cellulose and irbesartan, wherein the formulation is in the form of an oral disintegration tablet, and wherein the formulation produces a pH of more than 4.9 after disintegration in 200 mL of neutral pure water.
38. The weakly effervescent disintegration formulation of form 18 comprising a bicarbonate, an acid selected from a group consisting of monosodium malate, monosodium maleate, monosodium succinate, monosodium citrate, monosodium tartrate, monopotassium malate, monopotassium maleate, monopotassium succinate, and monopotassium tartrate, cross linked povidone and an antifibrinolytic drug selected from 6-aminocaproic acid or tranexamic acid, wherein the formulation produces a pH of more than 4.9 after disintegration in 200 mL of neutral pure water.
39. The weakly effervescent disintegration formulation of form 18 comprising sodium bicarbonate, monosodium tartrate, cross linked carboxymethyl sodium cellulose and tranexamic acid, wherein the formulation is in the form of a dispersible tablet, and wherein the formulation produces a pH of more than 4.9 after disintegration in 200 mL of neutral pure water.
40. The weakly effervescent disintegration formulation of form 18 comprising calcium carbonate, an organic acid selected from the group consisting of malic acid, maleic acid, succinic acid, tartaric acid, citric acid and ascorbic acid, cross linked povidone, and a serotonin reuptake inhibitor selected from fluoxetine, paroxetine, sertraline, fluvoxamine or a salt thereof; wherein the formulation is in the form of an oral disintegration tablet, and wherein the formulation produces a pH of more than 4.9 after disintegration in 200 mL of neutral pure water.
41. The weakly effervescent disintegration formulation of form 18 comprising calcium carbonate, malic acid, cross linked carboxymethyl sodium cellulose, and sertraline, wherein the formulation is in the form of an oral disintegration tablet, and wherein the formulation produces a pH of more than 4.9 after disintegration in 200 mL of neutral pure water.
42. The weakly effervescent disintegration formulation of form 18 comprising calcium carbonate, malic acid, cross linked carboxymethyl sodium cellulose, and fluvoxamine, wherein the formulation is in the form of an oral disintegration tablet, and wherein the formulation produces a pH of more than 4.9 after disintegration in 200 mL of neutral pure water.
43. The weakly effervescent disintegration formulation of form 18 comprising calcium carbonate, an organic acid selected from the group consisting of maleic acid, malic acid, succinic acid, tartaric acid, ascorbic acid and citric acid, cross linked povidone, and fexofenadine, wherein the formulation is in the form of an oral disintegration tablet, and wherein the formulation produces a pH of more than 4.9 after disintegration in 200 mL of neutral pure water.
44. The weakly effervescent disintegration formulation of form 18 comprising calcium carbonate, an organic acid selected from the group consisting of malic acid, maleic acid, succinic acid, tartaric acid, ascorbic acid and citric acid, cross linked povidone, and a non-classical antidepressant selected from quetiapine or ziprasidone, wherein the formulation is in the form of an oral disintegration tablet, and wherein the formulation produces a pH of more than 4.9 after disintegration in 200 mL of neutral pure water.
45. The weakly effervescent disintegration formulation of form 18 comprising calcium carbonate, malic acid, cross linked povidone and quetiapine, wherein the formulation is in the form of an oral disintegration tablet, and wherein the formulation produces a pH of more than 4.9 after disintegration in 200 mL of neutral pure water.
46. The weakly effervescent disintegration formulation of form 18 comprising calcium carbonate, malic acid, cross linked povidone and ziprasidone, wherein the formulation is in the form of an oral disintegration tablet, and wherein the formulation produces a pH of more than 4.9 after disintegration in 200 mL of neutral pure water.
47. The weakly effervescent disintegration formulation of form 18 comprising calcium carbonate, an acid selected from the group consisting of malic acid, maleic acid, succinic acid, tartaric acid, ascorbic acid and citric acid, cross linked povidone and a triptan drug selected from sumatriptan or eletriptan, wherein the formulation is in the form of an oral disintegration tablet, and wherein the formulation produces a pH of more than 4.9 after disintegration in 200 mL of neutral pure water.
48. The weakly effervescent disintegration formulation of form 18 comprising calcium carbonate, malic acid, cross linked povidone and sumatriptan, wherein the formulation is in the form of an oral disintegration tablet, and wherein the formulation produces a pH of more than 4.9 after disintegration in 200 mL of neutral pure water.
49. The weakly effervescent disintegration formulation of form 18 comprising ibuprofen, calcium carbonate, an organic acid selected from the group consisting of malic acid, tartaric acid, succinic acid, maleic acid, ascorbic acid and citric acid, and cross linked povidone, wherein the formulation is in the form of an ODT, and wherein the formulation produces a pH of more than 4.9 after disintegration in 200 mL of neutral pure water.
50. The weakly effervescent disintegration formulation of form 18 comprising calcium carbonate, an organic acid selected from the group consisting of malic acid, maleic acid, succinic acid, tartaric acid, citric acid and ascorbic acid, and an anti-epileptic drug selected from the group consisting of phenobarbital, carbamazepine, topiramate, ethosuximide, gabapentin, and phenytoin, wherein the formulation is in the form of an oral disintegration tablet or a dispersible tablet, and wherein the formulation produces a pH of more than 4.9 after disintegration in 200 mL of neutral pure water.
51. The weakly effervescent disintegration formulation of form 18 comprising calcium carbonate, malic acid, phenobarbital, and cross linked povidone, wherein the formulation is in the form of an ODT, and wherein the formulation produces a pH of more than 4.9 after disintegration in 200 mL of neutral pure water.
52. The weakly effervescent disintegration formulation of form 18 comprising a base selected from the group consisting of sodium bicarbonate and potassium bicarbonate, phenytoin sodium, cross linked povidone, an acid selected from the group consisting of monosodium malate, monosodium maleate, monosodium succinate, monosodium citrate, monosodium tartrate, monopotassium malate, monopotassium maleate, monopotassium succinate, monopotassium citrate, and monopotassium tartrate, and cross linked povidone, wherein the formulation is in the form of an ODT, and wherein the formulation produces a pH of more than 4.9 after disintegration in 200 mL of neutral pure water.
53. The weakly effervescent disintegration formulation of form 18 comprising calcium carbonate, an acid selected from the group consisting of malic acid, maleic acid, succinic acid, tartaric acid, citric acid and ascorbic acid, topiramate, cross linked povidone, and magnesium stearate; wherein the formulation produces a suspension at a pH of more than 4.9 when disintegrated in 200 mL of neutral pure water.
54. The weakly effervescent disintegration formulation of form 18 comprising sildenafil, calcium carbonate, an acid selected from the group consisting of malic acid, tartaric acid, succinic acid, maleic acid, ascorbic acid and citric acid, and cross linked povidone, wherein the formulation is in the form of an ODT, and wherein the formulation produces a pH of more than 4.9 after disintegration in 200 mL of neutral pure water.
55. The weakly effervescent disintegration formulation of form 18 comprising calcium carbonate, an acid selected from the group consisting of malic acid, maleic acid, succinic acid, tartaric acid, citric acid and ascorbic acid, a 5-HT3 (5-hydroxytryptamine-3) receptor antagonist selected from the group consisting of ondansetron HCl, dolasetron HCl, granisetron HCl, palonosetron HCl, and ramosetron HCl, or a base thereof, and cross linked povidone, wherein the formulation is in the form of an ODT, and wherein the formulation produces a pH of more than 4.9 after disintegration in 200 mL of neutral pure water.
56. The weakly effervescent disintegration formulation of form 18 comprising ondansetron HCl, calcium carbonate, malic acid, and cross linked povidone, wherein the formulation is in the form of an oral disintegration tablet (ODT), and wherein the formulation produces a pH of more than 4.9 after disintegration in 200 mL of neutral pure water.
57. The weakly effervescent disintegration formulation of form 18 comprising granisetron HCl, calcium carbonate, malic acid, and cross linked povidone, wherein the formulation is in the form of an oral disintegration tablet (ODT), and wherein the formulation produces a pH of more than 4.9 after disintegration in 200 mL of neutral pure water.
58. A method of producing the pellet composition of celecoxib as defined in form 1, comprising the steps of:
   a) dissolving 1 part of celecoxib and 1 to 2 parts of HPMC E5 in 10 to 15 parts of a liquid alcohol selected ethanol or benzyl alcohol,
   b) placing 1 part to 2 parts of sugar pellets in a fluid bed;
   c) spraying the alcohol solution containing celecoxib and HPMC E5 of step a) into the fluid bed;
   d) mixing the resultant pellets of step c) with magnesium stearate; and
   e) filling the resultant pellets of step d) in a capsule or granules container, preferably a bag,
wherein the composition has a dissolution rate of more than 60% of celecoxib in 1000 mL of 0.25% sodium dodecyl sulfate/pH 7 sodium phosphate buffer solution when stirred under 50 RPM at 180 minutes, and wherein the composition is in the form of pellets.
59. A method of producing the composition of celecoxib as defined in form 1, comprising the steps of:
   a) dissolving 1 part of celecoxib and 1 to 6 parts of an organic acid in 10 to 20 parts of liquid alcohol, wherein the organic acid is selected from the group consisting of maleic acid, malic acid, tartaric acid, succinic acid, citric acid and ascorbic acid, and wherein the alcohol is selected from ethanol or benzyl alcohol;
   b) granulation: placing 1 part of cross linked povidone, 1 part of cross linked carboxymethyl sodium cellulose and a small amount of sodium dodecyl sulfate (about 20 mg per tablet) in a fluid bed;
   c) spraying the ethanol solution containing celecoxib and organic acid of step a) into the fluid bed;
   d) making the resultant powder of step c) into granules by spraying 8% aqueous PVPK30 into the fluid bed;
   e) drying the granules of step d) until the water content is below 4%;
   f) mixing the resultant granules of step e) together with magnesium stearate; and
   g) compressing the resultant granules of step f) into a tablet or filling the resultant granules of step f) into a capsule or granules container, preferably a bag,
wherein the composition has a dissolution rate of more than 60% of celecoxib in 1000 mL of 0.25% sodium dodecyl sulfate/pH 7 sodium phosphate buffer solution when stirred under 50 RPM at 120 minutes.
60. A method of treating hypocalcaemia or calcium deficiency in a subject comprising administering to the subject:
a therapeutically effective amount of the formulation of any one of forms 18 to 26, or
a weakly effervescent disintegration formulation comprising calcium carbonate, and an organic acid selected from the group consisting of malic acid, tartaric acid, succinic acid, maleic acid, ascorbic acid and citric acid; wherein the dosage of calcium carbonate per day is less than 80 mg, wherein the formulation of calcium carbonate produces a pH of more than 4.9 after disintegration in 200 mL of neutral pure water, wherein the formulation has a range of dissolution of calcium ions from 20% to 75% in 900 mL of neutral pure water, and wherein the formulation is in the form of a tablet, a capsule or granules.
61. A method of treating or preventing a disease or condition in a subject comprising administering to the subject the composition as defined in any one of forms 1 to 17 or the formulation of any one of forms 18 to 57, preferably the disease or condition is hypocalcaemia or calcium deficiency.
62. Use of the composition as defined in any one of forms 1 to 17 or the formulation of any one of forms 18 to 57 in the manufacture of a medicament for treating or preventing a disease or condition, preferably hypocalcaemia or calcium deficiency.
63. The composition as defined in any one of forms 1 to 17 or the formulation of any one of forms 18 to 57 for use in treating or preventing a disease or condition in a subject, preferably hypocalcaemia or calcium deficiency.
64. A weakly effervescent disintegration formulation comprising an active pharmaceutical ingredient, one or more excipients, and a weak acid-base pair,
wherein the formulation is in the form of granules, pellets, a capsule, a tablet, an oral disintegration tablet, or a dispersible tablet;
wherein the weak acid is an organic acid selected from a monosodium salt of a binary acid, a monopotassium salt of a binary acid, a monosodium salt of a ternary acid, a monopotassium salt of a ternary acid, ascorbic acid, a binary acid, or a mixture thereof;
the weak base is selected from calcium carbonate, sodium bicarbonate, potassium bicarbonate or lithium bicarbonate;
wherein the active pharmaceutical ingredient (API) is selected form the group consisting of a cyclooxygenase-2 (COX-2) inhibitor, calcium carbonate, glucosamine, acarbose, miglitol, a dihydropyridine calcium channel blocker, nimodipine, a triptan, an antihistamine, a sartan, a 5-HT3 antagonist antiemetic drug, an antifibrinolytic drugs, a non-classical antidepressant, a NSAID, a hormone contraceptive and an anti-epileptic, a salt thereof, or a mixture thereof; and
wherein the one or more excipients is selected from the group consisting of HPMC (hydroxypropyl methylcellulose), cross linked povidone, cross linked carboxymethyl sodium cellulose and magnesium stearate, or a mixture thereof.
65. The formulation of form 64, wherein the organic acid is selected from the group consisting of malic acid, maleic acid, succinic acid, tartaric acid, citric acid and ascorbic acid, a salt thereof, monosodium malate, monosodium maleate, monosodium succinate, monosodium citrate, monosodium tartrate, monopotassium malate, monopotassium maleate, monopotassium succinate, and monopotassium tartrate, or a mixture thereof.
66. The formulation of form 64 or 65, comprising cross linked povidone, cross linked carboxymethyl sodium cellulose and magnesium stearate, wherein the API is a cyclooxygenase-2 (COX-2) inhibitor selected from the group consisting of celecoxib and imrecoxib.
67. The formulation of form 64, wherein the COX-2 inhibitor is celecoxib.
68. The formulation of form 64, wherein the COX-2 inhibitor is imrecoxib.
69. The formulation of form 65, wherein the amount of organic acid is from 1 to 6 times the weight of celecoxib in the composition.
70. The formulation of form 67 or form 68, wherein the formulation has a dissolution rate of more than 60% of COX-2 inhibitor in 1000 mL of 0.25% sodium dodecyl sulfate/pH 7 sodium phosphate buffer solution under 50 RPM at 120 minutes after a dissolution test.
71. The formulation of form 67 or form 68, wherein the formulation is in the form of pellets or a capsule, the formulation further comprising sugar or mannitol, wherein the formulation comprises 1 part of COX-2 inhibitor, 1 to 2 parts of hydroxypropyl methylcellulose and 1 to 2 parts of pellets consisting of sugar or mannitol, wherein the formulation has a dissolution rate of COX-2 inhibitor of more than 60% in 1000 mL of neutral pure water containing 2.5 g of sodium dodecyl sulfate under 50 RPM at 180 minutes after a dissolution test.
72. The formulation of form 65, wherein the API is calcium carbonate.
73. The formulation of form 72, comprising cross linked povidone and magnesium stearate, wherein the organic acid is ascorbic acid, and the ratio of the weight of ascorbic acid to the weight of calcium carbonate is in the range of 1:1 to 3:1, wherein the formulation has a dissolution of calcium ions in the range of 20% to 65% in 900 mL of neutral water when stirred at a speed of 75 RPM when measured at 30 min.
74. The formulation of form 72, comprising cross linked povidone and magnesium stearate, wherein the organic acid is selected from the group consisting of tartaric acid, malic acid, maleic acid and succinic acid, and the molar ratio of the organic acid to calcium carbonate is in the range of 0.15:1.0 to 1.0:1.0, wherein when the formulation disintegrates in 200 mL of neutral pure water, a pH of greater than 4.9 is produced, and wherein the dissolution of calcium ions is in the range of 20% to 75% when stirred in 900 mL of neutral pure water at a speed of 75 RPM and measured at 30 min.
75. The formulation of form 72, comprising cross linked povidone and magnesium stearate, wherein the organic acid is citric acid, and the molar ratio of citric acid to calcium carbonate is less than 0.8:1, wherein when the formulation disintegrates in 200 mL of neutral water, a pH of greater than 4.9 is produced, and wherein the dissolution of calcium ions is in the range of 20% to 75% when stirred in 900 mL of neutral pure water at a speed of 75 RPM and measured at 30 min.
76. The formulation of form 72, comprising cross linked povidone and magnesium stearate, wherein the formulation further comprises glucosamine or a salt thereof, wherein the formulation has a dissolution of calcium ions of about 35% to 55% when stirred in 900 mL of neutral pure water at a speed of 75 RPM and measured at 30 min, wherein the pH of the suspension formed is from 6.5 to 7.5.
77. The formulation of form 72, comprising cross linked povidone, wherein the organic acid is selected from the group consisting of malic acid, maleic acid, tartaric acid, succinic acid, ascorbic acid and citric acid, further comprising an API selected from acarbose, miglitol, megestrol, or progesterone, and wherein the formulation produces a pH of more than 4.9 when disintegrated in 200 mL of neutral pure water, and wherein the formulation is in the form of an oral disintegration tablet.
78. The formulation of form 64, comprising sodium bicarbonate, an API selected from the group consisting of glucosamine hydrochloride and glucosamine sulfate, or a salt thereof, cross linked povidone, and magnesium stearate, wherein the formulation has a pH of the range of 6.5 to 7.5 when disintegrated in 200 mL of neutral pure water.
79. The formulation of form 64, comprising a weak base selected from the group consisting of sodium bicarbonate and potassium bicarbonate, an organic acid selected from the group consisting of monosodium malate, monosodium maleate, monosodium succinate, monosodium citrate, and monosodium tartrate, and cross linked povidone, and an API selected from nifedipine or nimodipine, wherein the formulation produces a pH of more than 4.9 when disintegrated in 200 mL of neutral pure water, and wherein the formulation is in the form of an oral disintegration tablet.
80. The formulation of form 64, comprising a weak base selected from the group consisting of sodium bicarbonate and potassium bicarbonate, an organic acid selected from the group consisting of monosodium malate, monosodium maleate, monosodium succinate, monosodium citrate, and monosodium tartrate, cross linked povidone, and cross linked carboxymethyl sodium cellulose, wherein the API is a sartan selected from the group consisting of losartan, candesartan, valsartan, telmisartan, fimasartan, and irbesartan, or a salt thereof, wherein the formulation produces a pH of more than 4.9 when disintegrated in 200 mL of neutral pure water, and wherein the formulation is in the form of an oral disintegration tablet or a dispersible tablet.
81. The formulation of form 64, wherein the weak base is a bicarbonate, comprising an organic acid selected from the group consisting of monosodium malate, monosodium maleate, monosodium succinate, monosodium citrate, monosodium tartrate, monopotassium malate, monopotassium maleate, monopotassium succinate, and monopotassium tartrate, and an excipient selected from the group consisting of cross linked povidone and cross linked carboxymethyl sodium cellulose, wherein the API is an antifibrinolytic drug selected from 6-aminocaproic acid or tranexamic acid, wherein the formulation produces a pH of more than 4.9 when disintegrated in 200 mL of neutral pure water, and wherein the formulation is in the form of a dispersible tablet.
82. The formulation of form 64, comprising calcium carbonate, an organic acid selected from the group consisting of malic acid, maleic acid, succinic acid, tartaric acid, citric acid and ascorbic acid, and an excipient selected from the group consisting of cross linked povidone and cross linked carboxymethyl sodium cellulose, wherein the API is a serotonin reuptake inhibitor selected from the group consisting of fluoxetine, paroxetine, sertraline, and fluvoxamine, or a salt thereof, wherein the formulation produces a pH of more than 4.9 when disintegrated in 200 mL of neutral pure water, and wherein the formulation is in the form of an oral disintegration tablet.
83. The formulation of form 64, comprising calcium carbonate, an organic acid selected from the group consisting of malic acid, maleic acid, succinic acid, tartaric acid, citric acid and ascorbic acid, cross linked povidone, and fexofenadine, wherein the formulation produces a pH of more than 4.9 when disintegrated in 200 mL of neutral pure water, and wherein the formulation is in the form of an oral disintegration tablet.
84. The formulation of form 64, comprising calcium carbonate, an organic acid selected from the group consisting of malic acid, maleic acid, succinic acid, tartaric acid, citric acid and ascorbic acid, and cross linked povidone, wherein the API is a non-classical antidepressant selected from quetiapine or ziprasidone, wherein the formulation produces a pH of more than 4.9 when disintegrated in 200 mL of neutral pure water, and wherein the formulation is in the form of an oral disintegration tablet.
85. The formulation of form 64, comprising calcium carbonate, an organic acid selected from the group consisting of malic acid, maleic acid, succinic acid, tartaric acid, citric acid and ascorbic acid, and cross linked povidone, wherein the API is a triptan drug selected from sumatriptan or eletriptan, wherein the formulation produces a pH of more than 4.9 when disintegrated in 200 mL of neutral pure water, and wherein the formulation is in the form of an oral disintegration tablet.
86. The formulation of form 64, comprising calcium carbonate, an organic acid selected from the group consisting of malic acid, maleic acid, succinic acid, tartaric acid, citric acid and ascorbic acid, cross linked povidone, and ibuprofen, wherein the formulation produces a pH of more than 4.9 when disintegrated in 200 mL of neutral pure water, and wherein the formulation is in the form of an oral disintegration tablet.
87. The formulation of form 64, comprising calcium carbonate, an organic acid selected from the group consisting of malic acid, maleic acid, succinic acid, tartaric acid, citric acid and ascorbic acid, and an excipient selected from the group consisting of cross linked povidone and magnesium stearate, or a mixture thereof, wherein the API is an anti-epileptic drug selected from the group consisting of phenobarbital, carbamazepine, topiramate, ethosuximide, gabapentin, and phenytoin, wherein the formulation produces a pH of more than 4.9 when disintegrated in 200 mL of neutral pure water, and wherein the formulation is in the form of an oral disintegration tablet.
88. The formulation of form 64, comprising calcium carbonate, an organic acid selected from the group consisting of malic acid, maleic acid, succinic acid, tartaric acid, citric acid and ascorbic acid, cross linked povidone, and sildenafil, wherein the formulation produces a pH of more than 4.9 when disintegrated in 200 mL of neutral pure water, and wherein the formulation is in the form of an oral disintegration tablet.
89. The formulation of form 64, comprising calcium carbonate, an organic acid selected from the group consisting of malic acid, maleic acid, succinic acid, tartaric acid, citric acid and ascorbic acid, and cross linked povidone, wherein the API is a 5-HT3 (5-hydroxytryptamine-3) receptor antagonist selected from the group consisting of ondansetron HCl, dolasetron HCl, granisetron HCl, palonosetron HCl, and ramosetron HCl, or a base thereof, wherein the formulation produces a pH of more than 4.9 when disintegrated in 200 mL of neutral pure water, and wherein the formulation is in the form of an oral disintegration tablet.
90. A method of producing a pellet composition comprising celecoxib, comprising the steps of:
   a) dissolving 1 part of celecoxib and 1 to 2 parts of an HPMC E5 in 10 to 15 parts of a liquid alcohol selected ethanol or benzyl alcohol, and
   b) placing 1 part to 2 parts of sugar pellet in a fluid bed;
   c) spraying the ethanol solution containing celecoxib and HPMC E5 of step a) into the fluid bed;
   d) mixing the resultant pellets of step c) with magnesium stearate; and
   e) filling the resultant pellet of step d) in a capsule or granules bag,
wherein the composition has a dissolution rate of more than 60% of celecoxib in 1000 mL of 0.25% sodium dodecyl sulfate/pH 7 sodium phosphate buffer solution when stirred under 50 RPM at 180 minutes.
91. A method of producing a pellet composition comprising celecoxib, comprising the steps of:
   a) 1 part of celecoxib and 1 to 6 parts of an organic acid is dissolved in 10 to 20 parts of liquid alcohol, wherein the organic acid is selected from the group consisting of maleic acid, malic acid, tartaric acid, succinic acid, citric acid and ascorbic acid, and wherein the alcohol is selected from ethanol or benzyl alcohol;
   b) granulation: 1 part of cross linked povidone, 1 part of cross linked carboxymethyl sodium cellulose and a small amount of sodium dodecyl sulfate (about 20 mg per tablet) are placed in the fluid bed;
   c) the ethanol solution containing celecoxib and organic acid of step a) is sprayed into the fluid bed;
   c) the resultant powder of step c) is made into granules by spraying 8% aqueous PVPK30 into the fluid bed;
   d) the granules are dried until the water content is below 4%;
   e) the resultant granules of step d) is mixed together with magnesium stearate; and
   f) the resultant granules of step e) are compressed into a tablet or are filled into a capsule or granules bag,
wherein the composition has a dissolution rate of more than 60% of celecoxib in 1000 mL of 0.25% sodium dodecyl sulfate/pH 7 sodium phosphate buffer solution when stirred under 50 RPM at 180 minutes.
92. A method of treating or preventing a disease or condition in a subject comprising administering to the subject the weakly effervescent disintegration formulation any one of forms 64 to 89.
93. Use of the weakly effervescent disintegration formulation any one of forms 64 to 89 in the manufacture of a medicament for treating or preventing a disease or condition.
94. The weakly effervescent disintegration formulation any one of forms 64 to 89 for use in treating or preventing a disease or condition in a subject.

1. A method of production of the composition of cyclooxygenase-2 (COX-2) inhibitors selected from the group consisting of celecoxib and imrecoxib, comprises a water soluble excipient selected from a group consisting of a HPMC (hydroxypropyl methylcellulose) and an organic acid which is selected from the group consisting of malic acid, maleic acid, succinic acid, tartaric acid, citric acid and ascorbic acid, wherein the COX-2 inhibitor and the water soluble excipient is dissolved in alcohol and mixed with expander and filler, such that the composition is in the form of a pellet, a capsule, granules or a tablet.

2. The method of production of the composition of cyclooxygenase-2 (COX-2) inhibitors of claim 1 comprises celecoxib, an organic acid selected from the group consisting of tartaric acid, maleic acid, succinic acid, malic acid, citric acid and ascorbic acid; the composition further comprises cross linked povidone, cross linked carboxymethyl sodium cellulose, and magnesium stearate, such that the composition is in the form of granules, a capsule or a tablet.

3. The method of production of the composition of cyclooxygenase-2 (COX-2) inhibitors of claim 1 comprises celecoxib, an organic acid selected from the group consisting of tartaric acid, maleic acid, succinic acid, malic acid, citric acid and ascorbic acid; the composition further comprises cross linked povidone, cross linked carboxymethyl sodium cellulose, such that the composition is in the form of granules, a capsule or a tablet which has a dissolution rate of more than 60% of celecoxib in 1000 mL of 0.25% sodium dodecyl sulfate/pH 7 sodium phosphate buffer solution under 50 RPM at 120 minutes after the dissolution test.

4. The method of production of the composition of cyclooxygenase-2 (COX-2) inhibitors of claim 1 comprises celecoxib, an organic acid selected from the group consisting of tartaric acid, maleic acid, succinic acid, malic acid, citric acid and ascorbic acid; wherein the amount of organic acid is from 1 to 6 times the weight of celecoxib in the composition; furthermore, the composition contains cross linked povidone, cross linked carboxymethyl sodium cellulose and the composition is in the form of granules, a tablet or a capsule which has a dissolution rate of more than 60% of celecoxib in 1000 mL of 0.25% sodium dodecyl sulfate/pH 7 sodium phosphate buffer solution under 50 RPM at 120 minutes after the dissolution test.

5. The method of production of the composition of cyclooxygenase-2 (COX-2) inhibitors of claim 1 comprises celecoxib, malic acid; the composition further comprises cross linked povidone, cross linked carboxymethyl sodium cellulose and magnesium stearate such that the composition is in the form of granules, a capsule or a tablet.

6. The method of production of the composition of cyclooxygenase-2 (COX-2) inhibitors of claim 1 comprises celecoxib, tartaric acid; the composition further comprises cross linked povidone, cross linked carboxymethyl sodium cellulose and magnesium stearate such that the composition is in the form of granules, a capsule or a tablet.

7. The method of production of the composition of cyclooxygenase-2 (COX-2) inhibitors of claim 1 comprises celecoxib, maleic acid; the composition further comprises cross linked povidone, cross linked carboxymethyl sodium cellulose and magnesium stearate such that the composition is in the form of a granule, a capsule or a tablet.

8. The method of production of the composition of cyclooxygenase-2 (COX-2) inhibitors of claim 1 comprises celecoxib, succinic acid; the composition further comprises cross linked povidone, cross linked carboxymethyl sodium cellulose and magnesium stearate such that the composition is in the form of granules, a capsule or a tablet.

9. The method of production of the composition of cyclooxygenase-2 (COX-2) inhibitors of claim 1 comprises celecoxib, citric acid; the composition further comprises cross linked povidone, cross linked carboxymethyl sodium cellulose and magnesium stearate, such that the composition is in the form of granules, a capsule or a tablet.

10. The method of production of the composition of cyclooxygenase-2 (COX-2) inhibitors of claim 1 comprises celecoxib, ascorbic acid; the composition further comprises cross linked povidone, cross linked carboxymethyl sodium cellulose and magnesium stearate, such that the composition is in the form of granules, a capsule or a tablet.

11. The method of production of the composition of cyclooxygenase-2 (COX-2) inhibitors of claim 1 comprises imrecoxib, an organic acid selected from the group consisting of maleic acid, succinic acid, tartaric acid, malic acid, citric acid and ascorbic acid or a mixture thereof; the composition further comprises cross linked carboxymethyl sodium cellulose, cross linked povidone and magnesium stearate, such that the composition is in the form of granules, a capsule or a tablet and has a dissolution of imrecoxib of more than 60% in 1000 mL of 0.25% sodium dodecyl sulfate/pH 7 sodium phosphate buffer solution under 50 RPM at 120 minutes after dissolution test.

12. The method of production of the composition of cyclooxygenase-2 (COX-2) inhibitors of claim 1 comprises celecoxib, an organic acid selected from the group consisting of maleic acid, succinic acid, tartaric acid, malic acid, citric acid and ascorbic acid; the composition further comprises cross linked povidone, cross linked carboxymethyl sodium cellulose, calcium carbonate, magnesium stearate, such that the composition is in the form of granules, a tablet, or a capsule and has a dissolution rate of celecoxib of more than 60% in 1000 mL of 0.25% sodium dodecyl sulfate/pH 7 sodium phosphate buffer solution under 50 RPM at 120 minutes after dissolution test.

13. The method of production of the composition of cyclooxygenase-2 (COX-2) inhibitors of claim 1 comprises imrecoxib, calcium carbonate, an organic acid selected from the group consisting of maleic acid, succinic acid, tartaric acid, malic acid, citric acid and ascorbic acid; the composition further comprises cross linked povidone, cross linked carboxymethyl sodium cellulose and magnesium stearate such that the composition is in the form of granules, a tablet or a capsule.

14. The method of production of the composition of cyclooxygenase-2 (COX-2) inhibitors of claim 1 comprises celecoxib, hydroxypropyl methylcellulose and pellet consisting of sugar or mannitol such that the composition is in the form of a pellet or a capsule.

15. The method of production of the composition of cyclooxygenase-2 (COX-2) inhibitors of claim 1 comprises 1 part of celecoxib, 1 to 2 parts of hydroxypropyl methylcellulose and 1 to 2 parts of pellet consisting of sugar or mannitol such that the composition is in the form of a pellet or a capsule and has a dissolution rate of celecoxib of more than 60% in 1000 mL of neutral pure water containing 2.5 g of sodium dodecyl sulfate under 50 RPM at 180 minutes after dissolution test.

16. The method of production of the composition of cyclooxygenase-2 (COX-2) inhibitors of claim 1 comprises imrecoxib, hydroxypropyl methylcellulose and pellet consisting of sugar or mannitol such that the composition is in the form of a pellet or a capsule.

17. The method of production of the composition of cyclooxygenase-2 (COX-2) inhibitors of claim 1 comprises 1 part of imrecoxib, 1 to 2 parts of hydroxypropyl methylcellulose and 1 to 2 parts of pellet consisting of sugar or mannitol such that the composition is in the form of a pellet or a capsule and has a dissolution rate of imrecoxib of more than 60% in 1000 mL of neutral pure water containing 2.5 g of sodium dodecyl sulfate under 50 RPM at 180 minutes after dissolution test.

18. A weakly effervescent disintegration formulation comprises a weak acid-base pair, wherein the weak acid is selected from monosodium salt of a binary acid, monopotassium salt of a binary acid, monosodium salt of a ternary acid, ascorbic acid, a binary acid, and mixtures thereof; and the weak base is selected from calcium carbonate, sodium bicarbonate, potassium bicarbonate and lithium bicarbonate and an API which is selected from the group consisting of calcium carbonate, glucosamine, acarbose, miglitol, dihydropyridine calcium channel blocker, nimodipine, triptans, antihistamines, sartans, 5-HT3 antagonist antiemetic drugs, antifibrinolytic drugs, non-classical antidepressants, NSAIDs, hormone contraceptives and anti-epileptics; wherein the formulation disintegrates in neutral pure water to produce a pH of greater than 4.9.

19. The weakly effervescent disintegration formulation of claim 18 comprises calcium carbonate, ascorbic acid, cross linked povidone, magnesium stearate, wherein the ratio of the weight of ascorbic acid to the weight of calcium carbonate is in the range of 1:1 to 3:1 and the composition is in the form of a capsule, a tablet, an oral disintegration tablet or a dispersible tablet disintegrating in 200 mL of neutral pure water to produce a pH of greater than 4.9 and has the dissolution of calcium ions in the range of 20% to 65% in 900 mL of neutral water under stirring at a speed of 75 RPM when measured at 30 min.

20. The weakly effervescent disintegration formulation of claim 18 comprises calcium carbonate, an organic acid selected from the group consisting of tartaric acid, malic acid, maleic acid and succinic acid and the ratio of these acid to calcium carbonate is in the range of 0.15:1.0 to 1.0:1.0 molar ratio and the composition is in the form of granules, a capsule, a tablet or a dispersible tablet and disintegrates in 200 mL of neutral pure water to produce a pH of greater than 4.9 and the dissolution of calcium ions is in the range of 20% to 75% in 900 mL of neutral pure water under stirring at a speed of 75 RPM when measured at 30 min.

21. The weakly effervescent disintegration formulation of claim 18 comprises calcium carbonate, a citric acid, cross linked povidone, a magnesium stearate, wherein the molar ratio of citric acid to calcium carbonate is less than 0.8:1 and the formulation is in the form of granules, a capsule, a tablet or a dispersible tablet and disintegrates in 200 mL of neutral water to produce a pH of greater than 4.9 and the formulation has a dissolution of calcium ions in the range of 20% to 75% in 900 mL of neutral pure water under stirring at a speed of 75 RPM when measured at 30 min.

22. The weakly effervescent disintegration formulation of claim 18 comprises calcium carbonate, maleic acid, cross linked povidone, and magnesium stearate, wherein the molar ratio of maleic acid to calcium carbonate is from 0.15:1 to 1:1 and the formulation is in the form of granules, a capsule, a tablet, oral disintegration tablet or a dispersible tablet disintegrating in 200 mL of neutral pure water to produce a pH of greater than 4.9 and has the dissolution of calcium ions in the range of 20% to 75% in 900 mL of neutral pure water under stirring at a speed of 75 RPM when measured at 30 min.

23. The weakly effervescent disintegration formulation of claim 18 comprises calcium carbonate, malic acid, cross linked povidone and magnesium stearate, wherein the molar ratio of malic acid to calcium carbonate is from 0.15:1 to 1:1 and the formulation is in the form of granules, a capsule, a tablet, an oral disintegration tablet or a dispersible tablet disintegrating in 200 mL of neutral pure water to produce a pH of greater than 4.9 and has the dissolution of calcium ions in the range of 20% to 75% in 900 mL of neutral pure water under stirring at a speed of 75 RPM when measured at 30 min.

24. The weakly effervescent disintegration formulation of claim 18 comprises calcium carbonate, tartaric acid, cross linked povidone and magnesium stearate, wherein the molar ratio of tartaric acid to calcium carbonate is from 0.15:1 to 1:1 and the formulation is in the form of granules, a capsule, a tablet, a dispersible tablet or an oral disintegration tablet disintegrating in 200 mL of neutral pure water to produce a pH of greater than 4.9 and has the dissolution of calcium ions in the range of 20% to 75% in 900 mL of neutral pure water under stirring at a speed of 75 RPM when measured at 30 min.

25. The weakly effervescent disintegration formulation of claim 18 comprises calcium carbonate, succinic acid, cross linked povidone and magnesium stearate, wherein the molar ratio of succinic acid to calcium carbonate is from 0.15:1 to 1:1 and the formulation disintegrates in 200 mL of neutral pure water to produce a pH of greater than 4.9 and has the dissolution of calcium ions in the range of 20% to 75% in 900 mL of neutral pure water under stirring at a speed of 75 RPM when measured at 30 min.

26. The weakly effervescent disintegration formulation of claim 18 comprises calcium carbonate, glucosamine hydrochloride or its salt, cross linked povidone, and magnesium stearate, wherein the formulation is in the form of granules or a tablet, which has a dissolution of calcium ions of about 35% to 55% in 900 mL of neutral pure water under stirring at a speed of 75 RPM when measured at 30 min; wherein the pH of the suspension formed is from 6.5 to 7.5.

27. The weakly effervescent disintegration formulation of claim 18 comprises sodium bicarbonate, glucosamine hydrochloride or its salt, cross linked povidone, magnesium stearate, wherein the composition is in the form of granules, a capsule, or a dispersible tablet, which has a pH of the range of 6.5 to 7.5 after disintegration in 200 mL of neutral pure water.

28. The weakly effervescent disintegration formulation of claim 18 comprises sodium bicarbonate, glucosamines sulfate, cross linked povidone, and magnesium stearate, wherein the formulation is in the form of granules, a capsule, or a dispersible tablet, which has a pH of the range of 6.5 to 7.5 in 200 mL of neutral pure water.

29. The weakly effervescent disintegration formulation of claim 18 comprises acarbose, calcium carbonate, an acid selected from the group consisting of malic acid, maleic acid, tartaric acid, succinic acid, ascorbic acid and citric acid, and cross linked povidone, wherein the composition is in the form of an oral disintegration tablet and produces a pH of more than 4.9 after disintegration in 200 mL of neutral pure water.

30. The weakly effervescent disintegration formulation of claim 18 comprises miglitol, calcium carbonate, an acid selected from the group consisting of malic acid, maleic acid, tartaric acid, succinic acid, ascorbic acid and citric acid, cross linked povidone, wherein the composition is in the form of an oral disintegration tablet and produces a pH of more than 4.9 after disintegration in 200 mL of neutral pure water.

31. The weakly effervescent disintegration formulation of claim 18 comprises megestrol, calcium carbonate, an acid selected from the group consisting of malic acid, maleic acid, tartaric acid, succinic acid, ascorbic acid and citric acid, and cross linked povidone, wherein the composition is in the form of an oral disintegration tablet and produces a pH of more than 4.9 after disintegration in 200 mL of neutral pure water.

32. The weakly effervescent disintegration formulation of claim 18 comprises progesterone, calcium carbonate, an acid selected from the group consisting of malic acid, maleic acid, tartaric acid, succinic acid, ascorbic acid and citric acid, and cross linked povidone, wherein the composition is in the form of an oral disintegration tablet and produces a pH of more than 4.9 after disintegration in 200 mL of neutral pure water.

33. The weakly effervescent disintegration formulation of claim 18 comprises a weak base selected from a group consisting of sodium bicarbonate and potassium bicarbonate, an acid selected from the group consisting of monosodium malate, monosodium maleate, monosodium succinate, monosodium citrate, monosodium tartrate, cross linked povidone and nifedipine, wherein the composition is in the form of an oral disintegration tablet which produces a pH of more than 4.9 after disintegration in 200 mL of neutral pure water.

34. The weakly effervescent disintegration formulation of claim 18 comprises a weak base selected from a group consisting of sodium bicarbonate and potassium bicarbonate, an acid selected from the group consisting of monosodium malate, monosodium maleate, monosodium succinate, monosodium citrate, monosodium tartrate, cross linked povidone and nimodipine, wherein the composition is in the form of an oral disintegration tablet which produces a pH of more than 4.9 after disintegration in 200 mL of neutral pure water.

35. The weakly effervescent disintegration formulation of claim 18 comprises a weak base selected from a group consisting of sodium bicarbonate and potassium bicarbonate, an acid selected from a group consisting of monosodium malate, monosodium maleate, monosodium succinate, monosodium citrate, monosodium tartrate, cross linked povidone, cross linked carboxymethyl sodium cellulose, and a sartan API selected from losartan, candesartan, valsartan, telmisartan, fimasartan, irbesartan and salts thereof; wherein the composition is in the form of an oral disintegration tablet or a dispersible tablet which produces a pH of more than 4.9 after disintegration in 200 mL of neutral pure water.

36. The weakly effervescent disintegration formulation of claim 18 comprises sodium bicarbonate, monosodium tartrate, cross linked carboxymethyl sodium cellulose and valsartan, wherein the composition is in the form of an oral disintegration tablet and produces a pH of more than 4.9 after disintegration in 200 mL of neutral pure water.

37. The weakly effervescent disintegration formulation of claim 18 comprises sodium bicarbonate, monosodium tartrate, cross linked carboxymethyl sodium cellulose and irbesartan, wherein the composition is in the form of an oral disintegration tablet and produces a pH of more than 4.9 after disintegration in 200 mL of neutral pure water.

38. The weakly effervescent disintegration formulation of claim 18 comprises a bicarbonate, an acid selected from a group consisting of monosodium malate, monosodium maleate, monosodium succinate, monosodium citrate, monosodium tartrate, monopotassium malate, monopotassium maleate, monopotassium succinate, monopotassium tartrate, cross linked povidone and an antifibrinolytic drug selected from 6-aminocaproic acid and tranexamic acid, wherein the composition produces a pH of more than 4.9 after disintegration in 200 mL of neutral pure water.

39. The weakly effervescent disintegration formulation of claim 18 comprises sodium bicarbonate, monosodium tartrate, cross linked carboxymethyl sodium cellulose and tranexamic acid, wherein the composition is in the form of a dispersible tablet and produces a pH of more than 4.9 after disintegration in 200 mL of neutral pure water.

40. The weakly effervescent disintegration formulation of claim 18 comprises calcium carbonate, an organic acid selected from the group consisting of malic acid, maleic acid, succinic acid, tartaric acid, citric acid and ascorbic acid, cross linked povidone, and a serotonin reuptake inhibitor selected from fluoxetine, paroxetine, sertraline, fluvoxamine and its salts thereof; wherein the composition is in the form of an oral disintegration tablet and produces a pH of more than 4.9 after disintegration in 200 mL of neutral pure water.

41. The weakly effervescent disintegration formulation of claim 18 comprises calcium carbonate, malic acid, cross linked carboxymethyl sodium cellulose, sertraline, wherein the composition is in the form of an oral disintegration tablet and produces a pH of more than 4.9 after disintegration in 200 mL of neutral pure water.

42. The weakly effervescent disintegration formulation of claim 18 comprises calcium carbonate, malic acid, cross linked carboxymethyl sodium cellulose, and fluvoxamine, wherein the composition is an oral disintegration tablet and produces a pH of more than 4.9 after disintegration in 200 mL of neutral pure water.

43. The weakly effervescent disintegration formulation of claim 18 comprises calcium carbonate, an organic acid selected from the group consisting of maleic acid, malic acid, succinic acid, tartaric acid, ascorbic acid and citric acid, cross linked povidone, and fexofenadine, wherein the composition is in the form of an oral disintegration tablet which produces a pH of more than 4.9 after disintegration in 200 mL of neutral pure water.

44. The weakly effervescent disintegration formulation of claim 18 comprises calcium carbonate, an organic acid selected from the group consisting of malic acid, maleic acid, succinic acid, tartaric acid, ascorbic acid and citric acid, cross linked povidone, and a non-classical antidepressant selected from quetiapine, ziprasidone, wherein the composition is in the form of an oral disintegration tablet which produces a pH of more than 4.9 after disintegration in 200 mL of neutral pure water.

45. The weakly effervescent disintegration formulation of claim 18 comprises calcium carbonate, malic acid, cross linked povidone and quetiapine, wherein the composition is in the form of an oral disintegration tablet and produces a pH of more than 4.9 after disintegration in 200 mL of neutral pure water.

46. The weakly effervescent disintegration formulation of claim 18 comprises calcium carbonate, malic acid, cross linked povidone and ziprasidone, wherein the composition is in the form of an oral disintegration tablet and produces a pH of more than 4.9 after disintegration in 200 mL of neutral pure water.

47. The weakly effervescent disintegration formulation of claim 18 comprises calcium carbonate, an acid selected from the group consisting of malic acid, maleic acid, succinic acid, tartaric acid, ascorbic acid and citric acid, cross linked povidone and a triptan drug selected from sumatriptan and eletriptan, wherein the formulation is in the form of an oral disintegration tablet and produces a pH of more than 4.9 after disintegration in 200 mL of neutral pure water.

48. The weakly effervescent disintegration formulation of claim 18 comprises calcium carbonate, malic acid, cross linked povidone and sumatriptan, wherein the composition is in the form of an oral disintegration tablet and produces a pH of more than 4.9 after disintegration in 200 mL of neutral pure water.

49. The weakly effervescent disintegration formulation of claim 18 comprises ibuprofen, calcium carbonate, an organic acid selected from the group consisting of malic acid, tartaric acid, succinic acid, maleic acid, ascorbic acid and citric acid, and cross linked povidone, such that the composition is in the form of an ODT and produces a pH of more than 4.9 after disintegrating in 200 mL of neutral pure water.

50. The weakly effervescent disintegration formulation of claim 18 comprises calcium carbonate, an organic acid selected from the group consisting of malic acid, maleic acid, succinic acid, tartaric acid, citric acid and ascorbic acid, and an anti-epileptic drug selected from the group consisting of phenobarbital, carbamazepine, topiramate, ethosuximide, gabapentin, and phenytoin, such that the composition is in the form of an oral disintegration tablet or a dispersible tablet and produces a pH of more than 4.9 after disintegrating in 200 mL of neutral pure water.

51. The weakly effervescent disintegration formulation of claim 18 comprises calcium carbonate, malic acid, phenobarbital, cross linked povidone, such that the composition is in the form of an ODT and produces a pH of more than 4.9 after disintegrating in 200 mL of neutral pure water.

52. The weakly effervescent disintegration formulation of claim 18 comprises a base selected from a group consisting of sodium bicarbonate and potassium bicarbonate, phenytoin sodium, cross linked povidone, an acid selected from the group consisting of monosodium malate, monosodium maleate, monosodium succinate, monosodium citrate, monosodium tartrate, monopotassium malate, monopotassium maleate, monopotassium succinate, monopotassium citrate, monopotassium tartrate, and cross linked povidone, such that the composition is in the form of an ODT and produces a pH of more than 4.9 after disintegrating in 200 mL of neutral pure water.

53. The weakly effervescent disintegration formulation of claim 18 comprises calcium carbonate, an acid selected from the group consisting of malic acid, maleic acid, succinic acid, tartaric acid, citric acid and ascorbic acid, topiramate, cross linked povidone, and magnesium stearate; wherein the formulation disintegrates in 200 mL of neutral pure water and the pH of the suspension formed is more than 4.9.

54. The weakly effervescent disintegration formulation of claim 18 comprises sildenafil, calcium carbonate, an acid selected from the group consisting of malic acid, tartaric acid, succinic acid, maleic acid, ascorbic acid and citric acid, cross linked povidone, such that the composition is in the form of an ODT and produces a pH of more than 4.9 after disintegrating in 200 mL of neutral pure water.

55. The weakly effervescent disintegration formulation of claim 18 comprises calcium carbonate, an acid selected from the group consisting of malic acid, maleic acid, succinic acid, tartaric acid, citric acid and ascorbic acid, a 5-HT3 (5-hydroxytryptamine-3) receptor antagonist selected from the group consisting of ondansetron HCl, dolasetron HCl, granisetron HCl, palonosetron HCl, ramosetron HCl or their base, and cross linked povidone, such that the formulation is in the form of an ODT and produces a pH of more than 4.9 after disintegrating in 200 mL of neutral pure water.

56. The weakly effervescent disintegration formulation of claim 18 comprises ondansetron HCl, calcium carbonate, malic acid, and cross linked povidone, such that the composition is in the form of an oral disintegration tablet (ODT) and produces a pH of more than 4.9 after disintegrating in 200 mL of neutral pure water.

57. The weakly effervescent disintegration formulation of claim 18 comprises granisetron HCl, calcium carbonate, malic acid, and cross linked povidone, such that the composition is in the form of an oral disintegration tablet (ODT) and produces a pH of more than 4.9 after disintegrating in 200 mL of neutral pure water.

58. A method of production of the pellet composition of cyclooxygenase-2 (COX-2) inhibitors of celecoxib comprises:
a) 1 part of celecoxib and 1 to 2 parts of an HPMC E5 is dissolved in 10 to 15 parts of liquid alcohol selected from a group consisting of ethanol and benzyl alcohol, and
b) 1 part to 2 parts of sugar pellet is put in the fluid bed; the ethanol solution containing celecoxib and HPMC E5 is sprayed into the fluid bed; the pellet made and magnesium stearate are mixed together and then is filled into a capsule or granules bag, which has a dissolution rate of more than 60% of celecoxib in 1000 mL of 0.25% sodium dodecyl sulfate/pH 7 sodium phosphate buffer solution under 50 RPM at 180 minutes after dissolution test
wherein the composition is in the form of pellets.

59. A method of production of the composition of cyclooxygenase-2 (COX-2) inhibitors of celecoxib comprises:
a) 1 part of celecoxib and 1 to 6 parts of an organic acid selected from the group consisting of maleic acid, malic acid, tartaric acid, succinic acid, citric acid and ascorbic acid is dissolved in 10 to 20 parts of liquid alcohol selected from a group consisting of ethanol and benzyl alcohol,
b) granulation: 1 part of cross linked povidone, 1 part of cross linked carboxymethyl sodium cellulose and a small amount of sodium dodecyl sulfate (about 20 mg per tablet) is put in the fluid bed; the ethanol solution containing celecoxib and organic acid is sprayed into the fluid bed,
c) the powder is made into granules with 8% aqueous PVPK30 in fluid bed and dried until the water content is below 4%,
d) the resultant granules and magnesium stearate are mixed together and is then compressed into a tablet or is filled into a capsule or granules bag, which has a dissolution rate of more than 60% of celecoxib in 1000 mL of 0.25% sodium dodecyl sulfate/pH 7 sodium phosphate buffer solution under 50 RPM at 120 minutes after dissolution test.

60. A method of treating hypocalcaemia or calcium deficiency in a subject by administration of a weakly effervescent disintegration formulation of calcium carbonate with an organic acid selected from the group consisting of malic acid, tartaric acid, succinic acid, maleic acid, ascorbic acid and citric; wherein the dosage of calcium carbonate per day is less than 80 mg, the formulation of calcium carbonate produces a pH of more than 4.9 after disintegrating in 200 mL of neutral pure water and has a range of dissolution of calcium ions from 20% to 75% in 900 mL of neutral pure water, such that the formulation is in the form of a tablet, a capsule or granules.
